(19) United States
(12) Patent Application Publication
(10) Pub. No.: US 2024/0025939 A1
(43) Pub. Date: Jan. 25, 2024

(54) HIGH SEQUENCE FIDELITY NUCLEIC ACID SYNTHESIS AND ASSEMBLY

(71) Applicants: LIFE TECHNOLOGIES CORPORATION, Carlsbad, CA (US); THERMO FISHER SCIENTIFIC GENEART GMBH, Regensburg (DE)

(72) Inventors: Robert POTTER, San Marcos, CA (US); Nikolai NETUSCHIL, Regensburg (DE)

(21) Appl. No.: 17/909,091

(22) PCT Filed: Mar. 5, 2021
(86) PCT No.: PCT/US2021/021104

§ 371 Date: Sep. 2, 2022

Related U.S. Application Data
(60) Provisional application No. 62/986,209, filed on Mar. 6, 2020.

(51) Int. Cl. C07H 21/00; C12Q 1/686; C12N 9/22

(52) U.S. Cl. CPC C07H 21/00; C12Q 1/686; C12N 9/22

(57) ABSTRACT

The present disclosure generally relates to compositions and methods for the synthesis of nucleic acid molecules with low error rates. Provided, as examples, are compositions and methods for high throughput synthesis and assembly of nucleic acid molecules, in many instances, with high sequence fidelity. In many instances, thermostable mismatch recognition proteins (e.g., thermostable mismatch binding protein, thermostable mismatch endonucleases) will be present in compositions and use methods provided.

### FIELD OF THE INVENTION

The present disclosure generally relates to compositions and methods for the synthesis of nucleic acid molecules with low error rates. Provided, as examples, are compositions and methods for high throughput synthesis and assembly of nucleic acid molecules, in many instances, with high sequence fidelity. In many instances, thermostable mismatch recognition proteins (e.g., thermostable mismatch binding protein, thermostable mismatch endonucleases) will be present in compositions and use methods provided.

### BACKGROUND

Over the years, gene synthesis has become more cost effective and efforts to develop high throughput synthesis platforms in which the nucleic acid molecules produced have high sequence fidelity.

Biological materials that can be used in processes for generating nucleic acid molecules produced have high sequence fidelity have evolved along with the organisms that produce these materials. Such biological materials include DNA polymerases with proof reading abilities and materials involved in various pathways for correction of nucleic acid sequence errors (e.g., mismatch endonucleases, mismatch binding proteins, etc.).

With progress in genetic engineering, a need for the generation of larger nucleic acid molecules has developed. In many instances, nucleic acid assembly methods start with the synthesis of relatively short nucleic acid molecules (e.g., chemically synthesized oligonucleotides), followed by the generation of double-stranded fragments or sub-assemblies (e.g., by annealing and elongating multiple overlapping oligonucleotides), and often proceeds to build larger assemblies such as genes, operons or even functional biological pathways (e.g., by ligation, enzymatic elongation, recombination or a combination thereof). The present disclosure generally relates to compositions and methods for the assembly of nucleic acid molecules having high sequence fidelity.

### SUMMARY

The present disclosure relates, in part, to compositions and methods for the assembly (e.g., by assembly PCR) and amplification of nucleic acid molecules having high nucleotide sequence fidelity. Compositions and methods set out herein may contain or employ proteins that can detect and/or eliminate nucleic acid molecules that contain errors (e.g., DNA polymerases, mismatch endonucleases, mismatch binding proteins, etc.).

In some aspects, provided herein are methods for generating error corrected populations of nucleic acid molecules. Such methods may include: (a) assembling oligonucleotides with regions of terminal sequence complementarity (single-stranded regions that, upon hybridization, form double-stranded regions of from about 10 to about 30, from about 12 to about 30, from about 15 to about 30, from about 20 to about 30, from about 15 to about 40, from about 6 to about 20, from about 8 to about 25, etc., base pairs in length) by primary assembly PCR to form a population of assembled nucleic acid molecules, and (b) amplifying the population of assembled nucleic acid molecules formed in step (a) by primary amplification to form a population of amplified assembled nucleic acid molecules. In some instances, the population of amplified assembled nucleic acid molecules may contain fewer than two errors per 1,000 base pairs (e.g., from about two to about 0.01, from about two to about 0.05, from about two to about 0.08, from about two to about 0.1, from about two to about 0.5, from about two to about 0.75, from about one to about 0.01, from about one to about 0.05, from about one to about 0.1, from about two to about 0.001, from about one to about 0.001, from about 0.5 to about 0.001, from about 0.1 to about 0.001, etc., errors per 1,000 base pairs). In some instances, steps (a) and/or (b) above may be performed in the presence of one or more (e.g., from one to ten, one to eight, one to five, one to three, one to two, etc.) thermostable mismatch recognition proteins. In some aspects, at least one of the one or more thermostable mismatch recognition proteins is a thermostable mismatch binding protein, such as, for example, a thermostable mismatch binding protein selected from a mismatch binding protein having an amino acid sequence set out in Table 13 or Table 15. In some aspects, at least one of the one or more thermostable mismatch recognition proteins is a thermostable mismatch endonuclease, such as a mismatch endonuclease selected from a mismatch endonuclease having an amino acid sequence set out in Table 12 or Table 15 (e.g., TkoEndoMS, PfuEndoMS, etc.).

In some instances, a high-fidelity DNA polymerase may be used in methods set out herein. Further in more specific instances, a high-fidelity DNA polymerase may be used in steps (a) and/or (b) set out in the above methods for generating error corrected populations of nucleic acid molecules. Further, the high-fidelity DNA polymerase may be a component of an error reducing polymerase reagent. Error reducing polymerase reagents may comprise one or more (e.g., from one to ten, one to eight, one to five, one to three, one to two, etc.) amine compounds, such as one or more amine compounds are selected from the group consisting of (a) dimethylamine hydrochloride, (b) diisopropylamine hydrochloride, (c) ethyl(methyl)amine hydrochloride, and (d) trimethylamine hydrochloride.

In specific variations of methods set out herein and in the above methods for generating error corrected populations of nucleic acid molecules, at least one of the one or more thermostable mismatch recognition proteins may be present in step (a). Further, in some instances, at least one of the one or more thermostable mismatch recognition proteins may be present in step (b). Additionally, one or more (e.g., from one to ten, one to eight, one to five, one to three, one to two, etc.) error correction step may be performed after primary amplification. Also, post-primary amplification of the population of amplified assembled nucleic acid molecules may performed after step (b). In some instances, the population of amplified assembled nucleic acid molecules may be contacted with one or more mismatch recognition proteins prior to the post-primary amplification. Additionally, the at least one of the one or more mismatch recognition proteins may a mismatch endonuclease, such as one or more (e.g., from one to ten, one to eight, one to five, one to three, one to two, etc.) non-thermostable mismatch endonuclease (e.g., T7 endonuclease I, CEL II nuclease, CEL I nuclease, and/or T4 endonuclease VII).

Methods set out herein are also directed to the generation of populations of amplified assembled nucleic acid molecules that comprise subfragments of larger nucleic acid molecules. Further, in some instances, such populations of amplified assembled nucleic acid molecules may be combined with one or more (e.g., from one to ten, one to eight, one to five, one to three, one to two, etc.) additional nucleic acid molecules that are also subfragments of larger nucleic acid molecule, to form nucleic acid molecule pools. In some instances, the nucleic acid molecules of such nucleic acid molecule pools may be assembled by secondary assembly PCR to form the larger nucleic acid molecules. In some instances, the subfragments may be contacted with the one or more mismatch recognition proteins prior to or during assembly by secondary assembly PCR. Further, the larger nucleic acid molecule may be heat denatured, then renatured, followed by contacting with the one or more (e.g., from one to ten, one to eight, one to five, one to three, one to two, etc.) mismatch recognition proteins. Additionally, at least one (e.g., from one to ten, one to eight, one to five, one to three, one to two, etc.) of the one or more mismatch recognition proteins may be a mismatch binding protein, such as a mismatch binding protein that is bound to a solid support. Thus, methods set out herein include methods for the separation of nucleic acid molecule which contain errors from those that do not contain errors. In some instances, the population of amplified assembled nucleic acid molecules may be sequenced. Such sequencing may be performed to determine whether errors are present and, if so, how many errors and what types(s) of errors there are.

Also provided herein are compositions, such as compositions that may be used in methods et out herein. In some instances, compositions set out herein may comprise one or more (e.g., from one to ten, one to eight, one to five, one to three, one to two, etc.) thermostable mismatch recognition protein, one or more (e.g., from one to ten, one to eight, one to five, one to three, one to two, etc.) DNA polymerase, and one or more (e.g., from one to ten, one to eight, one to five, one to three, one to two, etc.) amine compound. Further, at least one of the one or more amine compound may be selected from the group consisting of (a) dimethylamine hydrochloride, (b) diisopropylamine hydrochloride, (c) ethyl(methyl)amine hydrochloride, and/or (d) trimethylamine hydrochloride.

Compositions set out herein may further comprise two or more nucleic acid molecules (e.g., two or more nucleic acid molecules are subfragments of a larger nucleic acid molecule). Further, the two or more nucleic acid molecules may be single-stranded. Such single-stranded nucleic acid molecules may vary greatly in length but, in many instances, will be between less than 100 (e.g., from about 35 to about 90, from about 35 to about 80, from about 35 to about 70, from about 35 to about 65, from about 40 to about 90, from about 30 to about 60, from about 30 to about 65, etc.) nucleotides in length.

Compositions set out herein may further comprise two or more nucleic acid molecules wherein at least one of the two or more nucleic acid molecules is single-stranded and wherein at least one of the two or more nucleic acid molecules is double-stranded.

In some compositions set out herein, at least one of the thermostable mismatch recognition protein may be a thermostable mismatch endonuclease, such as a thermostable mismatch endonuclease having an amino acid sequence set out in Table 12 or Table 15 (e.g., TkoEndoMS, PfuEndoMS, etc.), as well as variants thereof having at least 80% (e.g., at least from about 80% to about 99%, from about 80% to about 95%, from about 80% to about 90%, from about 85% to about 95%, from about 90% to about 99%, from about 92% to about 99%, from about 95% to about 99%, from about 97% to about 99%, etc.) sequence identity thereto.

In some specific instances, compositions and methods provided herein may contain or use mismatch specific endonucleases that share at least 30%, 40%, 50%, or 60% (e.g., from about 30% to about 70%, from about 30% to about 60%, from about 30% to about 50%, from about 30% to about 45%, from about 30% to about 40%, etc.) amino acid sequence identity with TkoEndoMS (SEQ ID NO: 3). Examples of such mismatch specific endonucleases are PisEndoMS (SEQ ID: 11) or SacEndoMS (SEQ ID: 12).

In some compositions set out herein at least one of the thermostable mismatch recognition protein may be a thermostable mismatch binding protein, such as a thermostable mismatch binding protein having an amino acid sequence set out in Table 13 or Table 15, as well as variants thereof having at least 80% (e.g., at least from about 80% to about 99%, from about 80% to about 95%, from about 80% to about 90%, from about 85% to about 95%, from about 90% to about 99%, from about 92% to about 99%, from about 95% to about 99%, from about 97% to about 99%, etc.) sequence identity thereto.

Also set out herein are methods of generating nucleic acid molecules with a predetermined sequence. In some instances, such methods may comprise: (a) providing a plurality of single-stranded oligonucleotides with complementary overlapping regions, each of the single-stranded oligonucleotides comprising a sequence region of the target nucleic acid molecule, wherein the plurality of single-stranded oligonucleotides comprises: (i) a plurality of internal oligonucleotides having overlapping sequence regions with two other oligonucleotides in the plurality, and (ii) two terminal oligonucleotides designed to be positioned at the 5′ and 3′ terminal ends of the full-length nucleic acid molecule and having an overlapping sequence region with one of the internal oligonucleotides in the plurality, (b) assembling the plurality of oligonucleotides by primary assembly PCR to obtain assembled double-stranded nucleic acid assembly products, (c) combining at least a portion of the assembly products obtained in step (b) with a pair of primers. In some instances, the primers of the pair may be designed to bind to the 5′ and 3′ terminal ends of the assembly products and performing a PCR amplification reaction to produce amplified assembly products. Further, in some instances, step (b) and/or step (c) may be conducted in the presence of one or more thermostable mismatch recognition protein.

Further set out herein are methods of generating nucleic acid molecules with a predetermined sequence further comprising (d) conducting one or more error correction steps. In some instances, such error correction steps may comprise: (i) denaturing and reannealing the amplified assembly products of step (c) to generate one or more mismatch containing double-stranded nucleic acids, (ii) treating the mismatch containing double-stranded nucleic acids with one or more mismatch recognition protein, and (iii) optionally, conducting an amplification reaction. In some instances, the mismatch recognition protein(s) used in step (d) is a mismatch endonuclease (e.g., T7 endonuclease I) or a mismatch binding protein (e.g., MutS). Further, the thermostable mismatch endonuclease(s) employed may be derived from hyperthermophilic Archaea, optionally, wherein the hyperthermophilic archaeon is Pyrococcus furiosus or Pyrococcus abyssi. Additionally, the thermostable mismatch recognition protein(s) may be selected from the group of proteins having an amino acid sequence set out in Table 12, 13, or 15, and variants thereof having at least 80% (e.g., at least from about 80% to about 99%, from about 80% to about 95%, from about 80% to about 90%, from about 85% to about 95%, from about 90% to about 99%, from about 92% to about 99%, from about 95% to about 99%, from about 97% to about 99%, etc.) sequence identity thereto.

In some instances, one or more of the thermostable mismatch recognition protein employed may be produced and/or obtained by in vitro transcription/translation. In other instances, one or more of the thermostable mismatch recognition protein employed may be produced and/or obtained by cellular expression.

When polymerases are present in compositions and used in methods set out herein, these polymerases may be high fidelity DNA polymerases. Thus, provided herein are methods such as methods of generating nucleic acid molecules with a predetermined sequence set out above, wherein one or more of steps (b), (c) and (d) (iii) may be conducted in the presence of a high fidelity DNA polymerase, optionally, wherein the polymerase may be selected from the group consisting of PHUSION™ DNA polymerase (PHUSION™), PLATINUM™ SUPERFI™ II DNA polymerase (SUPERF^{I}™ II), Q5 DNA Polymerase, and P^{RIME}STAR GXL DNA Polymerase. Additionally, one or more of steps (b), (c) and (d) (iii) may be conducted in the presence of a high fidelity DNA polymerase, optionally, wherein the polymerase is a polymerase have an amino acid sequence selected from the group consisting of: (1) DNA Polymerase 1, (2) DNA Polymerase 2, (3) DNA Polymerase 3, (4) DNA Polymerase 4, (5) DNA Polymerase 5, (6) DNA Polymerase 6, (7) DNA Polymerase 7 set out in Table 14.

In some variations of, for example, the above methods of generating nucleic acid molecules with a predetermined sequence, two or more amplified assembly products may be pooled prior to conducting the one or more error correction steps. Additional variations may further comprise treating the amplified assembly products with an exonuclease prior to the one or more error correction steps, optionally, wherein the exonuclease is Exonuclease I.

### BRIEF DESCRIPTION OF THE DRAWINGS

A better understanding of the features and advantages of subject matter set out herein may be obtained by reference to the following detailed description that sets forth illustrative aspects, in which the principles of subject matter set out herein are utilized, and the accompanying drawings of which:

FIGS. 1A to 1B show a comparison of two nucleic acid assembly workflows. FIG. 1A is a schematic of a standard workflow for assembling a nucleic acid molecule from single-stranded overlapping oligonucleotides comprising the steps of: oligonucleotide synthesis, oligonucleotide assembly PCR, and assembly PCR of reaction mixture generate subfragments (collectively primary assembly PCR); amplification of the assembly product (primary amplification); purification of amplified product; nuclease treatment, as an example, to generate complementary overhangs (e.g., generated by Type IIs endonuclease mediated cleavage); and vector insertion and transformation. FIG. 1B is a schematic of one variation of a sequence elongation and ligation reaction according to methods set out herein. Such reaction will often be performed as a “one pot” reaction because the assembly PCR (primary assembly PCR), the amplification (primary amplification), and the vector insertion steps can be performed in a single sealed vessel (e.g., a single sealed tube). In the workflow of FIG. 1B, vector termini serve as amplification primers.

FIG. 2 is a schematic of a PCR-based process for assembling and amplifying nucleic acid molecules. (a) Overlapping forward and reverse oligonucleotides are extended in the first PCR cycle. (b) Extended assembly products anneal to each other and are further extended in the second cycle. (c) Further extensions take place in subsequent PCR cycles and assembly product accumulates. The assembly processes in this figure are referred to herein as “Primary Assembly PCR” (labeled “A”). Two terminal oligonucleotides (1) and (2) can also be universal primers. Further, the terminal oligonucleotides may be added to primary assembly PCR products or the primary assembly PCR products can be added to another tube where they are then mixed with the terminal primers. Further, vector ends can be used instead of terminal oligonucleotides (see FIG. 1B). The final amplification step in this figure using the two terminal oligonucleotides is referred to herein as “Primary Amplification” (labeled “B”).

FIG. 3 is a schematic of an exemplary workflow for synthesis of error corrected nucleic acid molecules.

FIG. 4 shows a workflow schematic in which oligonucleotides are amplified then error-corrected and assembled into longer nucleic acid molecules.

FIGS. 5A to 5B show workflow schematics involving double error correction and amplification-based assembly of nucleic acid molecules generated by PCR (e.g., previously assembled nucleic acid molecules). In one variation (FIG. 5A), error correction is performed using one or more endonucleases in two locations in the workflow. Nine line number labels are included in FIG. 5A for reference in the specification. In another variation (FIG. 5B), error correction in two different locations in the workflow is performed using one or more endonucleases in the first round and a mismatch binding protein in the second round. As in FIG. 5A, nine line number labels are included in FIG. 5B for reference in the specification.

FIG. 6 shows a schematic representation of a workflow employing bead bound mismatch binding proteins for the separation of nucleic acid molecules that contain mismatches from those that do not contain mismatches. NMM refers to a non-mismatched nucleic acid molecule and MM refers to a mismatched nucleic acid molecule.

FIG. 7 shows error rate data (total errors) generated using various conditions determined by experimentation. With respect to this figure, the term “Assembly” refers to primary assembly PCR (see, e.g., FIG. 2 upper portion labeled A). The term “Amplification” refers to primer based primary amplification of assembled nucleic acid molecules (see, e.g., FIG. 2 lower portion labeled B). The term “Error Correction” refers to whether a post-primary amplification T7 Endonuclease I (T7NI) mediated error correction step was performed, which, in this instance, is secondary amplification. The notation in the “Assembly” and “Amplification” columns denote whether a wild-type mismatch endonuclease from Thermococcus kodakarensis (referred to herein as “TkoEndoMS”) in Ishino et al., Nucl. Acids Res. 44:2977-2989 (2016)) was included during assembly PCR and/or amplification. The column labeled “Sequenced Fragments” refers to the number of sets of fragments having different sequences that were tested. The “Error Rate” shown is the average of the data. The term “Benchmark” refers to the error rate with identical oligonucleotides but no error correction, as determined in separate experiments. Also represented in the table is the numerical average of all eight Benchmark values. Note: Run Nos. 1 through 8 were each performed with sets of oligonucleotides that differed in nucleotide sequence to allow for single run, next generation sequencing.

FIG. 8 is a graphical representation showing total error data points used to generate the data in FIG. 7. The numerical and letter descriptions on the lower axis of FIG. 8 correlate with the two columns on the left of FIG. 7. Each data point represents the number of errors per base pair for each of the nucleic acid molecule populations analyzed. The box on each vertical line represents the region of the vertical line where half of the data points fall. The horizontal line in the box represents the median. This figure shows the total number of errors present in the individual nucleic acid molecules. Thus, each data point represents the average number of errors for nucleic acid molecule designed to have the same nucleotide sequence. By way of analysis, the further from the lower axis, the fewer the number of errors present.

FIG. 9 is a graphical representation similar to that of FIG. 8 but instead of total errors being represented, the numbers of deletions is represented.

FIG. 10 is a graphical representation similar to that of FIG. 8 but instead of total errors being represented, the numbers of insertions is represented.

FIG. 11 is a graphical representation similar to that of FIG. 8 but instead of total errors being represented, the numbers of substitutions is represented.

FIG. 12A to FIG. 12D show specific types of errors present in two samples. In one sample (FIGS. 12A and 12B) nucleic acid molecules were assembled and amplified with no error correction. In the other sample (FIGS. 12C and 12D) nucleic acid molecules were assembled and amplified with TkoEndoMS error correction. No T7NI error correction was performed on either sample. The types of mismatches set out in FIGS. 12B and 12D are as follows: TS1=G-T, C-A, TS2=A-C, G-T, TV1=C-T, G-A, TV2=A-A, T-T, TV3=G-G, C-C, and TV4=T-C, A-G. “TS” refers to transitions and “TV” refers to transversions. Overall error rates were as follows: FIG. 12A—1 in 349 bases (Standard Deviation (SD): 1 in 99 bases) and FIG. 12C—1 in 488 bases (SD: 1 in 210 bases). Overall substitutions rates were as follows: FIG. 12B—1 in 647.8 bases and FIG. 12D—1 in 242.5 bases.

FIG. 13 shows data generated where sample sets of nucleic acid molecules were assembled and amplified with no error correction and PHUSION™ DNA polymerase (Thermo Fisher Scientific, cat. no. F530S) (A-C) or where TkoEndoMS was used during assembly PCR and amplification and PLATINUM™ SUPERFI™ II DNA polymerase (Thermo Fisher Scientific, cat. no. 12361010) (D-F) was used for both assembly PCR and amplification. No T7NI error correction was performed on either sample.

FIGS. 14A to 14D show specific types of errors present in two samples. In one sample (FIGS. 14A and 14B) nucleic acid molecules were assembled (primary assembly PCR) and amplified (primary amplification with no error correction and using PHUSION™ DNA polymerase. In the other sample (FIGS. 14C and 14D) nucleic acid molecules were assembled and amplified with TkoEndoMS error correction and PLATINUM™ SUPERFI™ II DNA polymerase. No T7NI error correction was performed on either sample. Overall error rates were as follows: FIG. 14A—1 in 251 bases (Standard Deviation (SD): 1 in 25 bases) and FIG. 14C—1 in 670 bases (SD: 1 in 112 bases). Overall substitutions rates were as follows: FIG. 14B—1 in 462.4 bases and FIG. 14D—1 in 565.2 bases.

FIG. 15 shows the amino acid sequence of TkoEndoMS (SEQ ID NO: 1) with an N-terminal signal peptide and a C-terminal histidine purification tag and the nucleotide sequence of a codon optimized nucleic acid molecule encoding this protein (SEQ ID NO: 2).

FIG. 16 shows an amino acid sequence alignment of Thermococcus kodakarensis EndoMS (referred to herein as “TkoEndoMS”) (SEQ ID NO: 3) and Pyrococcus furiosus EndoMS referred to herein as “PfuEndoMS”) (SEQ ID NO: 4). The amino acid sequences of these two proteins share 69% sequence identity.

FIG. 17A shows data derived from thirty nucleic acid molecules assembled using PHUSION™ (“before”) or PLATINUM™ SUPERFI™ II (“after”) DNA polymerase. This figure shows relative change in error rate for individual fragments after vs. before. The actual error rates and standard deviations for the individual fragments are 1 in 339±52 base pairs (bps) for before and 1 in 447±89 bps after, or an average improvement in error rate of 32.3±20.1%. PLATINUM™ SUPERFI™ II DNA polymerase is shown to result in lower error rates compared to PHUSION™ DNA polymerase.

FIG. 17B shows the same data as FIG. 17A, split into error types (Deletions, Insertions, Substitutions). PLATINUM™ SUPERFI™ II polymerase is shown to have similar positive effect on all error types. The overall deletion rate change is 40.4±55.1% (1/1157±840 bps to 1/1429±547 bps). The overall insertion rate change is 41.9±90.6% (1/2875±1201 bps to 1/3803±2841 bps). The overall substitution rate change is 32.7±21.2% (1/666±115 bps to 1/873±152 bps).

FIG. 17C shows data derived from twenty-five nucleic acid molecules assembled using PHUSION™ (“before”) or PLATINUM™ SUPERFI™ II (“after”) DNA polymerase and TkoEndoMS (“after”). These twenty-five fragments were different from the thirty fragments used to generate the data set out in FIG. 17A and FIG. 17B. This figure shows relative change in error rate for individual fragments after vs. before. The actual error rates and standard deviations for the individual fragments are 1 in 332±68 bp for before and 1 in 534±161 bp after, or an average improvement in error rate of 60.3±32.9%. Addition of TkoEndoMS is shown to result in further improvement of error rates.

FIG. 17D shows the same data as FIG. 17C, split into error types (Deletions, Insertions, Substitutions). Addition of TkoEndoMS is shown to increase the positive effect on insertions and substitutions. The overall deletion change rate is 44.4±51.3% (1/1019±261 bps to 1/1397±392 bps). The overall insertion change rate is 78.3±109.7% (1/2690±1191 bps to 1/4075±1517 bps). The overall substitution change rate is 77.6±36.5% (1/681±150 bps to 1/1217±380 bps).

### DETAILED DESCRIPTION

### Definitions

The term “nucleic acid molecule”, as used herein, refers to a covalently linked sequence of nucleotides or bases (e.g., ribonucleotides for RNA and deoxyribonucleotides for DNA but also include DNA/RNA hybrids where the DNA is in separate strands or in the same strands) in which the 3′ position of the pentose of one nucleotide is joined by a phosphodiester linkage to the 5′ position of the pentose of the next nucleotide. Nucleic acid molecules may be single- or double-stranded or partially double-stranded. Nucleic acid molecule may appear in linear or circularized form in a supercoiled or relaxed formation with blunt or sticky ends and may contain “nicks”. Nucleic acid molecules may be composed of completely complementary single strands or of partially complementary single strands forming at least one mismatch of bases. Nucleic acid molecules may further comprise two self-complementary sequences that may form a double-stranded stem region, optionally separated at one end by a loop sequence. The two regions of nucleic acid molecules which comprise the double-stranded stem region are substantially complementary to each other, resulting in self-hybridization. However, the stem can include one or more mismatches, insertions or deletions.

Nucleic acid molecules may comprise chemically, enzymatically, or metabolically modified forms of nucleotides or combinations thereof. Chemically synthesized nucleic acid molecules may refer to nucleic acids typically less than or equal to 200 nucleotides long (e.g., between 5 and 200, between 10 and 150, between 15 and 100, or between 20 and 50 nucleotides in length), whereas enzymatically synthesized nucleic acid molecules may encompass smaller as well as larger nucleic acid molecules as described elsewhere herein. Enzymatic synthesis of nucleic acid molecules may include stepwise processes using enzymes such as polymerases, ligases, exonucleases, endonucleases, recombinases or the like or a combination thereof. Thus, provided herein, in part, are compositions and combined methods relating to the enzymatic assembly of chemically synthesized nucleic acid molecules.

A nucleic acid molecule has a “5′-terminus” and a “3′-terminus” because nucleic acid molecule phosphodiester linkages occur between the 5′ carbon and 3′ carbon of the pentose ring of the substituent mononucleotides. The end of a nucleic acid molecule at which a new linkage would be to a 5′ carbon is its 5′ terminal nucleotide. The end of a nucleic acid molecule at which a new linkage would be to a 3′ carbon is its 3′ terminal nucleotide. A terminal nucleotide or base, as used herein, is the nucleotide at the end position of the 3′- or 5′-terminus. A nucleic acid molecule region, even if internal to a larger nucleic acid molecule (e.g., a sequence region within a nucleic acid molecule), also can be said to have 5′- and 3′-ends. Nucleic acid molecule also refers to short nucleic acid molecules, often referred to as, for example, primers or probes. Also, the terms “5′-” and “3′-” refer to strands of nucleic acid molecules. Thus, a linear, single-stranded nucleic acid molecule will have a 5′-terminus and a 3′-terminus. However, a linear, double-stranded nucleic acid molecule will have a 5′-terminus and a 3′-terminus for each strand. Thus, for nucleic acid molecules that encode proteins, for example, the 3′-terminus of the sense strand may be referred to.

The term “oligonucleotide”, as used herein, refers to DNA and RNA, and to any other type of nucleic acid molecule that is an N-glycoside of a purine or pyrimidine base but will typically be DNA. Oligonucleotides are thus a subset of nucleic acid molecules and may be single-stranded or double-stranded. Oligonucleotides (including primers as described below) may be referred to as “forward” or “reverse” to indicate the direction in relation to a given nucleic acid sequence. For example, a forward oligonucleotide may represent a portion of a sequence of the first strand of a nucleic acid molecule (e.g., the “sense” strand), whereas a reverse oligonucleotide may represent a portion of a sequence of the second strand (e.g., “antisense” strand) of said nucleic acid molecule or vice versa. In many instances, a set of oligonucleotides used to assemble longer nucleic acid molecules will comprise both forward and reverse oligonucleotides capable of hybridizing to each other via complementary regions. Oligonucleotides are typically less than 200 nucleotides, more typically less than 100 nucleotides in length. Thus, “primers” will generally fall into the category of oligonucleotide. Oligonucleotides can be prepared by any suitable method, including direct chemical synthesis by a method such as the phosphotriester method of Narang et al., Meth. Enzymol. 68:90-99 (1979); the phosphodiester method of Brown et al., Meth. Enzymol. 68:109-151 (1979); the diethylphosphoramidite method of Beaucage et al., Tetrahedron Letters 22:1859-1862 (1981); and the solid support method of U.S. Pat. No. 4,458,066. A review of synthesis methods of conjugates of oligonucleotides and modified nucleotides is provided in Goodchild, Bioconjugate Chemistry 1:165-187 (1990). Where appropriate, the term oligonucleotide may refer to a primer or probe and these terms may be exchangeably used herein.

Term “primer”, as used herein, refers to a short nucleic acid molecule capable of acting as a point of initiation of nucleic acid synthesis under suitable conditions. Such conditions include those in which synthesis of a primer extension product complementary to a nucleic acid strand is induced in the presence of different nucleoside triphosphates (e.g., A, C, G, T and/or U) and an agent for extension (for example, a DNA polymerase or reverse transcriptase) in an appropriate buffer and at a suitable temperature. A primer is generally composed of single-stranded DNA but can be provided as a double-stranded molecule for specific applications (e.g., blunt end ligation). Optionally, a primer can be naturally occurring or synthesized using chemical synthesis of recombinant procedures. The appropriate length of a primer depends on the intended use of the primer but typically ranges from about 6 to about 200 nucleotides, including intermediate ranges, such as from about 10 to about 50 nucleotides, from about 15 to about 35 nucleotides, from about 18 to about 75 nucleotides and from about 25 to about 150 nucleotides. The design of suitable primers for the amplification of a given target sequence is well known in the art and described in the literature (see for example OLIGOPERFECT™ Designer, Thermo Fisher Scientific). Primers can incorporate additional features which allow for the detection or immobilization of the primer but do not alter the basic property of the primer, that of acting as a point of initiation of DNA synthesis. Thus, a primer may include a detectable moiety or label. For example, the label can include fluorescent, luminescent or radioactive moieties.

A set of primers used in the same amplification reaction may have melting temperatures that are substantially the same, where the melting temperatures are within about 10-5° C. of each other, or within about 5-2° C. of each other, or within about 2-0.5° C. of each other, or less than about 0.5° C. of each other.

The terms “complementary” or “complementarity”, as used herein, refer to the natural binding of nucleic acid molecules (primers, oligonucleotides or polynucleotides etc.) under permissive salt and temperature conditions by base pairing. For example, the sequence “A-G-T” binds to the complementary sequence “T-C-A.” Complementarity between two single-stranded molecules may be “partial,” such that only some of the nucleic acids bind, or it may be “complete,” such that total complementarity exists between the single-stranded molecules. The degree of complementarity between nucleic acid strands has significant effects on the efficiency and strength of the hybridization between the nucleic acid strands. This is of particular importance in amplification reactions, which depend upon binding between nucleic acids strands. Complementary regions between nucleic acid molecules such as oligonucleotides may also be referred to as “overlaps” or “overlapping” regions as defined below.

The term “hybridization”, as used herein, refers to any process by which a strand of nucleic acid binds with a complementary strand through base pairing. Hybridization and the strength of hybridization (for example, the strength of the association between the nucleic acids) is impacted by such factors as the degree of complementary between the nucleic acids, stringency of the conditions involved, the Tₘ of the formed hybrid, and the G:C ratio within the nucleic acids.

The term “homologous”, as used herein, refers to a degree of complementarity. Nucleic acid sequences may be partially or completely homologous (identical). A partially complementary sequence is one that at least partially inhibits a completely complementary sequence from hybridizing to a target nucleic acid and is referred to using the functional term “substantially homologous”.

The term “overlap” or “overlapping”, as used herein, refers to a sequence homology or sequence identity shared by a portion of two or more oligonucleotides.

The term “gene” or “gene sequence”, as used herein, generally refers to a nucleic acid sequence that encodes a discrete cellular product. In many instances, a gene or gene sequence includes a DNA sequence that comprises an open reading frame (ORF) and can be transcribed into mRNA which can be translated into polypeptide chains, transcribed into rRNA or tRNA or serve as recognition sites for enzymes and other proteins involved in DNA replication, transcription and regulation. These genes include, but are not limited to, structural genes, immunity genes, regulatory genes and secretory (transport) genes etc. However, as used herein, “gene” refers not only to the nucleotide sequence encoding a specific protein, but also to any adjacent 5′ and 3′ non-coding nucleotide sequence involved in the regulation of expression of the protein encoded by the gene of interest. These non-coding sequences include terminator sequences, promoter sequences, upstream activator sequences, regulatory protein binding sequences, and the like. In many instances, a gene is assembled from shorter oligonucleotides or nucleic acid fragments.

The terms “fragment”, “subfragment”, “segment”, or “component” or similar terms, as used herein, in connection with a nucleic acid molecule or sequence either refer to a product or intermediate product obtained from one or more process steps (e.g., synthesis, assembly PCR, amplification etc.), or refer to a portion, part or template of a longer or modified nucleic acid product to be obtained by one or more process steps (e.g., assembly PCR, amplification, ligation, cloning etc.). In some instances, a nucleic acid fragment or subfragment may represent both, an assembly product (e.g., assembled from multiple oligonucleotides) and a starting compound for higher order assembly (e.g., a gene assembled from multiple fragments or a fragment assembled from multiple subfragments etc.).

As used herein, “amines” or “amine compound”, as used herein, includes chemicals of Formula I, immediately below, or salts thereof:

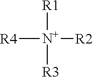

wherein R1 is H; R2 is chosen from alkyl, alkenyl, alkynyl, or (CH₂)n-R5, wherein n=1 to 3, and R5 is aryl, amino, thiol, mercaptan, phosphate, hydroxy, alkoxy; and R3 and R4 may be the same or different and are independently chosen from H or alkyl, with the proviso that if R2 is (CH₂)n-R5, then at least one of R3 and/or R4 is alkyl. As such, amines include diethylamine hydrochloride, diisopropylamine hydrochloride, ethyl(methyl)amine hydrochloride, trimethylamine hydrochloride, and dimethylamine hydrochloride.

The term “vector”, as used herein, refers to any nucleic acid molecule capable of transferring genetic material into a host organism. The vector may be linear or circular in topology and includes but is not limited to plasmids, viruses, bacteriophages. The vector may include amplification genes, enhancers or selection markers and may or may not be integrated into the genome of the host organism.

The term “plasmid”, as used herein, refers to a vector that can be genetically modified to insert one or more nucleic acid molecules (e.g., assembly products). Plasmids will typically contain one or more region that renders it capable of replication in at least one cell type.

The term “amplification”, as used herein, relates to the production of additional copies of a nucleic acid molecule. Amplification is often carried out using polymerase chain reaction (PCR) technologies well known in the art (see, e.g., Dieffenbach, C. W. and G. S. Dveksler (1995) PCR Primer, a Laboratory Manual, Cold Spring Harbor Press, Plainview, N.Y.) but may also be carried out by other means including isothermal amplification methods such as, e.g., transcription mediated amplification, strand displacement amplification, rolling circle amplification, loop-mediated isothermal amplification, helicase-dependent amplification, single primer isothermal amplification or recombinase polymerase amplification (see, e.g., Fakruddin et al., “Nucleic acid amplification: Alternative methods of polymerase chain reaction”, J. Pharm Bioallied Sci, 2013, v.5(4), 245-252; or Gill and Ghaemi, “Nucleic acid isothermal amplification technologies: a review”, Nucleosides Nucleotides Nucleic Acids. 2008 27(3), 224-43). Amplification reactions may be carried out using terminal primers to reconstruct each strand of a denatured double-stranded nucleic acid molecules.

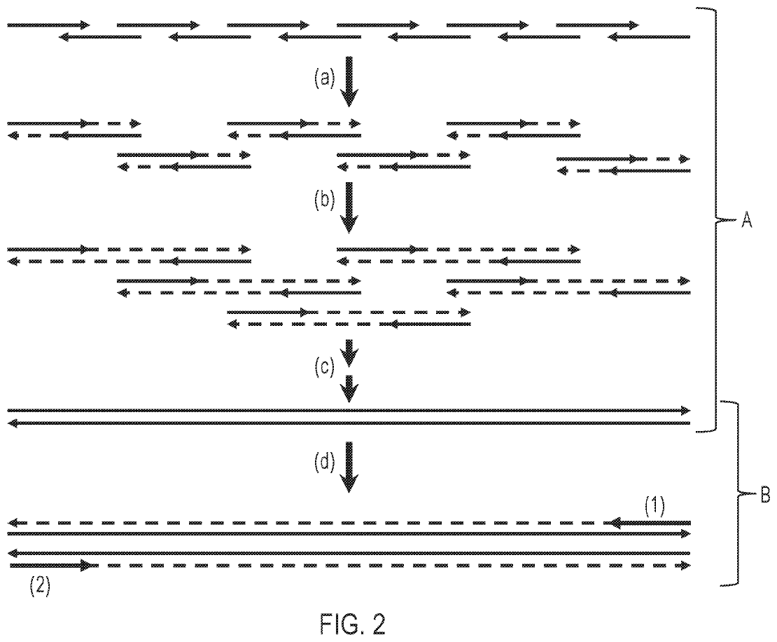

The term “assembly chain reaction”, also referred to herein as “assembly PCR”, when used herein, refers to the assembly of larger nucleic acid molecules from smaller nucleic acid molecules by polymerase mediated extensions of overlapping, partially complementary nucleic acid molecules. The overlapping, partially complementary nucleic acid molecules may be single-stranded or double-stranded. Further, double-stranded nucleic acid molecules will typically be denatured before or as port of use in an assembly chain reaction. An example of an assembly chain reaction is set out at the top of FIG. 2, where overlapping, partially complementary nucleic acid molecules are used to generate large nucleic acid molecules with each polymerase mediation extension step.

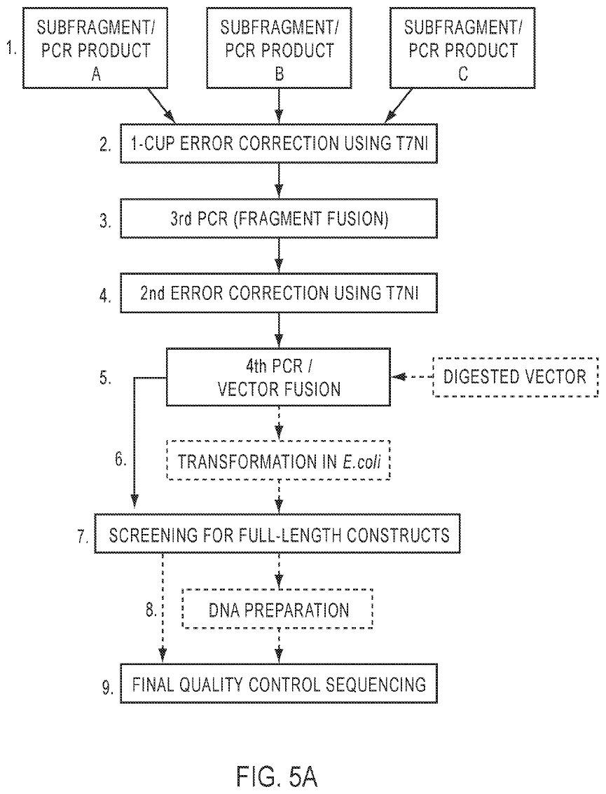

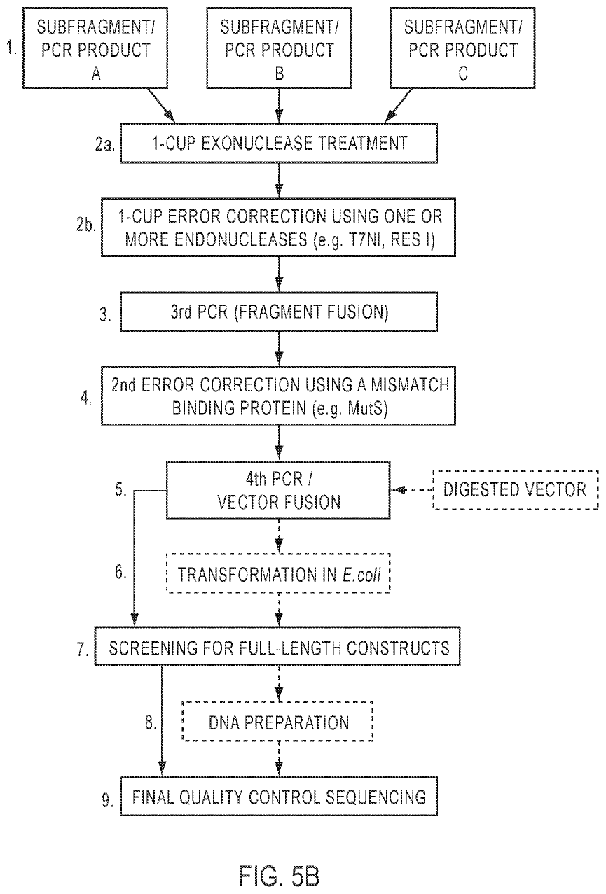

The term “post-primary amplification error correction”, as used herein, refers to the amplification-based error correction steps that occur after the end of the workflow shown in FIG. 2. In the workflow of FIG. 2, oligonucleotides are first assembled (primary assembly PCR), then amplified using terminal primers (primary amplification). Once this has occurred, additional rounds of error correction (e.g., error correction involving PCR-based fragment assembly and amplification) may occur. For example, if in the workflow of FIG. 5A, the three Subfragment/PCR products of step 1 were made using the workflow of FIG. 2, then all error correction steps in FIG. 5A would be post-primary amplification error corrections.

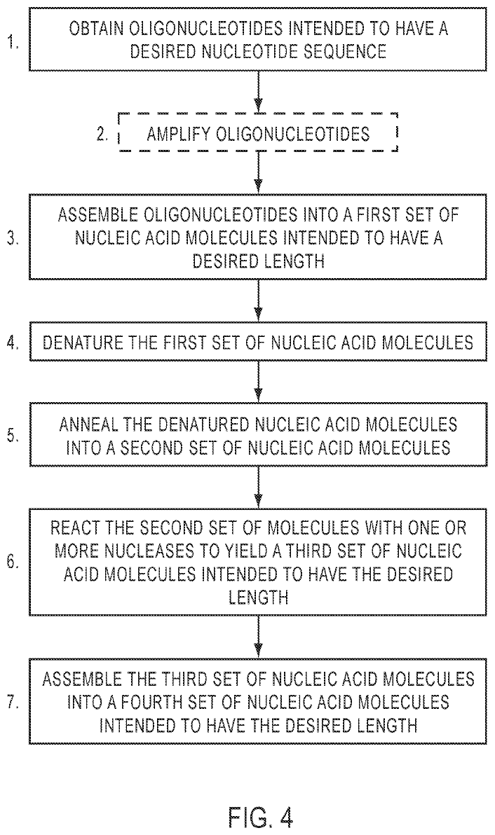

Error correction will often involve the use of a mismatch endonuclease. An exemplary error correction process is set out in FIG. 4. In this figure, double-stranded nucleic acid molecules assembled from amplified oligonucleotides are denatured then reannealed (lines 4 and 5). The reannealed nucleic acid molecules some of which may contain one or more mismatches are then contacted with, for example, a mismatch endonuclease (line 6) to cleave the nucleic acid molecules at or nearby the sites of mismatch. The cleaved nucleic acid molecules in the reaction mixture of line 6 are then re-assembled by overlap extension PCR and amplified to yield error-free nucleic acid molecules (output of the process in line 7) intended to be of the same length as the “uncorrected” starting nucleic acid molecules (line 3).

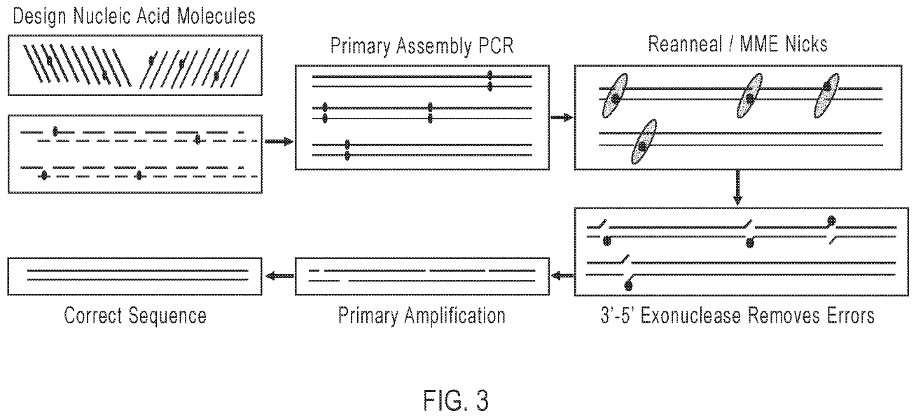

The term “non-amplification error correction”, as used herein, refers to error correction processes that do not involve nucleic acid amplification. An example of such a method is one where nucleic acid strands are hybridized to each other, followed by removal of double-stranded nucleic acid molecule containing mismatches using mismatch binding proteins (see, e.g., FIG. 3).

The term “adjacent”, as used herein, refers to a position in a nucleic acid molecule immediately 5′ or 3′ to a reference region.

The term “sequence fidelity”, as used herein, refers to the level of sequence identity of a nucleic acid molecule as compared to a reference sequence. Full identity being 100% identical over the full-length of the nucleic acid molecules being scored for sequence identity. Sequence fidelity can be measure in a number of ways, for example, by the comparison of the actual nucleotide sequence of a nucleic acid molecule to a desired nucleotide sequence (e.g., a nucleotide sequence that one wishes to be used to generate a nucleic acid molecule). Another way sequence fidelity can be measured is by comparison of sequences of two nucleic acid molecules in a reaction mixture. In many instances, the difference on a per base basis will be, on average, the same.

The error rates for DNA polymerases can be measured by the quantification of total errors or different types of errors. With respect to high fidelity DNA polymerases as set out herein, the error rate “benchmark” is set based upon the substitution rate. In particular, a high fidelity DNA polymerase will exhibit a substitution error rate that is lower of 1.0×10⁻⁵ substitution per base. Examples of high fidelity polymerase include PHUSION™ DNA polymerase, PLATINUM™ SUPERFI™ II DNA polymerase, Q5® DNA Polymerase, and PRIMESTAR® GXL DNA Polymerase (Takara). Methods for determining error rate are known in the art and are set out for example, in Potapov et al., “Examining Sources of Error in PCR by Single Molecule Sequencing”, PLOS ONE, DOI:10.1371/journal.pone.0169774 Jan. 6, 2017.

The term “transition”, when used in reference to the nucleotide sequence of a nucleic acid molecule, refers to a point mutation that changes a purine nucleotide to another purine (A ↔G) or a pyrimidine nucleotide to another pyrimidine (C↔T).

The term “transversion”, when used in reference to the nucleotide sequence of a nucleic acid molecule, refers to a point mutation involving the substitution of a (two ring) purine for a (one ring) pyrimidine or a (one ring) pyrimidine for a (two ring) purine.

The term “indel”, as used herein, refers to the insertion or deletion of one or more bases in a nucleic acid molecule.

The term “mismatch”, as used herein, refers to two bases in different strands of a double-stranded nucleic acid molecule that do not form Watson-Crick base pairing, while surrounding bases in of different nucleic acid strands have sequence complementarity and do form Watson-Crick base pairing bases. The length of the complementary regions may vary but with often be of at least twenty base pairs. With respect to each strand of a nucleic acid molecule which contains only the four standard DNA bases, there are four correct (Watson-Crick base pairing) complementary matches (i.e., A/T, T/A/G/C, and C/G) and twelve “mismatches” (i.e., A/A, A/C, A/G, T/T, T/C, T/G, G/G, G/A, G/T, C/C, C/T, and C/A). With respect to base pairing, in the absence of strand reference, there are two correct complementary matches (i.e., A/T and G/C) and eight “mismatches” (i.e., A/A, A/C, A/G, T/T, T/C, T/G, G/G, and C/C). In terms of substitutions, these mismatches can be expressed as (1) A to G and T to C, (2) G to A and C to T, (3) A to C and T to G, (4) A to T and T to A, (5) G to C and C to G, and (6) G to T and C to A.

The term “thermostable”, as used herein in reference to protein refers to a protein that retains at least 85% the protein biological activity after heating to 95° C. for 5 minutes. Thermostable proteins may or may not have biological activity at 95° C. Thus, depending on the protein, an assay of retained biological activity may be performed after incubation at 95° C. for 5 minutes or at another (e.g., lower) temperature, using as a “benchmark” of the same protein not heated to 95° C. for 5 minutes.

The term “mismatch recognition protein”, as used herein, refers to a protein with specific biological activity for mismatched bases in double-stranded DNA. These activities may include nuclease activity and/or binding activity. Such proteins include resolvases, MutS and MutS homologs, MutM and MutM homologs, MutY and MutY homologs, and members of the RecB nuclease family of proteins. Mismatch binding proteins and mismatch endonucleases are both mismatch recognition proteins. Mismatch recognition proteins may be thermostable or non-thermostable. Some exemplary mismatch recognition proteins are set out in Table 15, as well as other tables provided herein.

The term “mismatch endonuclease” or “MME” (also referred to as a “mismatch repair endonuclease”), as used herein refers to a nuclease having the activity of cleaving (one or both strands) of double-stranded nucleic acid molecules at or near (e.g., within from about one to about five base pairs) mismatch sites. Mismatch endonuclease activity includes the ability to cleave phosphodiester bonds at or near nucleotides forming mismatched base pairs, and an activity of cleaving phosphodiester bonds adjacent to nucleotides located 1 to 5, often 1 to 3 base pairs away from mismatched base pairs. Examples of proteins with mismatch endonuclease activity are set out below in Tables 13 and 15. Specific examples of mismatch endonucleases include as CEL I (Till et al., Nucl. Acid Res. 32:2632-2641 (2004)) and CEL II (U.S. Pat. No. 7,129,075), bacteriophage resolvases, such as T7NI and T4 endonucleases VII (Mashal, et al., Nature Genetics 9:177-183 (1995)), E. coli Endonuclease V (Yao and Kow, J. Biol. Chem. 272:30774-30779 (1997)), TkoEndoMS (Ishino et al., Nucl. Acids Res. 44:2977-2986 (2016)), and Pyrococcus furiosus EndoMS (referred to herein as “PfuEndoMS”). Mismatch endonucleases may be thermostable (TsMME) or non-thermostable.

The term “EndoMS”, as used herein, refers to mismatch specific endonucleases that share at least 50% amino acid sequence identity with one or more of the EndoMS proteins set out in Table 15 and have mismatch specific endonuclease activity. “Nucs” has been used in the art as an alternative term for EndoMS. Thus, the terms “EndoMS” and “Nucs” may be used interchangeably.

The term “mismatch binding protein” (also referred to as a “mismatch repair binding protein”), as used herein refers to a protein with specific binding activity for mismatched bases in double-stranded DNA. Examples of such proteins are set out below in Tables 12 and 15. Many of these proteins are MutS homologs. Mismatch binding proteins may be thermostable or non-thermostable.

The term “error correction”, as used herein refers to processes designed to a decrease the total number nucleotide sequence defects in nucleic acid molecules of a population. These defects can be mismatches, insertions, deletions and/or substitutions. Defects can occur when nucleic acid molecules generated (e.g., by chemical or enzymatic synthesis) are each intended to contain a particular base at a location, but a different base is present at that location in one or more nucleic acid molecules.

An exemplification of error correction is as follows. Assume that there is a population of double-stranded nucleic acid molecules have a desired length of 100 base pairs. Also, assume that the two strands of the double-stranded nucleic acid molecules were each synthesized separately and the hybridized to each other to form double-stranded nucleic acid molecules of the population. Further assume that nucleic acid synthesis results in an average of 1 error per 200 nucleotides. In such an instance, there would be 1 “error” per 100 base pairs. Thus, on average, each double-stranded nucleic acid molecule of the population would contain one error. Of course, some of the double-stranded nucleic acid molecules in the population would have no errors and other double-stranded nucleic acid molecules would have more than one error. If an error correction process removed half of the nucleic acid molecules from the population and none of the nucleic acid molecules without errors were removed, then the error rate in the remaining double-stranded nucleic acid molecules in the population would be less than 1 in 200 base pairs. This is so because, as suggested above, some of the removed nucleic acid molecules would have more than one error and none of the “correct” nucleic acid molecules were removed.

As used herein, the phases “error correction round” and “round of error correction” refers to a series of steps that result in the cleavage or removal of nucleic acid molecules with errors from a population of nucleic acid molecules. Using FIG. 4 for purposes of illustration, lines 4-7 set out one round of error correction. The process set out in FIG. 4 involves a series of amplification reactions (e.g., PCR cycles) but rounds of error correction do not necessarily require this. For example, a modification of the process set out in FIG. 4 is where a mismatch binding protein may be used to separate nucleic acid molecules with mismatches (see line 5) from nucleic acid molecules that do not have mismatches.

As used herein, an “error reducing polymerase reagent” is a composition which comprises a polymerase (e.g., a DNA polymerase) and an additional component that reduces the number of errors in amplified nucleic acid molecules (e.g., by from about 5% to about 30%, from about 5% to about 30%, from about 5% to about 30%, from about 10% to about 40%, from about 10% to about 70%, etc.), wherein the additional component is not a mismatch recognition protein. One category of such compounds are amines, such as amines set out herein.

The term “transformation”, as used herein, describes a process by which an exogenous nucleic acid molecule enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted nucleic acid is capable of replication either as an autonomously replicating plasmid or as part of the host chromosome. They also include cells that transiently express the inserted DNA or RNA for limited periods of time.

The term “solid support”, as used herein refers to a porous or non-porous material on which polymers such as oligonucleotides or nucleic acid molecules can be synthesized and/or immobilized. As used herein “porous” means that the material contains pores which may be of non-uniform or uniform diameters (for example in the nm range). Porous materials include paper, synthetic filters etc. In such porous materials, the reaction may take place within the pores. The solid support can have any one of a number of shapes, such as pin, strip, plate, disk, rod, fiber, bends, cylindrical structure, planar surface, concave or convex surface or a capillary or column. The solid support can be a particle, including bead, microparticles, nanoparticles and the like. The solid support can be a non-bead type particle (e.g., a filament) of similar size. The support can have variable widths and sizes. For example, sizes of a bead (e.g., a magnetic bead) which may be used in the practice of aspects of methods set out herein may vary widely but include beads with diameters between 0.01 μm and 100 μm, 0.005 μm and 100 μm, 0.005 μm and 10 μm, 0.01 μm and 100 μm, 0.01 μm and 1,000 μm, between 1.0 μm and 2.0 m, between 1.0 μm and 100 μm, 15 between 2.0 μm and 100 μm, between 3.0 μm and 100 μm, between 0.5 μm and 50 μm, between 0.5 μm and 20 μm, between 1.0 μm and 10 μm, between 1.0 μm and 20 μm, between 1.0 μm and 30 μm, between 10 μm and 40 μm, between 10 m and 60 μm, between 10 μm and 80 μm, or between 0.5 μm and 10 μm.

The support can be hydrophobic or capable of binding a molecule via hydrophobic interaction. The support can be hydrophilic or capable of being rendered hydrophilic and includes inorganic powders such as silica, magnesium sulfate, and alumina; natural polymeric materials, particularly cellulosic materials and materials derived from cellulose, such as fiber containing papers such as filter paper, chromatographic paper or the like. The support can be immobilized at an addressable position of a carrier such as, e.g., a multiwell plate or a microchip. The support can be loose or particulate (such as, e.g., a resin material or a bead in a well) or can be reversibly immobilized or linked to the carrier (e.g., by cleavable chemical bonds or magnetic forces etc.). In some aspects, solid support may be fragmentable. Solid supports may be synthetic or modified naturally occurring polymers, such as nitrocellulose, carbon, cellulose acetate, polyvinyl chloride, polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), polyvinylidene difluoride (PVDF) membrane, glass, controlled pore glass, magnetic controlled pore glass, magnetic or non-magnetic beads, ceramics, metals, and the like; either used by themselves or in conjunction with other materials. In some aspects, the support can be in a chip, array, microarray or microwell plate format. In many instances, a support used in methods or compositions of set out herein will be one where individual nucleic acid molecules are synthesized on separate or discrete areas to generate features (i.e., locations containing individual nucleic acid molecules) on the support. In some aspects, the size of the defined feature is chosen to allow formation of a microvolume droplet or reaction volume on the feature, each droplet or reaction volume being kept separate from each other. As described herein, features are typically, but need not be, separated by interfeature spaces to ensure that droplets or reaction volumes or between two adjacent features do not merge. Interfeatures will typically not carry any nucleic acid molecules on their surface and will correspond to inert space. In some aspects, features and interfeatures may differ in their hydrophilicity or hydrophobicity properties. In some aspects, features and interfeatures may comprise a modifier. In some instances set out herein, the feature is a well or microwell or a notch. Nucleic acid molecules may be covalently or non-covalently attached to the surface or deposited or synthesized or assembled on the surface.

“a”, “an”, and “the” include plural reference unless the context clearly dictates otherwise.

### Overview

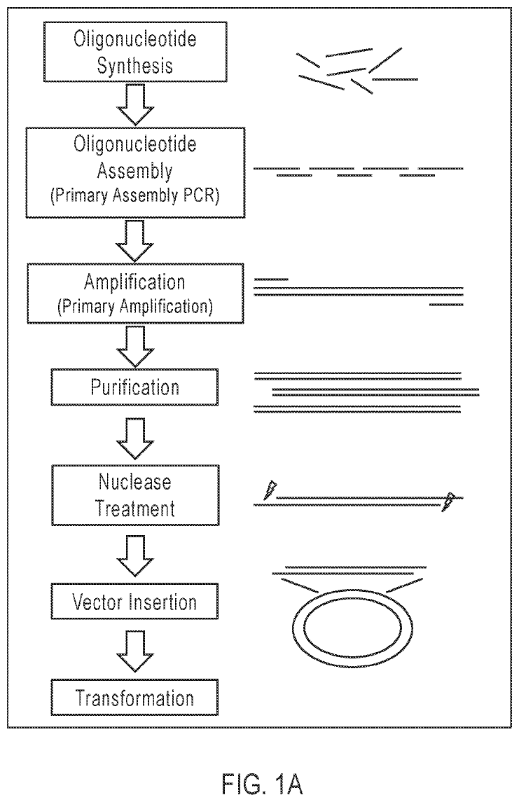

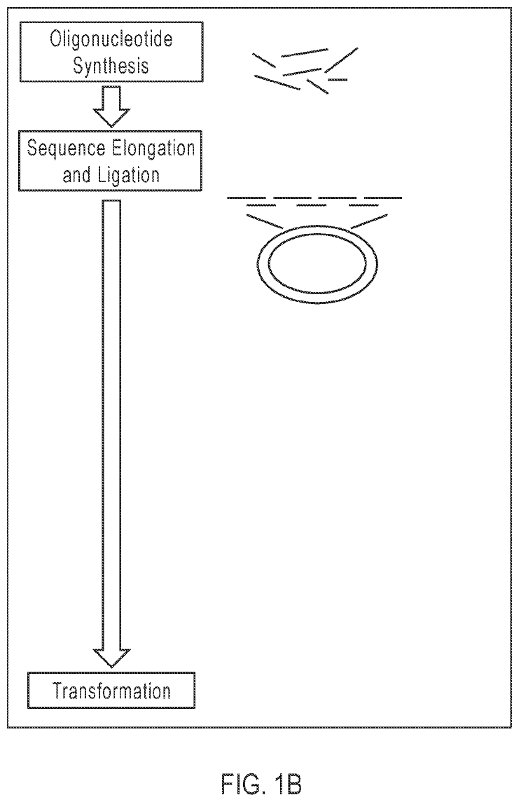

Compositions and methods set out herein are directed, in part, to the preparation of nucleic acid molecules having high sequence fidelity. While numerous aspects and variations may be employed, in many instances, nucleic acid molecules will be synthesized (e.g., chemically, enzymatically, etc.). These synthesized nucleic acid molecules may, optionally, then be assembled to form one or more larger nucleic acid molecules by, for example, assembly PCR (e.g., primary assembly PCR). FIG. 1A and FIG. 1B are schematics showing exemplary assembly PCR steps that may be used in methods set out herein.

There is generally relatively low abundance and the semi-random distribution of sequence errors in synthesized oligonucleotides. In many instances, when nucleic acid molecules with erroneous bases (e.g., deletions, insertion, substitutions) hybridize with nucleic acid molecules with correct bases, a region is formed that does not exhibit standard Watson-Crick base pairing. These “non-standard” regions may be used for recognition of nucleic acid molecules that contain errors. Further, once these “non-standard” regions are detected in a population of nucleic acid molecules, nucleic acid molecules containing these regions may be removed from the population or they may be modified in such a way as to prevent their amplification or low their ability to be amplified.

A number of methods may be used to reduce the percentage of nucleic acid molecules containing errors (e.g., deletions, insertion, substitutions) in a population. These methods include:
1. Cleavage of nucleic acid molecules that contain errors,
2. Separation of nucleic acid molecules that contain error from nucleic acid molecules that do not contain errors, and
3. Suppressing/inhibiting the amplification of nucleic acid molecules that contain error as compared to nucleic acid molecules that do not contain errors.

Further, two or more of the above methods may be used to reduce the number of errors present in nucleic acid molecules.

Much of the disclosure set out herein is directed to compositions and methods for the synthesis, assembly (e.g., assembly PCR) and amplification of nucleic acid molecules. Provided herein are compositions and methods for the generation of nucleic acid molecules with high sequence fidelity.

For some applications, it is important to use of nucleic acid molecules with low error rates. For purposes of illustration, consider the situation where one hundred nucleic acid molecules are to be assembled, each molecule is one hundred base pairs in length and there is one error per 200 base pairs. The net result is that there will be, on average, 50 sequence errors in each 10,000 base pair assembled nucleic acid molecule. If one intends, for example, to express one or more proteins from the assembled nucleic acid molecule, then the number of amino acid sequence errors would likely be considered to be too high. Further, a number of the protein coding region nucleotide sequence errors will result in “frame shifts” mutations yielding proteins that will generally not be desired. Also, non-frame shift coding regions may result in the formation of proteins with point mutations. All of these will “dilute the purity” of the desired protein expression product and many of the produced “contaminant” proteins will be carried over into the final expression product mixture even if affinity purification is employed.

High sequence fidelity can be achieved by several means, including sequencing of nucleic acid fragments prior to assembly or partially assembled nucleic acid molecules, sequencing of fully assembled nucleic acid molecules to identify ones with correct sequences, and/or error correction.

Errors may find their way into nucleic acid molecules in a number of ways. Examples of such ways include chemical synthesis errors, amplification/polymerase mediated errors (especially when non-proofreading polymerases are used), and assembly PCR mediated errors (usually occurring at nucleic acid fragment junctions).

Sequence errors in nucleic acid molecules may be referenced in a number of ways. As examples, there is the error rate associated with the synthesis nucleic acid molecules, the error rate associated with nucleic acid molecules after error correct and/or the selection, and the error rate associated with end product nucleic acid molecules (e.g., error rates of (1) a synthetic nucleic acid molecules that have either been selected for the correct sequence or (2) assembled chemically synthesized nucleic acid molecules). These errors may come from the chemical synthesis process, assembly processes, and/or amplification processes. Errors may be removed or prevented by methods, such as, the selection of nucleic acid molecules having correct sequences, error correction, and/or improved chemical synthesis methods.

In some instances, methods set out herein may combine error removal and prevention methods to produce nucleic acid molecules with relative low numbers of errors. Thus, assembled nucleic acid molecules produced by methods of set out herein may have error rates from about 1 base in 1,500 to about 1 base in 30,000, from about 1 base in 2,000 to about 1 base in 30,000, from about 1 base in 4,000 to about 1 base in 30,000, from about 1 base in 8,000 to about 1 base in 30,000, from about 1 base in 10,000 to about 1 base in 30,000, from about 1 base in 15,000 to about 1 base in 30,000, from about 1 base in 10,000 to about 1 base in 20,000, etc.

Two ways to lower the number of errors in assembled nucleic acid molecules is by (1) selection of nucleic acid molecules (e.g., oligonucleotides, subfragments, etc.) for assembly with corrects sequences and (2) correction of errors in nucleic acid molecules, partially assembled sub-assemblies, or fully assembled nucleic acid molecules.

Errors may be incorporated into nucleic acid molecules regardless of the method by which the nucleic acid molecules are generated. Even when nucleic acid molecules known to have correct sequences are used for assembly PCR, errors can find their way into the final assembly products. Thus, in many instances, error reduction will be desirable.

In many instances, regardless of the method by which a larger nucleic acid molecule is generated from chemically synthesized oligonucleotides, errors from the chemical synthesis process will be present. While sequencing of individual nucleic acid molecules may be performed to identify and select error-free nucleic acid molecules, alternative approaches may comprise one or more error correction or removal steps. Thus, in many instances, error correction will be desirable. Error correction can be achieved by any number of means. Typically, such error removal steps will be performed after a first round of assembly PCR. Thus, in some aspects, methods set out herein may involve the following (in this order or different orders): (i) fragment amplification and/or assembly PCR (e.g., according to the methods described herein), (ii) error correction, (iii) final assembly (e.g., according to the in vitro or in vivo methods described herein, e.g., using a protocol as illustrated in FIG. 1A or 1B).

Errors may be removed from nucleic acid molecules or otherwise avoided at one or more locations in workflows used to generate these molecules. Using the workflow set out in FIG. 1A for purposes of illustration, oligonucleotide synthesis may be performed under conditions where few sequence errors are introduced. Nucleic acid assembly PCR (e.g., oligonucleotide assembly) may be performed in conjunction with mismatch recognition-based error correction. Assembled nucleic acid molecules may be amplified in conjunction with mismatch recognition-based error correction. Once assembled, nucleic acid molecules may undergo mismatch recognition-based error correction in the absence of assembly PCR or amplification. This will often be done by heat denaturation of the subject nucleic acid molecules, followed by renaturation of the nucleic acid molecules which are then and contacted with one or more mismatch recognition protein.

Further, the introduction of errors into nucleic acid molecules can be avoided or lessened in a number of ways. Some of these ways include the use of nucleic acid starting materials that contain few errors. Set out in Example 2 and Tables 10 and 11, the use of nucleic acid starting materials that contain few errors results in fewer errors being present in assembled, error corrected molecules. This is believed to be due to error correction methods not always being able to correct 100% of errors present. Thus, in general, the fewer errors that are present for correction results in fewer errors after error correction.

In many instances, nucleic acid molecule starting material will have an initial average number of sequence errors that is from about 1 in 250 to about 1 in 2,000 (e.g., from about 1 in 250 to about 1 in 1,900, from about 1 in 250 to about 1 in 1,500, from about 1 in 250 to about 1 in 1,200, from about 1 in 250 to about 1 in 1,000, from about 1 in 250 to about 1 in 800, from about 1 in 400 to about 1 in 1,900, from about 1 in 400 to about 1 in 1,500, from about 1 in 400 to about 1 in 1,100, from about 1 in 650 to about 1 in 2,000, from about 1 in 650 to about 1 in 1,700, from about 1 in 650 to about 1 in 1,500, etc.).

As also set out in Example 2, to some extent, error correction efficiency various with thermocycling conditions used. Thus, one factor that may be changed to yield product nucleic acid molecules with low numbers of error is thermocycling conditions.

Another way to avoid the introduction of errors into nucleic acid molecules, for example, is by the use of synthesis methods to generate nucleic acid sub-units with few errors. Another way is to use high fidelity polymerases and high-fidelity amplification methods for low error replication assembly and amplification of nucleic acid molecules.

Using the workflow in FIG. 2 for purposes of illustration, synthetically produced oligonucleotides are assembled by a DNA polymerase through a series of heating and cooling steps, resulting in large nucleic acid molecules with each assembly PCR cycle. Hybridization of complementary regions of single-stranded nucleic acid molecules occur during each assembly PCR cycle. Regions that do not exhibit standard Watson-Crick base pairing can form during these hybridization reactions and, when this occurs, these resulting double-stranded nucleic acid molecules are “marked” as containing errors. Methods for generating “error corrected” populations of nucleic acid molecules set out herein employ DNA polymerases and mismatch recognition proteins for eliminating from or decreasing the prevalence of nucleic acid molecules containing errors from a mixed population (“error correction”).

Again using the workflow of FIG. 2 for purposes of illustration, error correction may be performed at any one or more steps, as well as other places in a larger workflow (e.g., after the primary amplification shown) and may include multiple error correction reagents and error correction mechanism, as well as other error reducing methods. Further, FIG. 2 shows a series of assembly PCR and amplification reactions. Error correction may occur in none, some or all of these steps. For example, FIG. 2 shows four overlap extension cycles of an assembly PCR reaction (based upon the number of downward pointing arrows (a)-(c) shown). When, for example, a thermostable mismatch recognition protein is used, it could be added prior to the first assembly PCR cycle or could be added during the assembly PCR reaction (i.e. after one or more of the extension cycles have been completed). Examples of error correcting reagents that may be used include mismatch endonucleases and mismatch binding proteins.

Reagents that may be used to perform error correcting include mismatch endonucleases, mismatch binding proteins, and high-fidelity polymerases and reagents that contain high fidelity polymerases. Further, proteins used in methods set out herein may be thermostable or non-thermostable. One example of a reagent that contains a high-fidelity polymerase is PLATINUM™ SUPERFI™ II DNA polymerase (Thermo Fisher Scientific, cat. no. 12361010).

One general workflow for error correction of nucleic acid molecules is where either single-stranded nucleic acid molecules with regions of sequence complementarity are hybridized to each other or double-stranded nucleic acid molecules are denatured and then hybridized to each other. In such instances, when two nucleic acid strands that differ in nucleotide sequence by one or more nucleotides hybridize to each other and the resulting double-stranded nucleic acid molecule will generally form a region where Watson-Crick base pairing is not exhibited. In some instances, error correction processes may be based upon recognition of regions where Watson-Crick base pairing is not exhibited. Thus, in many instances, error correction processes will involve the hybridization of single-stranded nucleic acid molecules to form double-stranded nucleic acid molecules. While error correction may be performed in the absence of a DNA polymerase, assembly PCR and amplification processes that may include error correction are shown in FIG. 1A, FIG. 1B and FIG. 2.

Methods set out herein include various combinations of error reduction, error correction related to assembly PCR and/or amplification steps. Further, error correction processes may be integrated into such steps or occur before or after such steps.

Methods set out herein may involve any number of steps and combinations of workflows set out herein. Using the workflow of FIG. 1A, FIG. 2, and FIGS. 5A and 5B to illustrate exemplary aspects of methods set out herein, oligonucleotides having termini of overlapping sequence complementarity may be generated (FIG. 1A). These oligonucleotides may then be assembled by a series of assembly PCR cycles in what is termed a primary assembly PCR (FIG. 1A and FIG. 2). The assembly products are then amplified using terminal primers in what is termed a primary amplification (FIG. 1A and FIG. 2). Assembly products generated in separate assembly PCR reactions which have complementary terminal sequences, as, for example, set out in FIG. 2, may be further assembled as shown at the top of FIGS. 5A and 5B in what would be termed a secondary assembly PCR. In these examples, subfragment PCR products A, B and C are combined into a vessel to perform a 1-cup mismatch cleavage-based error correction followed by a PCR step to fuse and extend the error-corrected fragments (referred to as 3^{rd} PCR in lines 3, respectively) the result of which are longer nucleic acid assembly products comprising fragments A, B and C. Error correction may occur in, before, and/or after each assembly and/or amplification step.

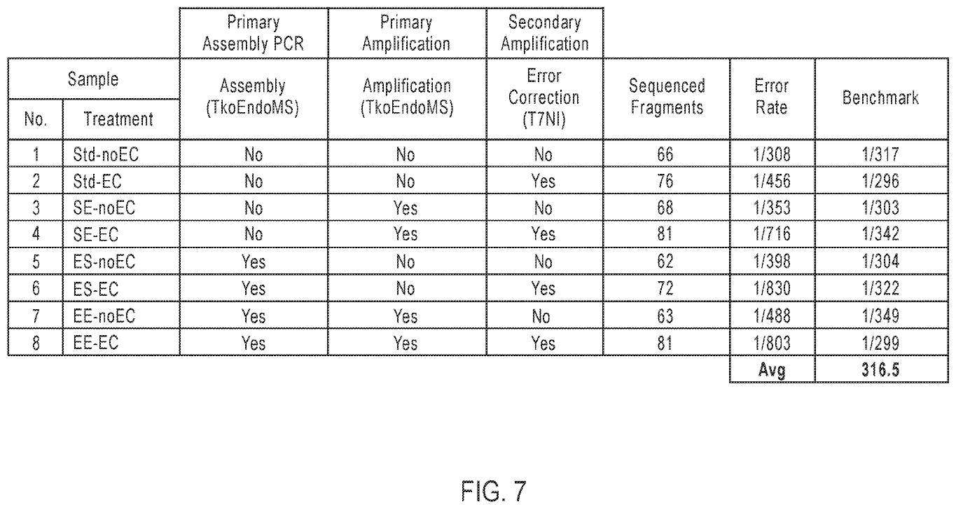

Using the data set out in FIG. 7 to illustrate, primary assembly PCRs were performed in the presence or absence of TkoEndoMS. In each instance, this was followed by primary amplification also in the presence or absence of TkoEndoMS. This was then followed by error correction using T7NI, which involves a secondary amplification.

FIG. 1B shows a workflow where only primary assembly PCR and primary amplification occur.

In summary, in some aspect, provided herein are methods comprising a combination of assembly PCR and/or amplification steps, where error correction may occur during or between any of such steps. In many instances, one or more thermostable mismatch recognition protein may be present during assembly PCR and/or amplification steps.

The term “primary assembly PCR” refers to an assembly PCR reaction where single-stranded nucleic acid molecules are assembled to form double-stranded nucleic acid molecules that are longer in length than the individual single-stranded nucleic acid molecules. Even though the workflow in FIG. 1B shows an assembly reaction where single-stranded nucleic acid molecules are assembled with a double-stranded nucleic acid molecule (i.e., a vector), this is considered to include primary assembly PCR because the vector insert is formed from single-stranded nucleic acid molecules. Thus, in such instances, the vector insert is assembled via primary assembly PCR.

The term “secondary assembly PCR” refers to an assembly PCR reaction where initial double-stranded nucleic acid molecules are assembled to form product double-stranded nucleic acid molecules that are longer in length than the initial double-stranded nucleic acid molecules.

The term “primary amplification” refers to the first set of amplification reactions performed on the products of an assembly PCR reaction where single-stranded nucleic acid molecules are assembled to form double-stranded nucleic acid molecules. Later amplification cycles are termed “secondary”, “tertiary”, “quaternary”, etc. By way of illustration step 3 in FIG. 5A is a secondary amplification. Amplification cycles after a primary amplification may or may not result in amplification products that differ in length than starting nucleic acid molecules. The workflow distinguishes amplification cycles from each other. For example, FIG. 7 shows data resulting from primary amplification which occurs in the presence or absence of TkoEndoMS. Further, FIG. 7 shows data involving error correction using T7NI followed by secondary amplification.

### Nucleic Acid Molecule Generation

One of the first steps in producing a nucleic acid molecule or protein of interest, after the molecule(s) has been identified, is nucleic acid molecule design. A number of factors go into design of the nucleic acid sequence to be synthesized and the oligonucleotides used to generate the nucleic acid molecule. These factors include one or more of the following: (1) the AT/GC content of all or part of the nucleic acid molecule (e.g., the coding region), (2) the presence or absence of restriction endonuclease cleavage sites (including the addition and/or removal of restriction sites), (3) preferred codon usage for the particular protein production or host expression system that is to be employed, (4) junctions of the oligonucleotides being assembled, (5) the number and lengths of the oligonucleotides used to produce the desired nucleic acid molecule, (6) minimization of undesirable regions (e.g., “hairpin” sequences, regions of sequence homology to cellular nucleic acids, repetitive sequences, inhibitory cis-acting elements, restriction enzyme cleavage sites, internal splice sites etc.) and (7) coding region flanking segments that may be used for attachment of 5′ and 3′ components (e.g., restriction endonuclease sites, primer binding sites, sequencing adaptors or barcodes, recombination sites, etc.).

In many instances, parameters will be input into a computer and software will generate an in silico nucleotide sequence that balances the input parameters. The software may place “weights” on the input parameters in that, for example, what is considered to be a nucleic acid molecule that closely matches some of the input criteria may be difficult or impossible to assemble. Exemplary nucleic acid design methods are set out in U.S. Pat. No. 8,224,578. As further described below, the sequence design may also take into account requirements for multiplexing of oligonucleotides belonging to different subfragments of a product nucleic acid molecule.

Further, nucleic acid molecules design factors may be considered across the length of the nucleic acid molecule or in specific regions of the molecule. For example, GC content may be limited across the length of the nucleic acid molecule to prevent synthesis “failures” resulting from specific locations within the molecule. Thus, synthesizability of the nucleic acid molecule is a characteristic of the entire nucleic acid molecule in that a regional “failure to assemble” results in the designed nucleic acid molecule not being assembled. From a regional perspective, codon may be selected for optimal translation, but this may conflict with, for example, region limitation of GC content.

Assembly success often involves multiple parameters and regional characteristics of the desired nucleic acid molecule. Total and regional GC content is only one example of a parameter. For example, the total GC content of a nucleic acid molecule may be 50% but the GC content in a particular region of the same nucleic acid molecule may be 75%. Thus, in many instances, GC content will be “balanced” across the entire nucleic acid molecule and may vary regionally by less than 15%, 10%, 8%, 7%, or 5% from the total GC content.

The aim therefore is to reach a compromise which is as optimal as possible between satisfying the various requirements. In instances where the product nucleic acid molecule encodes a protein, the large number of amino acids in the protein leads to a combinatorial explosion of the number of possible DNA sequences which—in principle—are able to express the desired protein based on the degeneracy of the genetic code. For this reason, various computer-assisted methods have been proposed for ascertaining an optimal codon sequence.

Oligonucleotides or nucleic acid subfragments used for assembly PCR of a desired nucleic acid molecule may be derived from a number of sources, for example, they may be cloned, derived from polymerase chain reactions, chemically synthesized or purchased. In many instances, chemically synthesized nucleic acids tend to be of less than 100 nucleotides in length. PCR and cloning can be used to generate much longer nucleic acids. Further, the percentage of erroneous bases present in nucleic acids (e.g., nucleic acid fragments) is, to some extent, tied to the method by which it is made. Typically, chemically synthesized nucleic acids have the highest error rate.

A number of methods for chemical synthesis of oligonucleotides are known. In many instances, oligonucleotide synthesis is performed by a stepwise addition of nucleotides to the 5′-end of the growing chain until oligonucleotides of desired length and sequence are obtained. Further, each nucleotide addition can be referred to as a synthesis cycle and often consists of four chemical reactions: (1) De-Blocking/De-Protection, (2) Coupling, (3) Capping, and (4) Oxidation.

EGA and PGA deprotection reagents and methods for generating such acids, as well as their use in oligonucleotide synthesis are set out for example in Maurer et al., “Electrochemically Generated Acid and Its Containment to 100 Micron Reaction Areas for the Production of DNA Microarrays”, PLoS, Issue 1, e34 (2006), or in PCT Publications WO 2013/049227 and WO 2016/094512. Thus, in some instances, EGA is generated as part of the deprotection process. Further, in certain instances, all or part of the oligonucleotide synthesis reaction may be performed in aqueous solutions. In other instances, organic solvents will be used.

In many instances, a typical nucleic acid assembly PCR protocol may comprise a combination of methods set out herein, such as, for example, a combination of exonuclease-mediated generation of single-stranded overhangs followed by PCR-based assembly (referred to as a “standard workflow”). In some aspects, such standard workflow may comprise at least the following steps: (i) synthesizing single-stranded oligonucleotides together comprising a sequence of a desired assembly product, wherein each oligonucleotide has a sequence region that is complementary to a sequence region in another oligonucleotide, (ii) hybridizing the oligonucleotides via their complementary sequence regions and elongating the oligonucleotides in an overlap extension PCR reaction (primary assembly PCR) to assemble one or more double-stranded nucleic acid molecules, (iii) amplifying the assembled nucleic acid molecules in the presence of terminal primers (primary amplification), (iv) purifying the amplified nucleic acid molecules, (v) generating single-stranded overhangs at the terminal ends of the amplified one or more nucleic acid molecules and optionally, generating single-stranded overhangs at the terminal ends of a linearized target vector for subsequent cloning (e.g., by treatment of the fragments with one or more restriction endonucleases and/or an exonuclease), (vi) inserting the one or more nucleic acid molecules into the target vector via the complementary single-stranded overhangs, optionally followed by a ligation step, and (vii) transforming host cells (such as, e.g., E. coli) with the resulting vector construct. In some aspects, assembled nucleic acid molecules may be ligated “in vivo” by endogenous enzymatic activities of the transformed cell. For example, a gapped or nicked assembly product may be directly transformed into E. coli and gaps or nicks may be repaired by the E. coli endogenous repair machinery.

Two methods for assembling nucleic acid molecules are depicted in FIGS. 1A and 1B. These methods both involve starting with oligonucleotides or subfragments that will generally contain sequences that are overlapping at their termini which are “stitched” together via these complementary sequence regions using PCR. In some aspects, the overlaps are approximately 10 base pairs; in other aspects, the overlaps may be 15, 25, 30, 50, 60, 70, 80 or 100 base pairs, etc. (e.g., from about 10 to about 120, from about 15 to about 120, from about 20 to about 120, from about 25 to about 120, from about 30 to about 120, from about 40 to about 120, from about 10 to about 40, from about 15 to about 50, from about 40 to about 80, from about 60 to about 90, from about 20 to about 50, from about 15 to about 35, etc. base pairs). In order to avoid mis-assembly, individual overlaps will typically not be duplicated or closely matched amongst the subfragments. Since hybridization does not require 100% sequence identity between the participating nucleic acid molecules or regions, each terminus should be sufficiently different to prevent mis-assembly. Further, termini intended to undergo homologous recombination with each other should share at least 90%, 93%, 95%, or 98% sequence identity.

Further, multiple cycles of polymerase chain reactions may be used to generate successively larger nucleic acid molecules. In many instances, stitched oligonucleotides will be chemically synthesized and will be less than 100 nucleotides in length (e.g., from about 40 to 100, from about 50 to 100, from about 60 to 100, from about 40 to 90, from about 40 to 80, from about 40 to 75, from about 50 to 85, etc. nucleotides). Primers may also be used which contain restriction sites for instances where insertion into a cloning vector is desired. Where desirable, assembled nucleic acid molecules may be directly inserted into vectors and host cells. PCR-based insertion into a target vector may be appropriate when the desired construct is fairly small (e.g., less than 5 kilobases).

A standard workflow is represented in FIG. 1A by the basic steps of oligonucleotide synthesis, primary assembly PCR to assemble the oligonucleotides, primary amplification to amplify the assembled product, followed by purification of the amplified product, treatment with a nuclease to generate single-stranded overlaps between the purified insert and a target vector, and insertion of the insert into the target vector followed by a transformation step.

Another assembly PCR method comprises a combined sequence elongation and ligation reaction (FIG. 1B), wherein steps (ii), (iii) and (vi) of the standard workflow described above are combined in a single (“one-pot”) reaction, whereas other steps (such as steps (iv) and (v)) may be omitted. In particular, such methods comprise direct assembly (primary assembly PCR) of single-stranded overlapping oligonucleotides into a linearized target vector via overlap extension PCR and amplification (primary amplification) of the resulting subfragment-vector fusion construct in a single step. According to some aspects, no separate PCR reaction is required to generate double-stranded subfragments prior to vector insertion. Instead, the single-stranded oligonucleotides together representing at least a portion of a polynucleotide to be assembled can be directly used in the overlap extension reaction. After an initial denaturation step to separate the strands of a given linearized vector, the single-stranded oligonucleotides are annealed via their complementary ends. Two of the oligonucleotides are designed to carry sequence homologies with the vector backbone allowing for hybridization with the ends of one of the denatured vector strands. The 3′ends of the annealed oligonucleotides and/or the 3′ ends of the vector strands serve as primers for the synthesis of the complementary nucleic acid strand. The polymerase-mediated elongation stops when the 5′ end of a hybridized oligonucleotide is encountered resulting in the production of a nicked circularized double-stranded nucleic acid molecule. The fused and amplified assembly products can be directly transformed into host cells without further purification. In some aspects, no ligation step is performed prior to the transformation. The final ligation of the nicked fusion construct is achieved endogenously within the host cell.

In assembly chain reactions, overlapping oligonucleotides are assembled into linear double-stranded DNA fragments by successive cycles of denaturation, annealing and reciprocal extension of the oligonucleotides (primary assembly PCR) (see FIG. 2). In subsequent amplification reactions, nucleic acid molecules formed by assembly PCR can be amplified by PCR using terminal primers to generate and/or amplify assembled nucleic acid molecules (primary amplification), that may be used “as in” or in downstream processes (e.g., insertion into a vector, see FIG. 1A).

In some aspects set out herein, one or more thermostable mismatch recognition proteins are present in assembly PCR and/or amplification reactions (see, e.g., FIG. 2). The inclusion of thermostable mismatch recognition proteins allows for multiple rounds of error correction and/or error suppression to be performed with the need to add mismatch recognition proteins after denaturation steps. Thus, mismatch recognition proteins may be employed to decrease the number and/or percentage of nucleic acid molecules in a population that contains correct nucleic acid molecules and nucleic acid molecules which contain errors.

A schematic of one process for the correction of error in nucleic acid molecules during amplification (primers not shown) is set out in FIG. 3. This schematic shows single-stranded nucleic acid molecules in the upper left, some of which contain point mutations (indicated as ovals and circles). There is a high probability that, upon hybridization, the single-stranded nucleic acid molecules with the point mutations will hybridize with nucleic acid molecules that do not contain the same point mutation. The net result of this is a “mismatch”. The population of double-stranded nucleic acid molecules is then contacted with a mismatch endonuclease which cleaves nucleic acid molecules containing recognized mismatches, rending the cleave nucleic acid molecules unsuitable for logarithmic amplification. Of course, other methods may also be used to inhibit logarithmic amplification of nucleic acid molecules containing mismatches. For example, a mismatch binding protein may be used to either remove nucleic acid molecules containing mismatches or inhibit amplification of such nucleic acid molecules. Additionally, an error reducing polymerase reagent may be used during amplification.

In more detail, FIG. 3 shows a workflow of an exemplary process for synthesis of error-minimized nucleic acid molecules. In the first step, nucleic acid molecules of a length smaller than that of nucleic acid molecules assembled are obtained. Each of the smaller nucleic acid molecule is intended to have a desired nucleotide sequence that comprises a part of an assembled nucleic acid molecule. In the second to last step of the process set out in FIG. 3, annealed nucleic acid molecules are reacted with one or more exonucleases as part of the error correction process. Some variations of this process are as follows. First, two or more (e.g., two, three, four, five, six, etc.) rounds of error correction may be performed. Second, more than one endonuclease may be used in one or more rounds of error correction. For example, T7NI and Cel II may be used in each round of error correction. Third, different endonucleases may be used in different error correction rounds. For example, T7NI and Cel II may be used in a first round of error correction and TkoEndoMS may be used alone in a second round of error correction.

In many instances, a ligase may be present in reaction mixtures during error correction. It is believed that some endonucleases used in error correction processes have nickase activity. The inclusion of one or more ligase is believed to seal nicks caused by such enzymes and increase the yield of error corrected nucleic acid molecules after amplification. Exemplary ligases that may be used are T4 DNA ligase, Taq ligase, and PBCV-1 DNA ligase. Ligases used in the practice of methods et out herein may be thermolabile or thermostable (e.g., Taq ligase). If a thermolabile ligase is employed, it will typically need to be readied to a reaction mixture for each error correction round. Thermostable ligases will typically not need to be readded during each round, so long as the temperature is kept below their denaturation point.

In many instances, error correction of nucleic acid molecules may be mediated by one or more different mismatch recognition proteins. Examples of categories of such proteins are mismatch binding proteins and mismatch endonucleases. Further, mismatch binding proteins and mismatch endonucleases may be thermostable or non-thermostable, which will often depend on factors the conditions under which the proteins are used and biological activities of the specific protein (e.g., the type of errors recognized).

One exemplary method of error correction that may be used in methods described herein is set out in FIGS. 4 and 5A. FIG. 4 is a flow chart of an exemplary process for synthesis of error-minimized nucleic acid molecules. In the first step (line 1), nucleic acid molecules (e.g., oligonucleotides) of a length smaller than that of a nucleic acid molecule assembled therefrom are obtained. Each oligonucleotide is intended to have a desired nucleotide sequence that comprises a part of the nucleotide sequence of an assembled nucleic acid molecule. Each oligonucleotide may also be intended to have a nucleotide sequence that comprises one or more of the following: (1) An adaptor primer for PCR amplification of the nucleic acid molecule, a recognition site for a restriction enzyme, (2) a tethering sequence for attachment to a microchip or solid support, or (3) any other nucleotide sequence determined by any experimental purpose or other intention. The oligonucleotides may be obtained in any of one or more ways as described elsewhere herein, for example, through synthesis, purchase, etc.

In the optional second step (FIG. 4, line 2), the oligonucleotides are amplified to obtain more of each oligonucleotide. In many instances, however, sufficient numbers of oligonucleotides will be produced so that amplification is not necessary. When employed, amplification may be accomplished by any method known in the art, for example, by PCR, Rolling Circle Amplification (RCA), Loop Mediated Isothermal Amplification (LAMP), Nucleic Acid Sequence Based Amplification (NASBA), Strand Displacement Amplification (SDA), Ligase Chain Reaction (LCR), Self Sustained Sequence Replication (3SR) or solid phase PCR reactions (SP-PCR) such as Bridge PCR etc. (see, e.g., Fakruddin et al., J. Pharm. Bioallied. Sci. 5(4):245-252 (2013) for an overview of the various amplification techniques). Introduction of additional errors into the nucleotide sequences of any of the nucleic acid molecules may occur during amplification. In some instances, it may be favorable to avoid amplification following synthesis. The optional amplification step may be omitted where nucleic acid molecules have been produced at sufficient yield in step 1. This may be achieved by using, for example. optimized bead formats, designed to allow synthesis of nucleic acid molecules at sufficient yield and quality as described, for example, in PCT Publication WO 2016/094512.

In the third step (line 3 of FIG. 4), the optionally amplified nucleic acid molecules are assembled (primary assembly PCR) into a first set of nucleic acid molecules intended to have a desired length. Of course, in some instances, the nucleic acid molecule of line 3 may be a subfragment of an even larger nucleic acid molecule.

In the fourth step (line 4 of FIG. 4), the first set of assembled nucleic acid molecules is denatured. Denaturation renders single-stranded molecules from double-stranded molecules. Denaturation may be accomplished by any means. In some aspects, denaturation is accomplished by heating the molecules.

In the fifth step (line 5 of FIG. 4), the denatured molecules are annealed. Annealing renders a second set of double-stranded nucleic acid molecules from single-stranded molecules. Annealing may be accomplished by any means. In some aspects, annealing is accomplished by cooling the molecules. Some of the annealed molecules may contain one or more mismatches representing sites of sequence error.

In the sixth step (line 6 of FIG. 4), the second set of molecules are reacted with one or more mismatch cleaving endonucleases to yield a third set of nucleic acid molecules intended to have lengths less than the length of the complete desired gene sequence. Exemplary mismatch binding and/or cleaving enzymes are set out elsewhere herein but include T7NI, endonuclease VII (encoded by the T4 gene 49), RES I endonuclease, CEL I endonuclease, an EndoMS (e.g., PfuEndoMS, TkoEndoMS, etc.), and SP endonuclease or an endonuclease containing enzyme complex. These endonucleases generally function by cleaving (single-stranded of double-stranded cleaving) one or more of the molecules in the second set into shorter molecules. Cleavage at the sites of any nucleotide sequence errors are particularly desirable, in that assembly of pieces of one or more molecules that have been cut at error sites offers the possibility of removal of the cut errors in the final step of the process.

In the seventh step (line 7 of FIG. 4), the third set of molecules is assembled into a fourth set of molecules, whose length is intended to be the full-length of the desired nucleotide sequence. In the seventh step, which is typically based on overlap extension PCR, the 3′->5′ exonuclease activity of the DNA polymerase removes the 3′ overhangs generated by endonuclease cleavage in the sixth step at sites of mismatch thereby removing the error. Thus, the intrinsic exonuclease activity of a DNA polymerase can be used to remove errors during assembly that have not been removed in step 6 (e.g., by using a combination of nucleases with mismatch cleavage and exonuclease activities). This principle is outlined, e.g., in Saaem et al. (“Error correction of microchip synthesized genes using Surveyor nuclease”, Nucl, Acids Res., 40:e23 (2012)). Such final assembly step may be performed in the presence of terminal primers thereby including functionalities required for downstream processes such as cloning or protein expression. A respective PCR reaction may be set up to first allow the error-corrected fragments to assemble by overlap extension to the full-length in about 15 cycles of denaturation, annealing and extension in the absence of the terminal primers, followed by additional 20 cycles in the presence of the terminal primers.

The process set out above and in FIG. 4 is also set out in U.S. Pat. No. 7,704,690. Furthermore, the process described above may be encoded onto a computer-readable medium as processor-executable instructions.

One representative workflow that may be used in methods set out in FIG. 5A. In this workflow, three nucleic acid subfragments (line 1) are pooled and subjected to error correction using the enzyme T7 endonuclease I (“T7NI”) (line 2). The resulting products are then assembled by PCR (line 3) and then subjected to a second round of error correction (line 4). After another round of PCR (line 5), the resulting nucleic acid molecules are transformed into E. coli (step 6) and then screened for those that are full-length (line 7) followed by DNA preparation (line 8). These nucleic acid molecules may then be screened for remaining errors by, for example, sequencing (line 9). In a first variation of the workflow of FIG. 5A, the pooled subfragments may be treated with an exonuclease (such as, e.g., Exonuclease I) before they are subjected to the error correction process. Exonuclease treatment eliminates single-stranded primer molecules left over in the PCR reaction product that may interfere with subsequent PCR reactions and generate unspecific amplification products. In a second variation of the workflow, the first error correction step may use more than one endonuclease such as, for example, T7NI combined with RES I. Optionally, the workflow may comprise a third error correction or error removal step to eliminate remaining mismatches after fragment fusion PCR. Such third step may be conducted with a mismatch binding protein such as, for example, MutS. The skilled person will understand that various orders and combinations of first, second and/or third and possibly further error correction and/or removal rounds may be applied to further decrease the error rate of assembled nucleic acid molecules.

Another process for effectuating error correction in chemically synthesized nucleic acid molecules that may be used in methods set out herein is by a commercial process referred to as ERRASE™ (Novici Biotech).

A variation of the workflow of FIG. 5A is outlined in FIG. 5B. In this embodiment, three subfragments (FIG. 5B, Line 1) are pooled and treated with an exonuclease (such as, e.g., Exonuclease I; Line 2a in the workflow on the right) before being subjected to the double error correction processing (FIG. 5B, Lines 2b and 4). The exonuclease eliminates single stranded primer molecules left over in the PCR reaction product that may interfere with subsequent PCR reactions (Line 3) and generate unspecific amplification products. In another variation of the workflow, the first error correction step may use more than one endonuclease such as, e.g., T7NI combined with RES I (FIG. 5B, Line 2b). Optionally, the workflow may comprise a third error correction step to eliminate remaining mismatches after segment assembly PCR (Line 3, secondary assembly PCR in this instance 3). Such third error correction step may be conducted with a mismatch binding protein such as, e.g., MutS (Line 4). Of course, various orders and combinations of first, second and/or third and possibly further error correction rounds may be applied to further decrease the error rate of assembled nucleic acid molecules.

Using the workflow set out in FIG. 5A for purposes of illustration, nucleic acid molecules containing errors may be removed at one or more steps. For example, “mismatched” nucleic acid molecules may be removed between steps 1 and 2 and/or before step 1 in FIG. 5A. This would result in the treatment of a “preselected” population of nucleic acid molecules with a mismatch endonuclease. Further, two error correction steps such as these may be used in combination. As an example, nucleic acid molecules may be denatured, then reannealed, followed by removal of nucleic acid molecules with mismatches through binding with immobilized MutS, then followed by contacting the nucleic acid molecules that are not separated by MutS binding with a mismatch endonuclease without intervening denaturation and reannealing steps. While not wishing to be bound by theory, it is believed that amplification of nucleic acid molecules introduces errors into the molecules being amplified. One means of avoiding the introduction of amplification mediated errors and/or for the removal of such errors is by the selection of nucleic acid molecules with correct sequences after most or all amplification steps have been performed. Again using the workflow set out in FIG. 5B for purposes of illustration, nucleic acid molecules with mismatches may be separated from those without mismatches after step 5 by an additional separation step using a mismatch binding protein (not shown in FIG. 5B).

Variations of this process are as follows. First, two or more (e.g., two, three, four, five, six, etc.) rounds of error correction may be performed, and in each round a thermostable mismatch recognition protein may be used. Second, more than one endonuclease may be used in one or more rounds of error correction. For example, T7NI and Cel II may be used in each round of error correction. Third, different endonucleases may be used in different error correction rounds or may be combined with steps of error filtration using mismatch binding proteins. For example, a pool of re-annealed oligonucleotides may be subject to an error filtration step using a mismatch binding protein (such as MutS) to remove a first plurality of oligonucleotides having errors from the pool (see FIG. 5B) and the remaining (“unbound”) oligonucleotides may then be subject to an error correction step using an endonuclease such as, e.g., T7NI to correct remaining errors.

In some instances, T7NI and Cel II, for example, may be used in a first round of error correction and Cel II may be used alone in a second round of error correction. Of course, other mismatch endonucleases may also be used. In another exemplary embodiment, the molecules are cleaved only with one endonuclease (which may be a single-strand nuclease, such as Mung Bean endonuclease or a resolvase, such as T7NI or another endonuclease of similar functionality). In yet another embodiment the same endonuclease (e.g., T7NI) may be used in two subsequent error correction rounds (line 4 of FIG. 5A). In yet another embodiment an enzyme having mismatch cleavage activity may be combined with an enzyme having exonuclease activity to allow for removal of errors contained in single-stranded overhangs following mismatch cleavage. In specific aspects, mismatch endonucleases having intrinsic exonuclease activity may be used to achieve cleavage and subsequent error removal in a single step. Enzymes having both endonuclease and exonuclease activities include, for example, Mung Bean nuclease, Cel I or SP1 endonuclease. In other aspects, removal of errors may be achieved by a separate step comprising further exonuclease treatment as described, for example, in PCT Publication WO 2005/095605 A1.

In many instances, one or more ligase may be present in reaction during error correction. It is believed that some endonucleases used in error correction processes have nickase activity. The inclusion of one or more ligase is believed to seal nicks caused by such enzymes and increase the yield of error corrected nucleic acid molecules after amplification. Exemplary ligases that may be used are T4 DNA ligase, Taq ligase, and PBCV-1 DNA ligase. Ligases used in the practice of methods set out herein may be thermolabile or thermostable (e.g., Taq ligase). If a thermolabile ligase is employed, it will typically need to be added to a reaction mixture for each error correction round. Thermostable ligases will typically not need to be re-added during each round, so long as the temperature is kept below their denaturation point.

In instances where the second set of molecules represents a subfragment of a larger nucleic acid molecule, two or more subfragments (e.g., two or three or more subfragments) together representing the larger nucleic acid molecule may be combined and reacted with the one or more mismatch cleaving endonucleases in a single reaction mix. For example, where the open reading frame that is to be assembled is longer than 1 kb, it may be broken up into two or more subfragments separately assembled in parallel reactions in step three and the resulting two or more subfragments may be combined and error-corrected in a single reaction as indicated in FIG. 5A. The amount of subfragments to be combined in a single error correction round may depend on the length of the individual subfragments. For example, up to three subfragments of about 1 kb in length may be efficiently combined in a single reaction mixture. Of course, more than three (e.g., four, five, six, seven, eight, nine, etc.) subfragments may be combined. Assembly efficiency may decrease so long as at least one correctly assembled amplifiable and/or replicable nucleic acid molecule is obtained. Thus, numerous subfragments (e.g., subfragments of about 1 kb in length) may be assembled so long as a correctly assembled product nucleic acid molecule is obtained from the assembly process.

Nucleic acid molecules with mismatches may be separated from those without mismatches by binding with a mismatch binding agent in a number of ways. For example, mixtures of nucleic acid molecules, some having mismatches, may be (1) passed through a column containing a bound mismatch binding protein or (2) contacted with a surface (e.g., a bead (such as a magnetic bead), plate surface, etc.) to which a mismatch binding protein is bound.

Exemplary formats and associated methods involve those using beads, or other supports, to which a mismatch binding protein is bound. For example, a solution of nucleic acid molecules may be contacted with beads to which is bound a mismatch binding protein. Nucleic acid molecules that are bound to the mismatch binding protein are then linked to the surface and not easily removed or transferred from the solution.

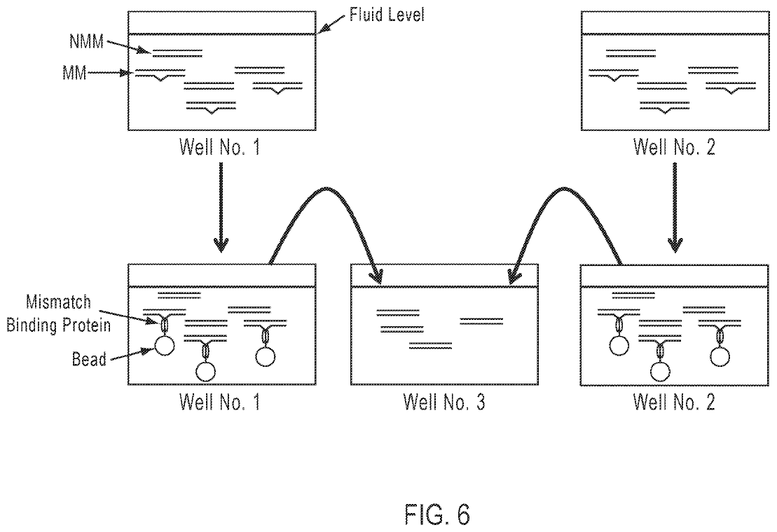

In a specific format set out in FIG. 6, beads with a bound mismatch binding protein may be placed in a vessel (e.g., a well of a multi-well plate) with nucleic acid molecules present in solution, under conditions that allow for the binding of nucleic acid molecules with mismatches to the mismatch binding protein (e.g., 5 mM MgCl₂, 100 mM KCl, 20 mM Tris-HCl (pH 7.6), 1 mM DTT, 25° C. for 10 minutes). Fluid may then be transferred to another vessel (e.g., a well of a multi-well plate) without transferring of the beads and/or mismatched nucleic acid molecules. One specific type of bead that can be used are Magnetic Mismatch Binding Beads (M2B2), MAGDETECT™ (United States Biological, Salem, MA, cat. no. M9557-01A). Further, mismatch binding protein used in workflows similar or identical to that set out in FIG. 6 may be thermostable or non-thermostable.

As an example, a protein that has been shown to bind double-stranded nucleic acid molecules containing mismatches is E. coli MutS (Wagner et al., Nucleic Acids Res., 23:3944-3948 (1995)). Wan et al., Nucleic Acids Res., 42:e102 (2014) demonstrated that chemically synthesized nucleic acid molecules containing errors can be retained on a MutS-immobilized cellulose column with nucleic acid molecules not containing errors not being so retained.

Subject matter set out herein thus includes methods, as well as associated compositions, in which nucleic acid molecules are denatured, followed by reannealing, followed by the separation of reannealed nucleic acid molecules containing mismatches. In some aspects, the mismatch binding protein used is MutS (e.g., E. coli MutS). Of course, other mismatch binding proteins, such as those set out in Tables 12 and 15, may also be used.

Further, mixtures of mismatch binding proteins may be used in the practice of methods set out herein. It has been found that different mismatch binding proteins have different activities with respect to the types of mismatches they bind to. For example, Thermus aquaticus MutS has been shown to effectively remove insertion/deletion errors but is less effective in removing substitution errors than E. coli MutS. Further, a combination the two MutS homologs was shown to further improve the efficiency of the error correction with respect to the removal of both substitution and insertion/deletion errors, and also reduced the influence of biased binding. Subject matter set out herein thus includes mixtures of two or more (e.g., from about two to about ten, from about three to about ten, from about four to about ten, from about two to about five, from about three to about five, from about four to about six, from about three to about seven, etc.) mismatch binding proteins.

Subject matter set out herein further includes the use of multiple rounds (e.g., from about two to about ten, from about three to about ten, from about four to about ten, from about two to about five, from about three to about five, from about four to about six, from about three to about seven, etc.) of error correction using mismatch binding proteins. One or more of these rounds of error correction may employ the use of two or more mismatch binding proteins. Alternatively, a single mismatch binding protein may be used in a first round of error correction whereas the same or another mismatch binding protein may be used in a second round of error correction.

Once the oligonucleotide synthesis has been completed, the resulting oligonucleotides are typically subjected to a series of post processing steps that may include one or more of the following: (a) cleavage of the oligonucleotides or elution from the support upon which they were synthesized, (b) concentration measurement, (c) concentration adjustment or dilution of oligonucleotide solutions, often referred to as “normalization”, to obtain equally concentrated dilutions of each oligonucleotide species, and/or (d) pooling or mixing aliquots of two or more normalized oligonucleotide samples to obtain equimolar mixtures of all oligonucleotides required to assemble one or more specific nucleic acid molecules, wherein the aforementioned steps may be combined in different orders.

Yet another process for reducing errors during nucleic acid synthesis that may be used in aspects of subject matter set out herein is referred to as Circular Assembly Amplification and described in PCT Publication WO 2008/112683 A2.

Synthetically generated nucleic acid molecules typically have error rate of about 1 base in 300-500 bases. Conditions can be adjusted so that synthesis errors are substantially lower than 1 base in 300-500 bases. Further, in many instances, greater than 80% of errors are single base frame shift deletions and insertions. Also, less than 2% of errors result from the action of polymerases when high fidelity PCR amplification is employed. Therefore, error-correction processes using PCR-based assembly steps as described above may be combined with one or more error-correction methods not involving polymerase activity. In many instances, mismatch endonuclease (MME) correction will be performed using fixed protein:DNA ratio. Non-PCR-based error correction may, e.g., be achieved by separating nucleic acid molecules with mismatches from those without mismatches by binding with a mismatch binding agent in a number of ways. For example, mixtures of nucleic acid molecules, some having mismatches, may be (1) passed through a column containing a bound mismatch binding protein or (2) contacted with a surface (e.g., a bead (such as a magnetic bead), plate surface, etc.) to which a mismatch binding protein is bound.

Exemplary formats and associated methods involve those using surfaces or supports (e.g., beads) to which a mismatch binding protein is bound. For example, a solution of nucleic acid molecules may be contacted with beads to which is bound a mismatch binding protein. One mismatch binding protein that may be used in various aspects of methods set out herein is MutS from Thermus aquaticus the gene sequence of which is published in Biswas and Hsieh, J. Biol. Chem. 271:5040-5048 (1996) and is available in GenBank, accession number U33117. Furthermore, mismatch cleavage endonucleases such as an EndoMS (e.g., PfuEndoMS, TkoEndoMS, etc.), T7NI or Cel I from, for example, celery may be genetically engineered to inactivate the cleavage function for use in error filtration processes based on mismatch binding. Nucleic acid molecules that are bound to a mismatch binding protein may either be actively removed from a pool of nucleic acid molecules (e.g., via magnetic force where magnetic beads coated with mismatch binding proteins are used) or may be immobilized or linked to a surface such that they remain in the sample whereas unbound nucleic acids are removed or transferred (e.g., by pipetting, acoustic liquid handling etc.) from the sample. Such methods are set out, for example, in PCT Publication WO 2016/094512.

As indicated above, mismatch recognition proteins may be used in conjunction with the hybridization of nucleic acid molecules. Mismatch recognition proteins included in compositions and used in methods set out herein may be thermostable or non-thermostable. Further, methods set out herein include those where more than one mismatch recognition protein is used at more than one location in nucleic acid related workflows (e.g., assembly PCR, amplification, error correction alone, or one or more combinations of these processes).

Thermostable mismatch recognition proteins (e.g., one or more thermostable mismatch endonuclease) allow for the elimination of sequence errors during processes such as assembly PCR, amplification and error correction without the need for re-addition of mismatch recognition protein after each thermal denaturation step. Thus, compositions and methods set out herein allow for the multiple rounds of error correction where mismatch recognition protein is not added after each nucleic acid denaturation step. Of course, non-thermostable mismatch recognition proteins may also be used in such workflows but mismatch recognition activity of such proteins would generally be eliminated or substantially decreased by each thermal denaturation cycle. In many instances, it would be necessary or desirable to add more non-thermostable mismatch recognition proteins after each thermal denaturation cycle.

The type or types of mismatch recognition proteins used in workflows may vary. In some instances, error correction may be performed at one or more location in a workflow. In some instances, a thermostable mismatch recognition protein will be used and, often, in conjunction with a non-thermostable mismatch recognition protein.

One method for removing nucleic acid molecules with errors is by the separation of such nucleic acid molecules from nucleic acid molecules that do not contain errors. Thus, provided herein are workflows, and composition used in such workflows, that use agents that bind to nucleic acid molecules containing errors and the separation of them from nucleic acid molecules that do not contain errors. Examples of such agents are mismatch binding proteins.

Mismatch binding proteins may be bound to a support, for example, may be contacted with a sample containing nucleic acid molecules with mismatches and nucleic acid molecules without mismatches under conditions where the nucleic acid molecule with mismatches will be bound to the support. The support to which nucleic acid molecule with mismatches are bound may then be removed from contact with nucleic acid molecules without mismatches, thereby separating nucleic acid molecules with mismatches from nucleic acid molecules without mismatches.

Another method for increasing the percentage of correct nucleic acid molecules in a composition is by suppressing amplification of nucleic acid molecules containing errors (e.g., deletions, insertion, mismatches, etc.). In some instances, one or more protein (e.g., one or more mismatch binding proteins) may be used which reduces the number of errors in a population of nucleic acids molecules by inhibiting assembly PCR and/or amplification of nucleic acid molecules that contain one or more error. In some instances, a polymerase reagent may be used which reduces the number of errors in a population of nucleic acids molecules by disfavoring assembly PCR and/or amplification of nucleic acid molecules that contain one or more error.

Some examples of workflows that may be performed are set out in Table 1.

TABLE 1
Exemplary Error Correction Workflows Based Upon Workflow of FIG. 1A.

| | Error Correction Reagents | | | |
|---|---|---|---|---|
| | | | Secondary | |
| No. | Primary Assembly PCR | Primary Amplification | Amplification | ERP |
| 1 | TsMME | No reagent | NTsMME | No |
| 2 | TsMME | TsMME | NTsMME | No |
| 3 | TsMME | TsMME | NTsMME | Yes |
| 4 | TsMME and TsMBP | TsMME | NTsMME | Yes |
| 5 | TsMME and TsMBP | No Reagent | NTsMME | No |
| 6 | TsMBP | No reagent | NTsMME | No |
| 7 | TsMBP | TsMME | NTsMME | Yes |
| 8 | TsMME | TsMME | TsMME | Yes |
| 9 | Two different TsMMEs | TsMME | Two different | No |
| | | | NTsMMEs | |
| 10 | Several different TsMMEs and | Several different TsMMEs and | NTsMME | Yes |
| | several different TsMBPs | several different TsMBPs | | |
| 11 | Several different TsMMEs and | Several different TsMMEs and | Two different | Yes |
| | several different TsMBPs | several different TsMBPs | NTsMMEs | |
| 12 | Several different TsMMEs and | Several different TsMMEs and | No treatment | No |
| | several different TsMBPs | several different TsMBPs | | |
| 13 | Several different TsMMEs and | Several different TsMMEs and | No treatment | Yes |
| | several different TsMBPs | several different TsMBPs | | |

ERP: Error Reducing Polymerase Reagent
TsMME: Thermostable Mismatch Endonuclease
NTsMME: Non-Thermostable Mismatch Endonuclease
TsMBP: Thermostable Mismatch Binding Protein
NTsMBP: Non-Thermostable Mismatch Binding Protein
Note:
Secondary amplification is not shown in FIG. 1A.

As shown by, for example, the workflow variations set out in Table 1, provided herein are compositions and methods for generating populations of nucleic acid molecules. In some such methods, these workflows comprise two or more different types of processes (e.g., nucleic acid assembly, nucleic acid amplification, nucleic acid denaturation/renaturation, etc.) in which single-stranded nucleic acid molecules hybridize to each other to form double-stranded nucleic acid molecules. In all or part of such workflows, either error correction or error reduction may occur. In some instances, error correct may occur between steps referenced in Table 1. For example, when one or more non-thermostable mismatch endonuclease (e.g., T7NI) is used after primary amplification, it will typically be contacted with amplification products before secondary amplification. This is so because thermal cycling will normally denature non-thermostable mismatch endonucleases. Mismatch binding proteins may also be used between amplifications steps where the mismatch binding proteins are used to separate mismatched nucleic acid molecule from non-mismatched nucleic acid molecules.

In some instances, the collective effect of processes set out herein may result in populations of nucleic acid molecules which contain fewer errors than 1 per 500 base pairs (e.g., from about 1 per 500 to about 1 per 2,000, from about 1 per 600 to about 1 per 2,000, from about 1 per 700 to about 1 per 2,000, from about 1 per 800 to about 1 per 2,000, from about 1 per 900 to about 1 per 2,000, from about 1 per 1,000 to about 1 per 2,000, from about 1 per 700 to about 1 per 1,500, from about 1 per 700 to about 1 per 1,200, from about 1 per 700 to about 1 per 1,000, from about 1 per 800 to about 1 per 1,200, etc. base pairs).

Addition of one or more mismatch binding protein (e.g., thermostable mismatch binding proteins) to assembly PCR mixtures may be used for functional removal of oligonucleotides containing sequence errors by blocking the extension by a polymerase when a mismatch binding protein is bound to the mismatch formed during annealing (see Fukui et al., “Simultaneous Use of MutS and RecA for Suppression of Nonspecific Amplification during PCR” J. Nucleic Acids, Volume 2013, Article ID 823730).

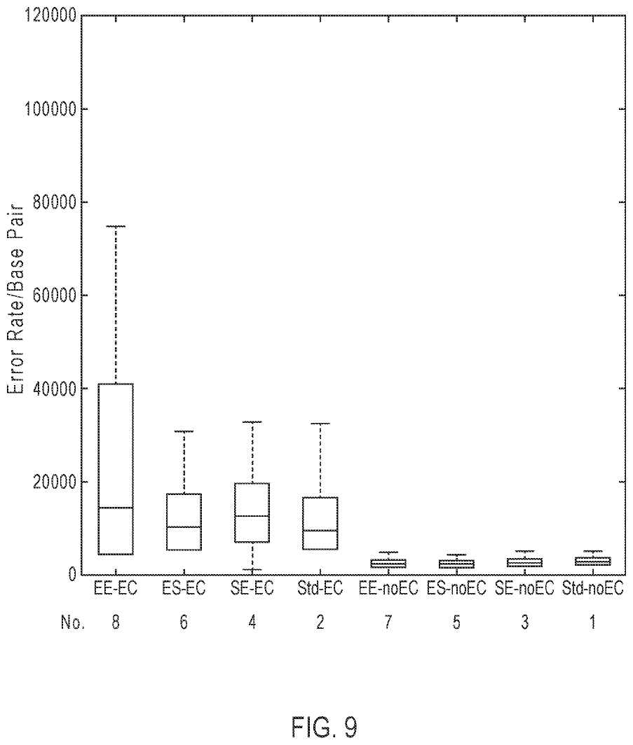

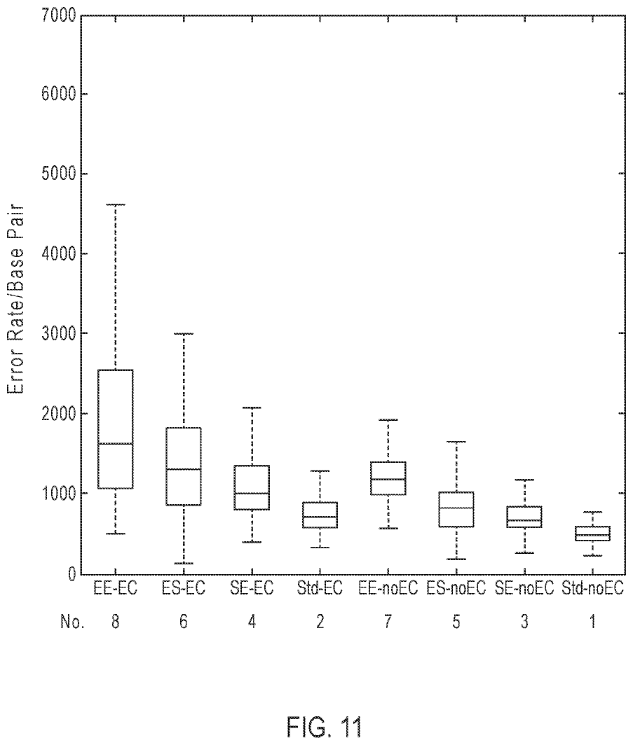

Mismatch-binding proteins and mismatch endonucleases often show specificity for certain types of mismatches. Thus, in some instances more than one mismatch recognition protein may be used in workflows set out herein. Further, in many instances, when more than one mismatch recognition protein is present, the error recognition activities of the proteins will differ. For example, the mismatch endonucleases TkoEndoMS and T7NI differ in that T7NI is believed to have higher activities with respect to deletions and insertions than TkoEndoMS (see FIGS. 9-11). Additionally, when more than one mismatch recognition protein is used, these proteins may have different activities with respect to different types of mismatches.

FIG. 7 shows data in which oligonucleotides were assembled by primary assembly PCR. The assembled nucleic acid molecules were then either subjected to primary amplification in the presence of TkoEndoMS and secondary amplification after incubation of the primary amplification product with or without T7NI. Resulting nucleic acid molecules were then sequenced to determine error rates.

Sample Number 1 (Std-noEC) was a control run where 66 fragments were assembled with no error correction. As can be seen from this figure, the median error rate for Sample Number 1 is 1 in 308. This increases to 1 in 456 when post-primary amplification T7NI mediated error correction was used (Sample Number 2). Sample Numbers 1 and 2 represents an error correction baseline of conditions in which there was no error correction and error correction using T7NI post-primary amplification of the assembled fragments.

The data for Sample Numbers 3 and 4 in FIG. 7 were generated under conditions where a thermostable mismatch endonuclease (TkoEndoMS) was present only in the amplification process but not in the assembly PCR process. Further, for Sample Number 4, post-primary amplification T7NI mediated error correction was used and for Sample Number 3 post-primary amplification T7NI mediated error correction was not used. As can be seen from FIG. 7, the error rate for Sample Number 3 is 1 in 353. This increases to 1 in 716 when post-primary amplification T7NI mediated error correction was used (Sample Number 4).

The data for Sample Numbers 5 and 6 in FIG. 7 were generated under conditions where a thermostable mismatch endonuclease (TkoEndoMS) was present in the assembly PCR process but not in the amplification process. Further, for Sample Number 6, post-primary amplification T7NI mediated error correction was used and for Sample Number 5 post-primary amplification T7NI mediated error correction was not used. As can be seen from FIG. 7, the median error rate for Sample Number 5 is 1 in 398. This increases to 1 in 830 when post-primary amplification T7NI mediated error correction was used (Sample Number 6).

The data for Sample Numbers 7 and 8 in FIG. 4 were generated under conditions where a thermostable mismatch endonuclease (TkoEndoMS) was present in both the assembly PCR and the amplification processes. Further, for Sample Number 8, post-primary amplification T7NI mediated error correction was used and for Sample Number 7 post-primary amplification T7NI mediated error correction was not used. As can be seen from FIG. 7, the median error rate for Sample Number 7 is 1 in 488. This increases to 1 in 803 when post-primary amplification T7NI mediated error correction was used (Sample Number 8).

The data set out in FIG. 7 shows that assembled and amplified nucleic acid molecules prepared using a thermostable mismatch endonuclease and subjected to T7NI mediated error correction have the lowest total error rate.

Table 1 below shows data derived from FIG. 7. It can be seen from Table 2 that the lowest levels of total errors present in nucleic acid molecules prepared using TkoEndoMS methods set out in below Example 1 were found in Sample Numbers 4, 6, and 8. These samples share the commonality that TkoEndoMS was present during (1) the assembly PCR process, (2) the amplification process, or (3) both the assembly PCR and the amplification processes. Further, all three of these samples were also subjected to post-primary amplification T7NI mediated error correction.

TABLE 2
FIG. 7 Data Fold Differences

Fold Difference

| Sample | Benchmark | Sample |
|---|---|---|
| No. | Avg. | Benchmark |
| 1 | 0.97 | 0.97 |
| 2 | 1.44 | 1.54 |
| 3 | 1.11 | 1.16 |
| 4 | 2.26 | 2.09 |
| 5 | 1.26 | 1.31 |
| 6 | 2.62 | 2.58 |
| 7 | 1.54 | 1.40 |
| 8 | 2.53 | 2.69 |

The data in FIG. 7 and Table 2 suggest that (1) the presence of a mismatch endonuclease in the assembly PCR process alone results in lower error rates than the presence of a mismatch endonuclease in the amplification process alone and (2) the inclusion of post-primary amplification mismatch endonuclease mediated error correction step provides error correction enhancement when used in combination with the use of thermostable mismatch endonuclease activity in the assembly PCR process and/or amplification process.

Provided herein are compositions and methods in which the error rates of assembled and amplified nucleic acid molecules is from about 1 in 500 to about 1 in 5,000 base pairs (e.g., from about 1 in 550 to about 1 in 1,500, from about 1 in 600 to about 1 in 1,500, from about 1 in 650 to about 1 in 1,500, from about 1 in 700 to about 1 in 1,500, from about 1 in 800 to about 1 in 1,500, from about 1 in 500 to about 1 in 1,400, from about 1 in 500 to about 1 in 1,350, from about 1 in 500 to about 1 in 1,300, from about 1 in 500 to about 1 in 1,250, from about 1 in 500 to about 1 in 1,200, from about 1 in 500 to about 1 in 1,150, from about 1 in 500 to about 1 in 1,000, from about 1 in 600 to about 1 in 1,000, from about 1 in 650 to about 1 in 1,000, from about 1 in 600 to about 1 in 900, from about 1 in 650 to about 1 in 900, from about 1 in 700 to about 1 in 850, from about 1 in 550 to about 1 in 2,000, from about 1 in 550 to about 1 in 2,500, from about 1 in 550 to about 1 in 3,500, from about 1 in 550 to about 1 in 4,500, from about 1 in 900 to about 1 in 3,500, from about 1 in 1,500 to about 1 in 5,000, from about 1 in 2,000 to about 1 in 5,000, from about 1 in 2,500 to about 1 in 5,000, etc. base pairs). Such nucleic acid molecule may be generated by primary assembly PCR and primary assembly, optionally followed by secondary amplification.

Provided herein are compositions and methods in which the fold decrease (“X”) in the error rate of assembled and amplified nucleic acid molecules is greater than 1.75 (e.g., from about 1.75 to about 8, from about 1.75 to about 7, from about 1.75 to about 8, from about 1.75 to about 5, from about 1.75 to about 4, from about 1.75 to about 3, from about 2.0 to about 8, from about 2.1 to about 8, from about 2.2 to about 8, from about 2.3 to about 8, from about 2.5 to about 8, from about 2.75 to about 8, from about 2.0 to about 7, from about 2.0 to about 6, from about 2.0 to about 5, from about 2.0 to about 4.5, from about 2.2 to about 8, from about 2.2 to about 7, from about 2.2 to about 6, from about 2.2 to about 5, from about 2.2 to about 3, from about 2.2 to about 2.8, from about 2.1 to about 2.8, etc.) when compared to the error rate of assembled and amplified nucleic acid molecules without error correction using either a single control/“benchmark” sample run or an average of control/“benchmark” sample runs (see data in FIG. 7 and Table 2). A formula that may be used to calculate the fold decrease in error rate is as follows:

$$X = \frac{Y}{Z}$$

where X is the fold decrease in errors, Y is the number of error rate after the error correction step, and Z is the number of error rate before the error correction step. FIG. 7, line 8 shows an error rate (Y) of 1 in 803. FIG. 7, line 1 shows an error rate (Y) of 1 in 308. Using these numbers, the fold decrease (X) in the error rate is 2.6.

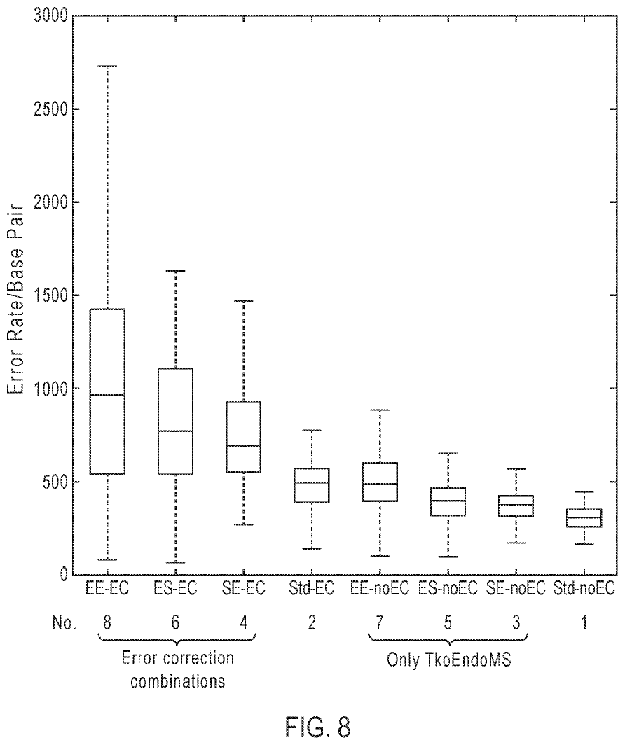

FIGS. 9, 10 and 11 show detailed data related to the error rates related to deletions, insertions and substitutions using the experimental data used to generate FIGS. 7 and 8.

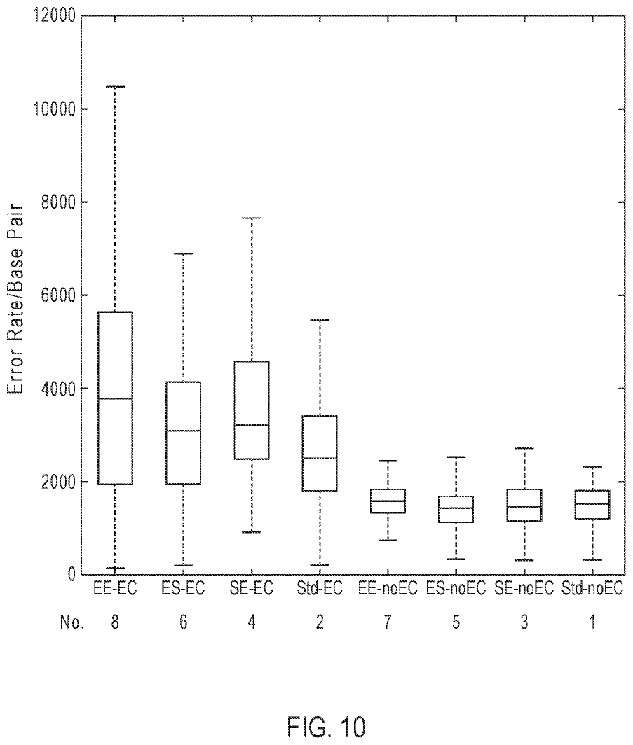

Sample Numbers 8, 6, 4, and 2 (T7NI treated) all show similarly low levels of deletions and insertions in FIGS. 9 and 10. These data indicate that deletions and insertions not removed by TkoEndoMS during assembly PCR and amplification are removed by post-primary amplification T7NI mediated error correction.

FIG. 10 shows that TkoEndoMS eliminates substitution errors when included in the assembly PCR process, the amplification process or both the assembly PCR and the amplification processes.

A number of different types of substitutions can be found in double-stranded nucleic acid molecules. Further, mismatch recognition proteins often vary in specificity of the types of substitutions they demonstrate activity towards. This specificity can vary with specific conditions, such as the presence or absence of divalent metal ions and the surrounding nucleic acid region. Some of these variations of EndoMS are set out in Ishino et al., Nucl. Acids Res. 44:2977-2989 (2016). Additional EndoMS proteins are set out in Table 15. Also, altered forms of wild-type thermostable mismatch endonuclease from Pyrococcus furiosus have been generated (see U.S. Pat. No. 10,196,618 and U.S. Patent Publication No. 2017/253909). Further, altered forms of wild-type mismatch recognition proteins (e.g., mismatch endonucleases) may be generated that vary in mismatch recognition activities. Such altered forms of wild-type mismatch recognition proteins may be included in and/or used in methods set out herein.

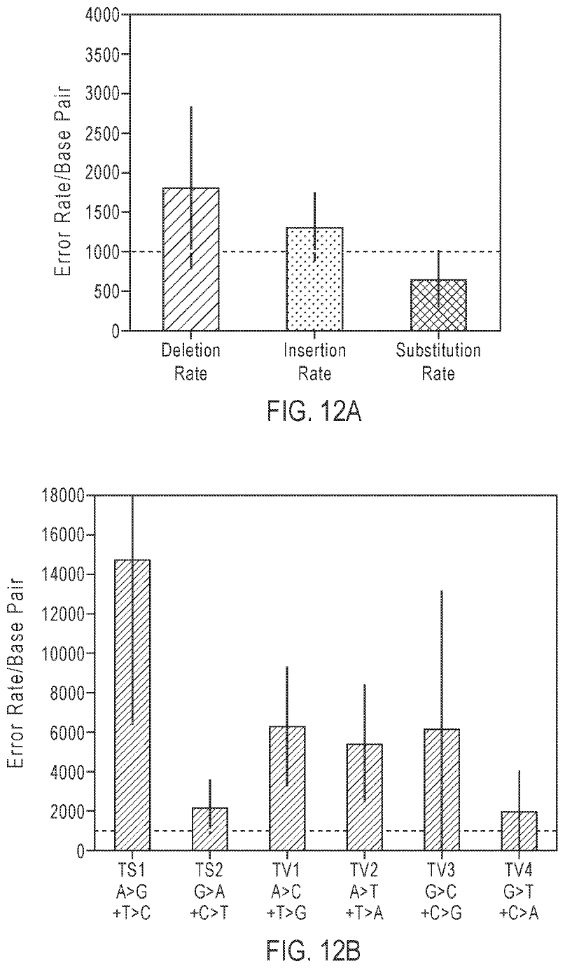

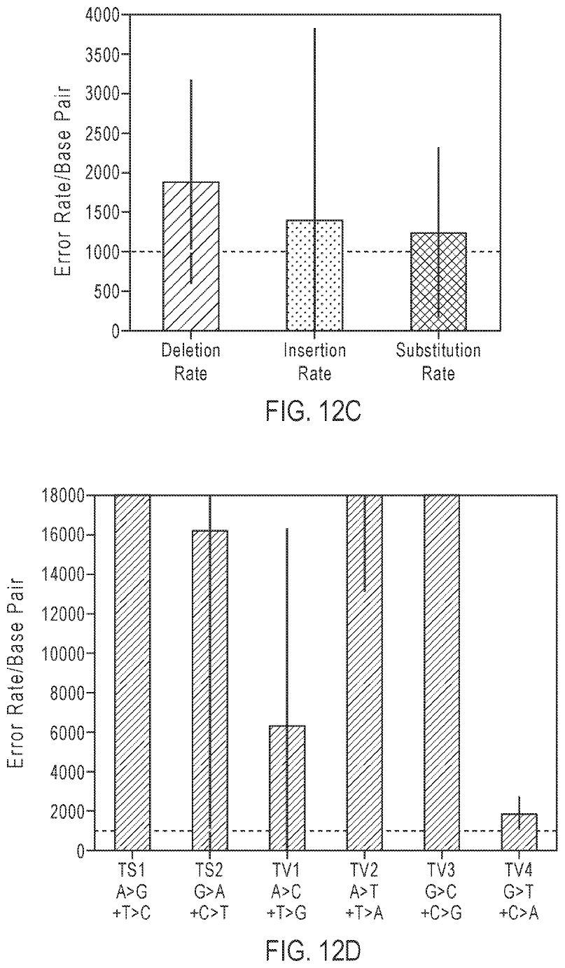

FIGS. 12A to 12D show some error correction properties of TkoEndoMS under the conditions used in Example 1. FIGS. 12A and 12C compare deletion, insertion and substitution levels found in assembled and amplified nucleic acid molecules generated in the absence of error correction (FIG. 12A) and where TkoEndoMS was included in both the assembly PCR process and amplification process (FIG. 12C). As can be seen, the number of deletions and insertions are similar under both sets of conditions. While there is significant variation in the data, it appears from these data that the substitution rate is lower when TkoEndoMS is present.

FIGS. 12B and 12D show some error correction activities of TkoEndoMS with respect to specific substitutions. While TkoEndoMS appears to be effective at correcting most transitions and transversions, it appears to have low activity related towards TV1 (C-T and G-A) and TV4 (C-T and G-A) mismatches (FIG. 12D). Further, T7NI also appears to have low activity related towards TV1 (C-T and G-A) and TV4 (C-T and G-A) mismatches (FIG. 12B).

SURVEYOR® nuclease is believed to cleave all types of mismatches but some are more preferred than others. In particular, C-T, A-C, and C-C are preferred equally over T-T, followed by A-A and G-G, and finally followed by the least preferred, A-G and G-T.

A number of mismatch recognition proteins (e.g., mismatch recognition proteins set out in Table 15) are known to have recognition activity for different types of mismatches. Error correction specificities of some mismatch recognition proteins are set out in Table 3.

TABLE 3
Activities of Selected Mismatch Recognition Proteins

| Gene/ | | | |
|---|---|---|---|
| Protein | Organism | Activity | Error Correction Specificity |
| PfuEndoMS | Pyrococcus | Cleavage | Branched DNA structures, |
| | furiosus | | G/T |
| TkoEndoMS | Thermococcus | Cleavage | G/T, G/G, T/T, T/C, A/G |
| | kodakarensis | | |
| TaqMutS | Thermus | Binding | InDels (1-4 bases), G/T, |
| | aquaticus | | T/C, A/G |
| TthMutS | Thermus | Binding | InDels (1-4 bases), G/T, |
| | thermophilus | | T/C, A/G |

Methods set out herein include those where more than one mismatch recognition protein are used in conjunction. Using the workflow shown in FIG. 1A for purposes of illustration, PfuEndoMS and TkoEndoMS can be used together in the oligonucleotide assembly process. This results in the presence of two different mismatch endonucleases that have overlapping but different error recognition activities. Further, one or both of TaqMutS and TthMutS may be used with each other or in conjunction with, for example, PfuEndoMS and TkoEndoMS for the elimination of double-stranded nucleic acid molecules that contain error recognized by them.

Provided herein are methods for the correction of error in nucleic acid molecules involving the sequence or simultaneous use of mismatch recognition proteins that differ in the types of errors they recognize,

Error correction methods and reagents suitable for use in methods provided herein are set out in U.S. Pat. Nos. 7,838,210 and 7,833,759, U.S. Patent Publication No. 2008/0145913 A1 (mismatch endonucleases), PCT Publication WO 2011/102802 A1, and in Ma et al., Trends in Biotechnology, 30(3):147-154 (2012). Furthermore, the skilled person will recognize that other methods of error correction and/or error filtration (i.e., specifically removing error-containing molecules) may be practiced in certain aspects of subject matter set out herein such as those described, for example, in U.S. Patent Publication Nos. 2006/0127920 AA, 2007/0231805 AA, 2010/0216648 A1, or 2011/0124049 A1.

Provided herein are compositions and methods which contain and use a number of different error correcting agents. Such error correcting agents will have activity related to the correction of one or more of the following error types, deletions, insertion and substitution, also referred to as mismatches. Further, with respect to substitutions, activity will generally be directed to different types of substitutions.

A number of different polymerases and types of polymerases may be contained and used in compositions and methods set out herein. It is believed that the type of polymerase used in one or more steps of assembly PCR and amplification workflows affect the number of errors present in assembled nucleic acid molecules.

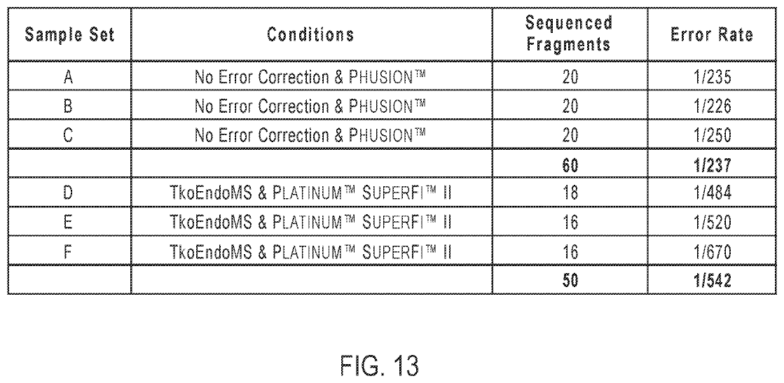

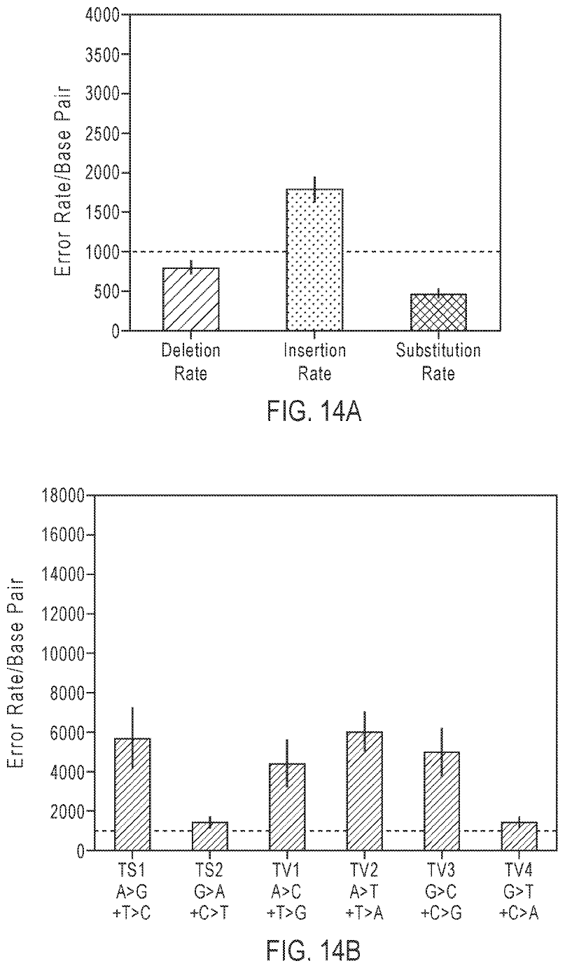

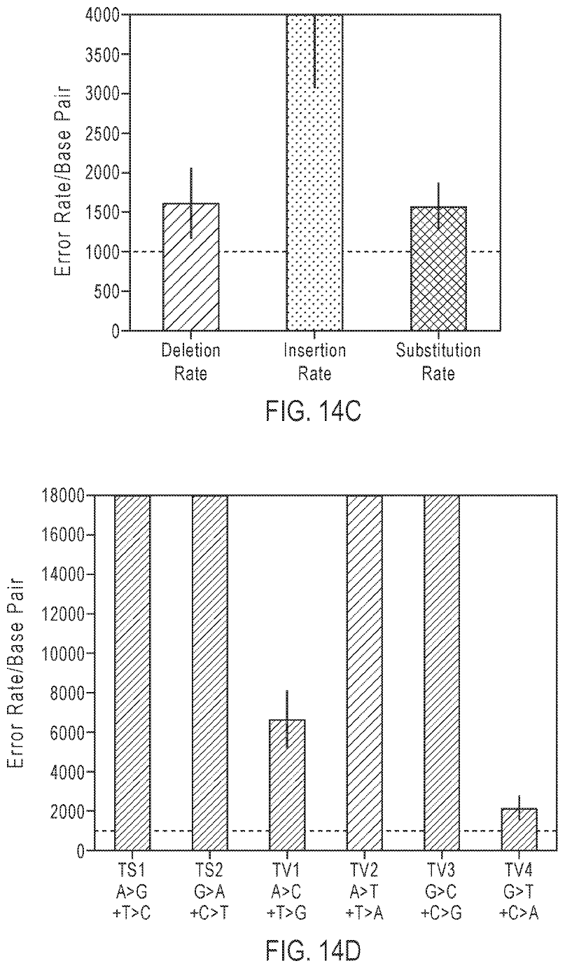

FIGS. 13 and 14A-14D show data generated using different type of polymerases. FIG. 13 shows data generated using no error correction in conjunction with PHUSION™ DNA polymerase and assembly PCR and amplification error correction was performed using TkoEndoMS in conjunction with PLATINUM™ SUPERFI™ II DNA polymerase reagent.

A representative workflow of methods provided herein is set out in FIG. 5A. In this workflow, three nucleic acid segments (referred to as “Subfragments”) are pooled and subjected to error correction using the enzyme T7 endonuclease I (“T7NI”) (FIG. 5A, Line 2). The three nucleic acid segments are then assembled by PCR (secondary assembly PCR) (FIG. 5A, Line 3) and then subjected to a second round of error correction (FIG. 5A, Line 4). After another round of PCR (tertiary assembly PCR) (Line 5), the resulting nucleic acid molecules are then screened for those that are full-length (FIG. 5A, Line 7). These nucleic acid molecules may then be screened for remaining errors by, for example, nucleotide sequencing.

Following synthesis oligonucleotides may be assembled (primary assembly PCR) into larger nucleic acid molecules in a stepwise manner and optionally, amplified. Methods used to assemble nucleic acid molecules may vary (see, e.g., FIGS. 1A and 1B). Further, error correction may be integrated into suitable assembly processes regardless of the method used. In many instances, error correction may be performed using mismatch recognition proteins (e.g., thermostable mismatch recognition proteins, such as mismatch binding proteins and mismatch endonucleases).

In some aspects, assembled nucleic acid molecule length may vary from about 20 base pairs to about 10,000 base pairs, from about 100 base pairs to about 5,000 base pairs, from about 150 base pairs to about 5,000 base pairs, from about 200 base pairs to about 5,000 base pairs, from about 250 base pairs to about 5,000 base pairs, from about 300 base pairs to about 5,000 base pairs, from about 350 base pairs to about 5,000 base pairs, from about 400 base pairs to about 5,000 base pairs, from about 500 base pairs to about 5,000 base pairs, from about 700 base pairs to about 5,000 base pairs, from about 800 base pairs to about 5,000 base pairs, from about 1,000 base pairs to about 5,000 base pairs, from about 100 base pairs to about 4,000 base pairs, from about 150 base pairs to about 4,000 base pairs, from about 200 base pairs to about 4,000 base pairs, from about 300 base pairs to about 4,000 base pairs, from about 500 base pairs to about 4,000 base pairs, from about 50 base pairs to about 3,000 base pairs, from about 100 base pairs to about 3,000 base pairs, from about 200 base pairs to about 3,000 base pairs, from about 250 base pairs to about 3,000 base pairs, from about 300 base pairs to about 3,000 base pairs, from about 400 base pairs to about 3,000 base pairs, from about 600 base pairs to about 3,000 base pairs, from about 800 base pairs to about 3,000 base pairs, from about 100 base pairs to about 2,000 base pairs, from about 200 base pairs to about 2,000 base pairs, from about 300 base pairs to about 1,500 base pairs, etc.

Any number of methods may be used for nucleic acid amplification and assembly. One exemplary method is described in Yang et al., Nucleic Acids Research 21:1889-1893 (1993) and U.S. Pat. No. 5,580,759. In the process described in Yang et al., a linear vector is mixed with double-stranded nucleic acid molecules which share sequence homology at the termini. An enzyme with exonuclease activity (i.e., T4 DNA polymerase, T5 exonuclease, T7 exonuclease, etc.) is added which generates single-stranded overhangs of all termini present in the mixture. The nucleic acid molecules having single stranded overhangs are then annealed and incubated with a DNA polymerase and deoxynucleotide triphosphates under condition which allow for the filling in of single-stranded gaps. Nicks in the resulting nucleic acid molecules may be repaired by introduction of the molecule into a cell or by the addition of ligase. Of course, depending on the application and workflow, the vector may be omitted. Further, the resulting nucleic acid molecules, or sub-portions thereof, may be amplified by polymerase chain reaction.

Other methods of nucleic acid assembly include those described in U.S. Patent Publication Nos. 2010/0062495 A1; 2007/0292954 A1; 2003/0152984 AA; and 2006/0115850 AA, in U.S. Pat. Nos. 6,083,726; 6,110,668; 5,624,827; 6,521,427; 5,869,644; and 6,495,318 and WO 2020/001783 A1.

A method for the isothermal assembly of nucleic acid molecules is set out in U.S. Patent Publication No. 2012/0053087. In one aspect of this method, nucleic acid molecules for assembly are contacted with a thermolabile protein with exonuclease activity (e.g., T5 polymerase) and optionally, a thermostable polymerase, and/or a thermostable ligase under conditions where the exonuclease activity decreases with time (e.g., 50° C.). The exonuclease “chews back” one strand of the nucleic acid molecules and, if there is sequence complementarity, nucleic acid molecules will anneal with each other. In one embodiment, a thermostable polymerase may be used to fill in gaps and a thermostable ligase may be provided to seal nicks. In another embodiment, the annealed nucleic acid product may be directly used to transform a host cell and gaps and nicks will be repaired “in vivo” by endogenous enzymatic activities of the transformed cell.

Single-stranded binding proteins, such as T4 gene 32 protein and RecA, as well as other nucleic acid binding or recombination proteins known in the art, may be included, for example, to facilitate the annealing of nucleic acid molecules.

In some instances, standard ligase-based joining of partially and fully assembled nucleic acid molecules may be employed. For example, assembled nucleic acid molecule may be generated with restriction enzyme sites near their termini. These nucleic acid molecules may then be treated with one of more suitably restrictions enzymes to generate, for example, either one or two “sticky ends”. These sticky end molecules may then be introduced into a vector by standard restriction enzyme-ligase methods. In instances where the inert nucleic acid molecules have only one sticky end, ligases may be used for blunt end ligation of the “non-sticky” terminus.

### Multiplex Assembly of Nucleic Acid Molecules

The complexity of a population of oligonucleotides is, in part, determined by the number of different oligonucleotides present. In some instances, the number of oligonucleotides present that are designed to have different nucleotide sequences, may be from about 2,000 to about 20,000 (e.g., from about 2,000 to about 20,000, from about 2,000 to about 20,000, from about 2,000 to about 20,000, from about 2,000 to about 20,000, from about 2,000 to about 20,000, from about 2,000 to about 20,000, from about 2,000 to about 20,000, from about 2,000 to about 20,000, from about 2,000 to about 20,000, from about 2,000 to about 20,000, from about 2,000 to about 20,000, from about 2,000 to about 20,000, from about 2,000 to about 20,000, from about 2,000 to about 20,000, from about 2,000 to about 20,000, from about 2,000 to about 20,000, etc.).

Further, oligonucleotides in a reaction mixture may represent subfragments of more than one larger nucleic acid molecule. By way of example, if it is desired to assemble three assembled nucleic acid molecules in one reaction mixture and ten oligonucleotides are required to assemble each of the assembled nucleic acid molecules, then the reaction mixture would initially contain at least thirty oligonucleotides.

Provided herein are compositions useful for and methods of assembling more than one assembled, error corrected nucleic acid. In some instances, the number assembled error corrected nucleic acid molecule generated by these methods will be from about two to about one hundred (e.g., from about two to about ninety, from about two to about eighty, from about two to about seventy, from about two to about fifty, from about five to about ninety, from about five to about sixty, from about eight to about ninety, from about eight to about fifty, from about eight to about thirty-five, from about ten to about ninety, from about two to about sixty, from about fifteen to about ninety, from about fifteen to about fifty-five, etc.).

### Polymerases and Polymerase Reagents

There are a number of different types of DNA polymerase. By way of example, many prokaryotic cells contain DNA polymerase Type I, II and III. DNA polymerases may or may not have proofreading activity. Proofreading DNA polymerases typically also have 3′ to 5′ exonuclease activity. Further DNA polymerases may be thermostable or non-thermostable.

While any type of DNA polymerase may be contained and used in compositions and methods set out herein, in many instances, proofreading polymerases will be employed herein. In some instances, DNA polymerases will be formulated for “hot start”, where the DNA polymerase is bound to antibodies that release the DNA polymerase upon heating.

DNA polymerases that may be contained and used in compositions and methods set out herein. Exemplary DNA polymerases and DNA polymerase reagents include Phi29 DNA polymerase or its derivatives, Bsm, Bst, T4, T7, DNA Pol I, or Klenow Fragment; or mutants, variants and derivatives thereof. Additional exemplary DNA polymerases and DNA polymerase reagents include Taq, Tbr, Tfl, Tth, Tli, Tfi, Tne, Tma, Pfu, Pwo, and Kod DNA polymerase, as well as VENT® DNA polymerase (New England Biolabs), DEEP VENT® DNA polymerase (New England Biolabs); PHUSION™ DNA polymerase; PHUSION™ U DNA polymerase; SUPERFI™ II DNA polymerase; SUPERFI™ U DNA Polymerase; or mutants, variants and derivatives thereof; and/or GoTaq G2 Hot Start Polymerase (Promega), ONETAQ® Hot Start DNA Polymerase (New England Biolabs), TAKARA TAQ™ DNA Polymerase Hot Start (Takara), KAPA2G Robust HotStart DNA Polymerase (KAPA), FASTSTART™ Taq DNA Polymerase (Sigma-Aldrich), HotStart Taq DNA Polymerase (New England Biolabs), Q5® DNA Polymerase (New England Biolabs), KAPA HiFi DNA Polymerase (Roche), PRIMESTAR@ Max DNA Polymerase (Takara), and PRIMESTAR® GXL DNA Polymerase (Takara).

In some instances, the DNA polymerase may comprise a chimeric DNA polymerase. Further, the chimeric DNA polymerase may comprise a sequence nonspecific double-stranded DNA (dsDNA) binding domain. In some instances, the dsDNA binding domain may comprise Sso7d from Sulfolobus solfataricus; Sac7d, Sac7a, Sac7b, and Sac7e from S. acidocaldarius; and Ssh7a and Ssh7b from Sulfolobus shibatae; Pae3192; Pae0384; Ape3192; HMf family archaeal histone domains; or an archaeal proliferating-cell nuclear antigen (PCNA) homolog. Additionally, DNA polymerases present in compositions and used in methods set out herein may also comprise exonuclease activity and/or an exonuclease domain.

Further, DNA polymerases that may be contained and used in compositions and methods set out herein include all or part of a DNA polymerase set out in Table 14, as well as modified forms of such polymerases (e.g., DNA polymerases that are at least 90%, at least 95%, or at least 97.5% identical to a DNA polymerase set out in Table 14).

PHUSION™ U DNA polymerase (Thermo Fisher Scientific, cat. no. F555S) is an engineered high fidelity enzyme developed using fusion technology. Due to a mutation in the dUTP binding pocket of PHUSION™ U, PHUSION™ U overcomes a limitation of proofreading enzymes in that it is able to incorporate dUTP and read through uracil present in DNA templates. In addition to this property, PHUSION™ U is capable of amplifying long amplicons up to 20 kb.

DNA polymerases that may be present in compositions and used in methods set out herein include those that have been modified to reduce the effect of inhibiting substances and/or are formulated with one or more compound that reduces the effect of inhibiting substances. As an example, PLATINUM™ II Taq Hot-Start DNA Polymerase (Thermo Fisher Scientific, cat. no. 14966001) is a “hot start” polymerase formulation where the DNA polymerase has been modified to reduce the effect of interfering compounds (e.g., humic acid, xylan, hemin, etc.). Further, this is formulated to allow for primer annealing at 60° C.

DNA polymerase reagents may also be formulated to lessen the effect of interfering compounds. One category of compounds that may be used in such formulations are “amines”. Amines have been found to improve (1) nucleic acid synthesis product yields and/or (2) tolerance to inhibitors of nucleic acid synthesis. Amine contain compounds that may be contained and used in compositions and methods set out herein including compounds comprising one or more amines of formula I:

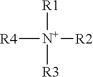

or salts thereof wherein R1 is H; R2 is chosen from alkyl, alkenyl, alkynyl, or (CH₂)n-R5, wherein n=1 to 3, and R5 is aryl, amino, thiol, mercaptan, phosphate, hydroxy, or alkoxy; and R3 and R4 may be the same or different and are independently chosen from H or alkyl, with the proviso that if R2 is (CH₂)n-R5, then at least one of R3 and/or R4 is alkyl.

Specific amine containing compounds that may be contained and used in compositions and methods set out herein include dimethylamine hydrochloride, diethylamine hydrochloride, diisopropylamine hydrochloride, ethyl(methyl)amine hydrochloride, and/or trimethylamine hydrochloride.

When one or more amine compounds are present in a formulation, the concentration of this or these compounds will generally be in the range of 5 mM to 500 mM (e.g., from about 5 mM to about 500 mM, from about 10 mM to about 500 mM, from about 20 mM to about 500 mM, from about 30 mM to about 500 mM, from about 40 mM to about 500 mM, from about 5 mM to about 300 mM, from about 5 mM to about 250 mM, from about 5 mM to about 200 mM, from about 5 mM to about 100 mM, from about 10 mM to about 250 mM, from about 20 mM to about 200 mM, from about 25 mM to about 180 mM, from about 50 mM to about 110 mM, etc.).

One specific example of a DNA polymerase reagent that may be used in methods set out herein is PLATINUM™ SUPERFI™ II DNA polymerase (Thermo Fisher Scientific, cat. no. 12361010).

### Vectors

Vectors that may be used in methods set out herein may be any vector suitable for cloning and transforming a host cell. In many instances, high-copy number vectors may be used to obtain high yields of the desired polynucleotide. Common high-copy number vectors include pUC (˜500-˜700 copies), PBLUESCRIPT@ or PGEM@ (˜300-˜500 copies, respectively) or derivatives thereof. In some instances, low-copy number vectors may be used, for example where high expression of a given insert may be toxic for the transformed cell. Such low-copy number vectors with copy numbers of between about 5 and about 30 include for example pBR322, various pET vectors, pGEX, pColE1, pR6K, pACYC or pSC101.

An exemplary list of vectors that can be used in any of the assembly or cloning methods disclosed herein, includes the following: BACULODIRECT™ Linear; DNA Cloning Fragment DNA; BACULODIRECT™ N-term Linear DNA; BACULODIRECT™ C-Term Baculovirus Linear DNA; BACULODIRECT™ N-Term Baculovirus Linear DNA; CHAMPION™ pET100/D-TOPO@; CHAMPION™ pET 101/D-TOPO®; CHAMPION™ pET104-DEST; CHAMPION™ pcDN3.1A/5-His-TOPO; pcDNA3.1(−); pcDNA3.1(+); pcDNA3.1(+)/myc-HisA; pcDNA3.1(+)/myc-His series; pcDNA3.1/His series; pcDNA3.1/Hygro(−); pcDNA3.1/Hygro(+); pcDNA3.1/NT-GFP-TOPO; pcDNA3.1/nV5-DEST; pcDNA3.1A/5-His series; pcDNA3.1/Zeo(+); pcDNA3.1/Zeo(+); pcDNA3.1DA/5-His-TOPO; pcDNA3.2/V5-DEST; pcDNA3.2-DEST; pcDNA4/His series; pcDNA4/HisMax-TOPO; pcDNA4/HisMax-TOPO; pcDNA4/myc-His series; pcDNA4/TO; pcDNA4/TO; pcDNA4/TO/myc-His series; pcDNA4/V5-His series; pcDNA5/FRT; pcDNA5/FRT/TO/CAT; pcDNA5/FRT/TO-TOPO; pcDNA-DEST47; pcDNA-DEST53; PDEST™10; PDEST™14; PDEST™15; pDEST™17; pDEST™20; pDEST™22; PDEST™24; pDEST™26; pDES™27; pDEST™32; pDEST™8; pDEST™38; pDEST™ 39; pDisplay; pDONR™ P2R P3; PDONR™ P2R-P3; pDONR™ P4-P1R; pDONR™ P4-P1R; pDONR™/Zeo; pDONR™201; pDONR™207; pDONR™221; pEF/myc/cyto; pEF/myc/mito; pEF/myc/nuc; pEFi/His series; pEF4/V5-His series; pEF5/FRT V5 D-TOPO; pEF5/FRT/V5-DEST™; pEF6/His series; pEF6/myc-His series; pEF6A/5-His-TOPO; pEF-DEST51; pENTR-TEV/D-TOPO; pENTR™/D-TOPO; pENTR™/D-TOPO; pHybLex/Zeo; pHyBLex/Zeo-MS2; pIB/His series; pIBA/5-His Topo; pYES2.1A/5-His-TOPO; pYES2/CT; pYES2/NT; pYES2/NT series; pYES3/CT; pYES6/CT; pYES-DEST™52; pYESTrp; pYESTrp2; pZeoSV2; pZeoSV2(+); pZErO-1; and pZErO-2.

In some aspects, the vector may have a limited size to allow for PCR-mediated elongation of the full-length fusion construct. Under certain conditions, full-length elongation and/or amplification of the fusion construct may not be required. In such circumstances, the size of the target vector may not be limiting. Thus, in some aspects the target vector may have a size of between about 0.5 and about 5 kb, or between about 1 kb and about 3 kb, whereas in other aspects the target vector may have a size of between about 2 kb and about 10 kb or between about 5 kb and about 20 kb.

Assembled nucleic acid molecules may also include functional elements which confer desirable properties. These elements may either be provided by the plurality of oligonucleotides or by the target vector. Examples of such elements include origins of replication, long terminal repeats, resistance markers (such as antibiotic resistance genes), selectable markers and antidote coding sequences (e.g., ccdA coding sequences for counter-acting toxic effects of ccdB), promoters, enhancers, polyadenylation signal coding sequences, 5′ and 3′ UTRs and other components suitable for the particular use(s) of the nucleic acid molecules (e.g., enhancing mRNA or protein production efficiency). In aspects where nucleic acid molecules are assembled to form an operon, the assembled nucleic acid products will often contain promoter and terminator sequences. Furthermore, assembled nucleic acid molecules may contain multiple cloning sites, such as, e.g., type II or type IIs cleavage sites and/or GATEWAY® recombination sites, as well as other sites for the connection of nucleic acid molecules to each other.

The vector may be linearized by any means including PCR amplification of a closed circular template vector molecule. Alternatively, the vector may be linearized by restriction enzyme cleavage with one or more enzymes producing either blunt or sticky ends. Such enzymes include restriction endonucleases of type II which cleave nucleic acid at fixed positions with respect to their recognition sequence. Restriction enzymes that can be selected to produce either “blunt” or “sticky” ends upon cleavage of a double-stranded nucleic acid are known to those skilled in the art and can be selected by the skilled person depending on the vector sequence and assembly requirements. In some instances, a vector may be linearized using a restriction endonuclease that generates blunt ends.

Following cleavage, the vector may either be used directly in, for example, an assembly PCR reaction (e.g., a sequence elongation and ligation reaction), or purified using gel extraction, or amplified in a PCR reaction prior to use in an assembly PCR reaction. Purification of a linearized vector generated by PCR amplification is often not required and the PCR product can be directly used in an assembly PCR reaction. Alternatively, a circular vector may be used comprising type IIS restriction enzyme cleavage sites and be subject to a one-step cleavage and ligation process to seamlessly clone one or more assembled nucleic acid molecules into the vector which is commonly known as Golden Gate cloning system as described below.

Following assembly PCR, the reaction mix comprising the assembled circularized construct or an aliquot thereof may be directly used to transform suitable competent host cells such as, e.g., a common E. coli strain according to standard protocols. The skilled person can select suitable host cells depending on construct size and nucleotide composition, plasmid copy number, selection criteria etc. Useful strains are available through the American Type Culture Collection and the E. coli Genetic Stock Center at Yale, as well as from commercial suppliers such as Agilent, Promega, Merck, Thermo Fisher Scientific, and New England Biolabs, respectively.

In many instances, nucleic acid molecules prepared by methods of provided herein will be replicable. Further, many of these replicable nucleic acid molecules will be circular (e.g., plasmids). Replicable nucleic acid molecules, regardless of whether they are circular, will generally be formed from the assembly of two or more (e.g., three, four, five, eight, ten, twelve, etc.) nucleic acid fragments. In some instances, methods provided herein employ selection based upon the reconstitution of one or more (e.g., two, three, four, etc.) selection marker or one or more (e.g., two, three, four, etc.) origin of replication resulting from the linking of different nucleic acid fragments. Further selection may result from the formation of a circular nucleic acid molecule, in instances where circularity is required for replication.

In an alternative embodiment, the single-stranded oligonucleotides used in a sequence elongation and ligation reaction (FIG. 1B) may be replaced by one or more double-stranded nucleic acid fragments with complementary ends to allow overlap extension PCR with a linearized target vector (and between fragments if two or more fragments are to be assembled into a target vector simultaneously). The complementary ends (i.e., the overlap) may have a size of between about 15 bp to about 50 bp, between about 20 bp to about 40 bp, such as, e.g., 40 bp. The size of the required overlap may depend on the size of the fragments to be fused and the melting temperatures thereof. The double-stranded fragment(s) is/are first assembled from single-stranded oligonucleotides and amplified in the presence of terminal primers as described above in steps (ii) and (iii), respectively, of a workflow such as that set out in FIG. 1A. The amplified fragments may then be subjected to one or more error correction and/or error removal rounds (e.g., by mismatch endonuclease treatment as described above) and subsequently used in a combined insertion, elongation reaction as described for the sequence elongation and ligation reaction above. In some aspects, overlaps of the interconnected adjacent fragments and/or overlaps of the terminal fragments to the linearized vector may be from about 15 to about 40 or from about 18 to about 30 nucleotides in length. In aspects where hybridization is required over a longer region to guarantee successful assembly, the overlaps may be from about 30 to about 60 nucleotides in length or even more than 60 nucleotides in length.

Assembled constructs obtained by an assembly workflow may be further combined with other assembly workflow products or nucleic acid molecules obtained from other sources to assemble larger nucleic acid molecules (e.g., genes). Constructs of larger sizes may be assembled by any means known to the person skilled in the art. For example, Type IIs restriction site mediated assembly methods may be used to assemble multiple fragments (e.g., two, three, five, eight, ten, etc.) when larger constructs are desired (e.g., 5 to 100 kilobases). One suitable cloning system is referred to as Golden Gate which is set out in various forms in U.S Patent Publication No. 2010/0291633 A1 and PCT Publication WO 2010/040531.

It may be desirable at a number of points during workflows of provided herein to separate nucleic acid molecules or assembly products from reaction mixture components (e.g., dNTPs, primers, truncated oligonucleotides, tRNA molecules, buffers, salts, proteins, etc.). This may be done in a number of ways, such as, e.g., by enzymatically removing undesired nucleic acid side-products with an exonuclease, restriction enzyme or UNG glycosylase as described above. In some instances, the nucleic acid molecules may be precipitated or bound to a solid support (e.g., magnetic beads). Once separated from reaction components for facilitating a process (e.g., pooling or multiplexing of selected oligonucleotides, nucleic acid synthesis, error correction, etc.), nucleic acid molecules may then be used in additional reactions (e.g., assembly PCR reactions, amplification, cloning etc.).

Larger nucleic acid molecules may also be assembled in vivo. In in vivo assembly methods, a mixture of all of the subfragments to be assembled is often used to transfect the host cell using standard transfection techniques. The ratio of the number of molecules of subfragments in the mixture to the number of cells in the culture to be transfected should be high enough to permit at least some of the cells to take up more molecules of subfragments than there are different subfragments in the mixture. Thus, in most instances, the higher the efficiency of transfection, the larger number of cells will be present which contain all of the nucleic acid subfragments required to form the final desired assembly product. Technical parameters along these lines are set out in U.S. Patent Publication No. 2009/0275086 A1.

Large nucleic acid molecules are relatively fragile and, thus, shear readily. One method for stabilizing such molecules is by maintaining them intracellularly. Thus, in some aspects, subject matter set out herein involves the assembly and/or maintenance of large nucleic acid molecules in host cells. Large nucleic acid molecules will typically be 20 kb or larger (e.g., larger than 25 kb, larger than 35 kb, larger than 50 kb, larger than 70 kb, larger than 85 kb, larger than 100 kb, larger than 200 kb, larger than 500 kb, larger than 700 kb, larger than 900 kb, etc.).

Methods for producing and even analyzing large nucleic acid molecules are known in the art. For example, Karas et al., “Assembly of eukaryotic algal chromosomes in yeast, Journal of Biological Engineering 7:30 (2013) shows the assembly of an algal chromosome in yeast and pulse-field gel analysis of such large nucleic acid molecules.

As suggested above, one group of organisms known to perform homologous recombination fairly efficient is yeasts. Thus, host cells used in the practice of methods set out herein may be yeast cells (e.g., Saccharomyces cerevisiae, Schizosaccharomyces pombe, Pichia, pastoris, etc.).

Yeast hosts are particularly suitable for manipulation of donor genomic material because of their unique set of genetic manipulation tools. The natural capacities of yeast cells, and decades of research have created a rich set of tools for manipulating DNA in yeast. These advantages are well known in the art. For example, yeast, with their rich genetic systems, can assemble and re-assemble nucleotide sequences by homologous recombination, a capability not shared by many readily available organisms. Yeast cells can be used to clone larger pieces of DNA, for example, entire cellular, organelle, and viral genomes that are not able to be cloned in other organisms. Thus, in some aspects, the enormous capacity of yeast genetics to generate large nucleic acid molecules (e.g., synthetic genomics) may be harnessed by using yeast as host cells for assembly and maintenance.

### EXAMPLES

### Example 1

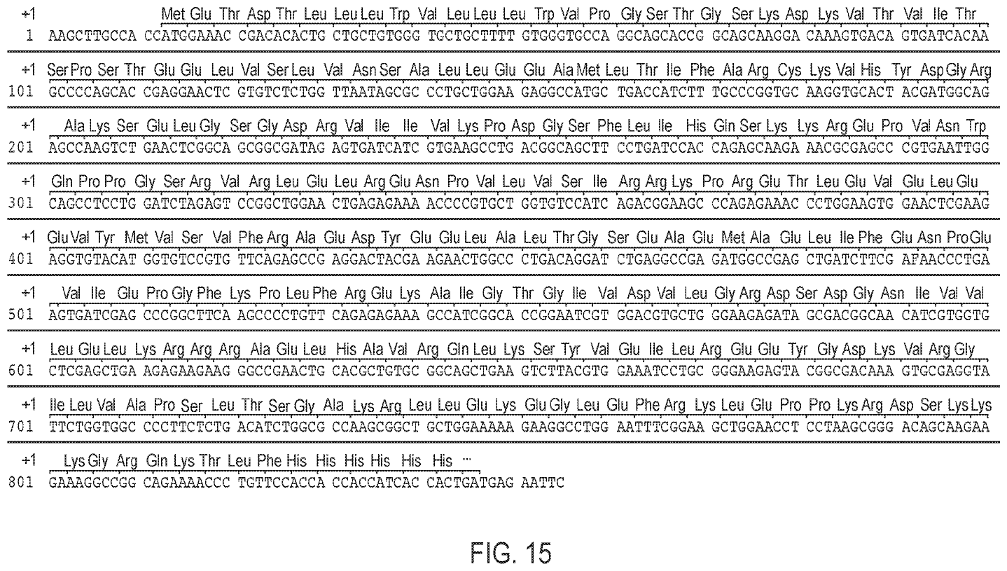

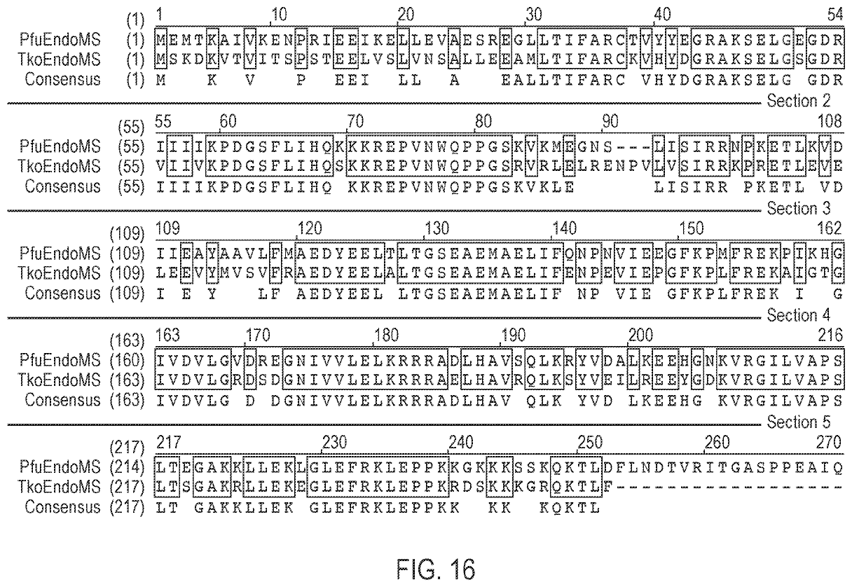

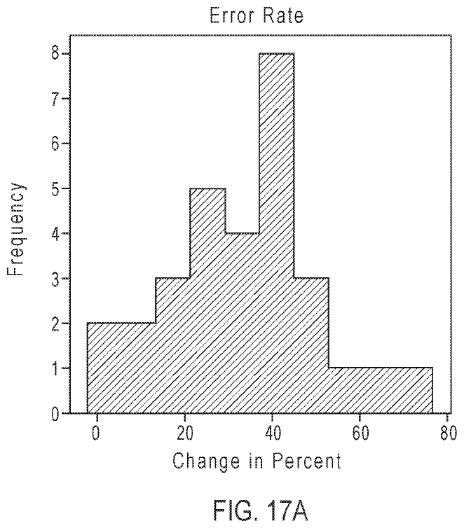

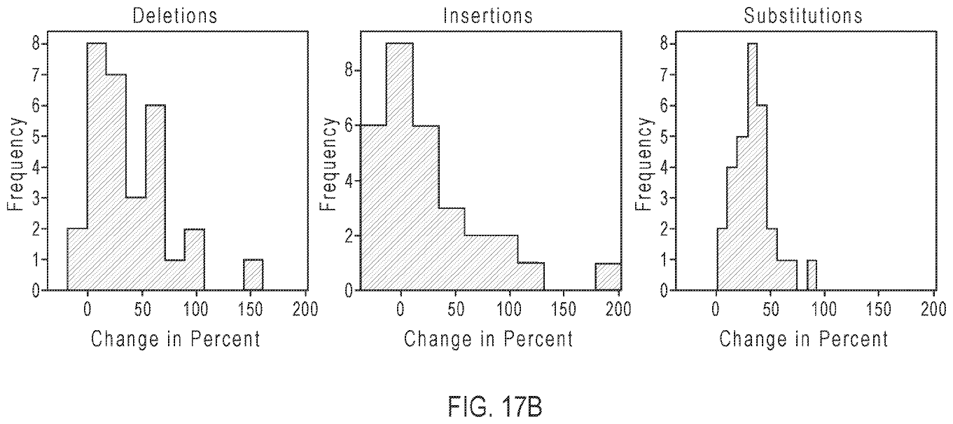

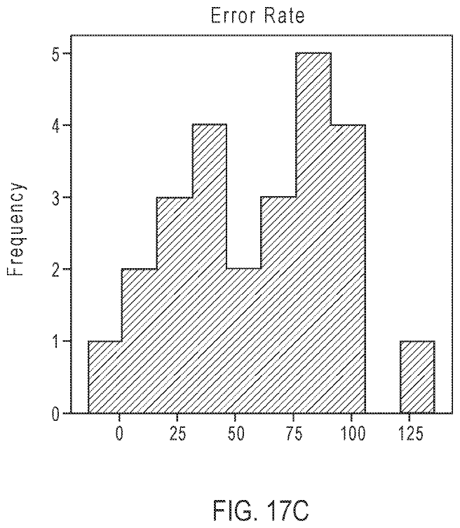

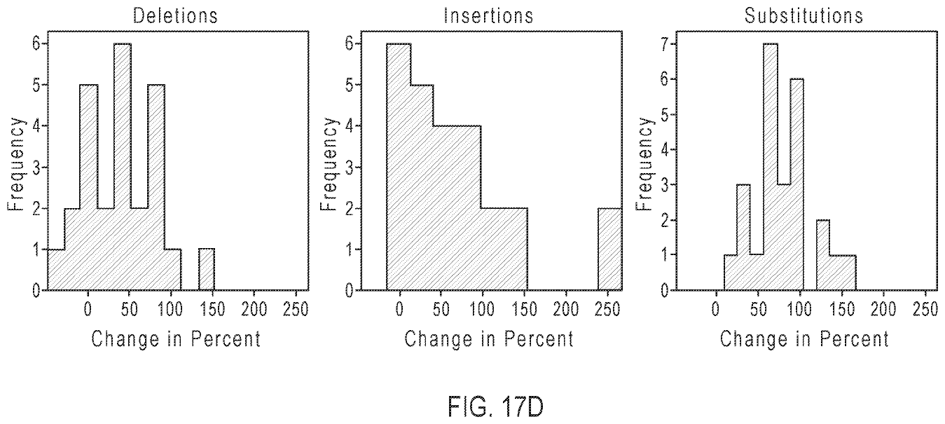

A codon optimized coding sequence for TkoEndoMS containing an amino terminal signal peptide (METDTLLLWV LLLWVPGSTG SKDKVTVIT (SEQ ID NO: 5)) and a carboxy terminal six histidine purification tag (FIG. 15) was designed using the follow parameters. The codon usage was adapted to the codon bias of Homo sapiens genes. In addition, regions of very high (>80%) or very low (<30%) GC content have been avoided where possible.

During the optimization process the following cis-acting sequence motifs were avoided where applicable: (1) internal TATA-boxes, chi-sites and ribosomal entry sites, (2) AT-rich or GC-rich sequence stretches, (3) RNA instability motifs, (4) repeat sequences and RNA secondary structures, and (5) (cryptic) splice donor and acceptor sites in higher eukaryotes. The result was the nucleotide sequence shown in FIG. 15, which encodes a protein having the amino acid sequence also shown in FIG. 15.

The nucleotide sequence set out in FIG. 15 was transfected into and expressed in EXPI™ 293 cells. EXPI™ 293 cells were cultured for six days after transfection, followed by harvesting of the expressed protein. Secreted TkoEndoMS protein was purified using the His tag by HisTrap column, using a linear gradient from 20-500 mM imidazole in Tris-HCl, 500 mM NaCl. Purified TkoEndoMS protein was dialyzed for 16 hours against 50 mM Tris-HCl pH 8.0, 0.5 mM DTT, 0.1 mM EDTA, 0.5 M NaCl. Purity was evaluated by Coomassie Blue staining and it was determined that the resulting TkoEndoMS was 95% pure. TkoEndoMS was stored at final concentration of 130 ng/4l in 50 mM Tris-HCl pH 8.0, 0.5 mM DTT, 0.1 mM EDTA, 0.5 M NaCl, 50% glycerol.

Benchmark Oligonucleotide Assembly Protocol

Assembly PCR

For 1 Reaction:

| Vol. | Reagent |
|---|---|
| 0.245 μl | 5x PHUSION ™ buffer HF, detergent-free |
| 0.025 μl | dNTPs (10 mM each) |
| 0.020 μl | PHUSION ™ DNA Polymerase |
| 0.440 μl | H₂O |
| 0.500 μl | 0.15 μM Oligonucleotide-Mix |
| 1.230 μl | Total Volume |

A master mix for all the reaction components was made except the mixture of oligonucleotides for assembly. 730 nl of the master mix was transferred to wells of a 384 well-plate using an ECHO® 555 Liquid Handler (Labcyte Inc.). 500 nl of the mixture of oligonucleotides was then added using an ECHO® 555 as well. Thermocycling was then performed using the cycler protocol set out below.

Cycler Protocol:

| | | | | | |
|---|---|---|---|---|---|
| 98° | C. | 4 | min | | |
| 98° | C. | 30 | sec | | |
| 54° | C.* | 30 | sec | {close oversize bracket} | 30 x |
| 65° | C. | 1 | min | | |
| 65° | C. | 4 | min | | |

| | | |
|---|---|---|
| 4° | C. | °° |

*Touch Down −0.8° C./Cycle

Amplification

For 1 Reaction:

| | |
|---|---|
| Vol. | Reagent |

| | | |
|---|---|---|
| 1.23 | μl | Assembly PCR Reaction Products |
| 1.753 | μl | 5x PHUSION ™ buffer HF, detergent-free |
| 0.175 | μl | dNTPs (10 mM each) |
| 0.140 | μl | PHUSION ™ DNA Polymerase |
| 0.088 | μl | 100 μM forward primer |
| 0.088 | μl | 100 μM reverse primer |
| 6.526 | μl | H₂O |
| 10.0 | μl | Total Volume |

A master mix of all the components except the assembly PCR products was prepared. 8.8 μl of the master mix was then transferred to wells of a 384 well-plate containing assembly PCR products with a multistep pipettor. Thermocycling was then performed using the cycler protocol set out below.

Cycler Protocol:

| | | | | | |
|---|---|---|---|---|---|
| 98° | C. | 4 | min | | |
| 98° | C. | 30 | sec | | |
| 58° | C. | 30 | sec | {close oversize bracket} | 30 x |
| 65° | C. | 1 | min | | |
| 65° | C. | 4 | min | | |

| | | |
|---|---|---|
| 4° | C. | °° |

EndoMS Oligonucleotide Assembly Protocol Using PHUSION™ DNA Polymerase

A. Assembly PCR

Identical to benchmark protocol, but reaction contains 0.020 μl TkoEndoMS (130 ng/μl). H₂O is 0.420 μl accordingly.

B. Amplification

Identical to benchmark protocol, but reaction contains 0.140 μl TkoEndoMS (130 ng/μl). H₂O is 6.386 μl accordingly.

Oligonucleotide Assembly Protocol Using SUPERFI™ II DNA Polymerase (EndoMS optional)

A. Assembly PCR

For 1 reaction without TkoEndoMS:

| Vol. | Reagent |
|---|---|
| 0.245 μl | 5x SUPERFI ™ II buffer |
| 0.025 μl | dNTPs (10 mM each) |
| 0.025 μl | SUPERFI ™ II DNA Polymerase |
| 0.435 μl | H₂O |
| 0.500 μl | 0.15 μM oligonucleotide-mix |
| 1.230 μl | Total Volume |

For 1 reaction with TkoEndoMS:

| Vol. | Reagent |
|---|---|
| 0.245 μl | 5x SUPERFI ™ II buffer |
| 0.025 μl | dNTPs (10 mM each) |
| 0.025 μl | SUPERFI ™ II DNA Polymerase |
| 0.020 μl | TkoEndoMS (130 ng/μl) |
| 0.415 μl | H₂O |
| 0.500 μl | 0.15 μM oligonucleotide-mix |
| 1.230 μl | Total Volume |

A master mix for all the reaction components was made except the mixture of oligonucleotides for assembly. 730 nl of the master mix was transferred to wells of a 384 well-plate using an ECHO® 555 Liquid Handler. 500 nl of the mixture of oligonucleotides was then added using an ECHO® 555 as well. Thermocycling was then performed using the cycler protocol set out below.

Cycler Protocol:

| | | | | | |
|---|---|---|---|---|---|
| 98° | C. | 30 | sec | | |
| 98° | C. | 5 | sec | | |
| 60° | C. | 15 | sec | {close oversize bracket} | 30 x |
| 68° | C. | 15 | sec | | |
| 68° | C. | 4 | min | | |

| | | |
|---|---|---|
| 4° | C. | °° |

B. Amplification

For 1 reaction without TkoEndoMS:

| | |
|---|---|
| Vol. | Reagent |

| | | |
|---|---|---|
| 1.23 | μl | Assembly PCR Reaction Products |
| 1.753 | μl | 5x SUPERFI ™ II buffer |
| 0.175 | μl | dNTPs (10 mM each) |
| 0.175 | μl | SUPERFI ™ II DNA Polymerase |
| 0.088 | μl | 100 μM forward primer |
| 0.088 | μl | 100 μM reverse primer |
| 6.491 | μl | H₂O |
| 10.0 | μl | Total Volume |

For 1 reaction with TkoEndoMS:

| | |
|---|---|
| Vol. | Reagent |

| | | |
|---|---|---|
| 1.23 | μl | Assembly PCR Reaction Products |
| 1.753 | μl | 5x SUPERFI ™ II buffer |
| 0.175 | μl | dNTPs (10 mM each) |
| 0.175 | μl | SUPERFI ™ II DNA Polymerase |
| 0.140 | μl | TkoEndoMS (130 ng/μl) |
| 0.088 | μl | 100 μM forward primer |
| 0.088 | μl | 100 μM reverse primer |
| 6.491 | μl | H₂O |
| 10.0 | μl | Total Volume |

A master mix of all the components except the assembly PCR products was prepared. 8.8 μl of the master mix was then transferred to wells of a 384 well-plate containing assembly PCR products with a multistep pipettor. Thermocycling was then performed using the cycler protocol set out below.

Cycler Protocol:

| | | | | | |
|---|---|---|---|---|---|
| 98° | C. | 30 | sec | | |
| 98° | C. | 5 | sec | | |
| 60° | C. | 15 | sec | {close oversize bracket} | 30 x |
| 68° | C. | 15 | sec | | |
| 68° | C. | 4 | min | | |

| | | |
|---|---|---|
| 4° | C. | °° |

Error Correction Protocol Using T7 Endonuclease I (T7NI)

A. Error Correction I (Denature and Re-Anneal)

For 1 reaction

| | |
|---|---|
| Amount | Reagent |

| | | |
|---|---|---|
| 200 | ng | Assembly PCR Products |
| 3.3 | μl | 10x AMPLIGASE ® buffer |
| 20 | μl | H₂O |

Cycler Protocol:

| | | |
|---|---|---|
| 98° | C. | 2 min |
| 4° | C. | 5 min |
| 37° | C. | 5 min |
| 4° | C. | °° |

Error Correction II (Mismatch cleavage)

For 1 reaction

| | |
|---|---|
| Amount | Reagent |

| | | |
|---|---|---|
| 6.0 | μl | Error Correction I Reaction Product |
| 1.0 | μl | T7 Endonuclease I |
| 1.0 | μl | Taq DNA Ligase |
| 0.25 | μl | 10x AMPLIGASE ® buffer |
| 1.75 | μl | H₂O |
| 10.0 | μl | Total Volume |

Cyler Protocol: 45° C., 20 min. in Cycler

B. Error Correction III (Amplification)

For 1 reaction

| | |
|---|---|
| Amount | Reagent |

| | | |
|---|---|---|
| 2.0 | μl | Error Correction II |
| 10.0 | μl | 5x PHUSION ™ buffer HF, detergent-free |
| 1.0 | μl | dNTPs (10 mM each) |
| 0.4 | μl | PHUSION ™ DNA Polymerase |
| 2.5 | μl | 10 μM fwd primer |
| 2.5 | μl | 10 μM rev primer |
| 31.6 | μl | H₂O |
| 50.0 | μl | Total Volume |

Cycler Protocol:

| | | | | | |
|---|---|---|---|---|---|
| 98° | C. | 4 | min | | |
| 98° | C. | 30 | sec | | |
| 58° | C. | 30 | sec | {close oversize bracket} | 30 x |
| 72° | C. | 1 | min | | |
| 72° | C. | 4 | min | | |

| | | |
|---|---|---|
| 4° | C. | °° |

### Example 2

Thermostable Mismatch Endonucleases (TsMMEs)

After having shown in Example 1 that the use of TkoEndoMS during assembly and/or amplification results in the generation of nucleic acid molecules with reduced error rates, conditions were tested for additional reduction of error rates. These conditions included the use of different thermostable mismatch endonucleases (abbreviated herein as “TsMMEs”), such as homologs of TkoEndoMS, different DNA polymerases, and different cycler protocols.

Materials and Methods:

“TsMMEs”, set out in Table 4, with the amino acid sequence of these enzymes shown in Table 15, and used in the experiments set out in this example were produced in Expi293 for thermostable error correction (abbreviated herein as “TsEC”). These enzymes produced by Thermo Fisher Scientific GeneArt GmbH (Regensburg, DE), were greater than 95% pure, and were each stored in the following buffer solution: 50 mM Tris-HCl pH 8.0, 0.5 mM DTT, 0.1 mM EDTA, 0.5 M NaCl, 50% glycerol.

No error correction using T7 endonuclease I was performed in experiments set out in this example.

TABLE 4
Thermostable Mismatch Endonucleases (“TsMMEs”)

| Enzyme | Protein Concentration | SEQ ID No |
|---|---|---|
| DmuEndoMS | 2.85 mg/ml | 6 |
| MjaEndoMS | 0.23 mg/ml | 7 |
| MkaNucS | 1.51 mg/ml | 8 |
| PabNucS | 0.44 mg/ml | 9 |
| PfuEndoMS | 7.61 mg/ml | 4 |
| PhoNucS | 0.19 mg/ml | 10 |
| PisEndoMS | 0.32 mg/ml | 11 |
| SacEndoMS | 0.36 mg/ml | 12 |
| TkoEndoMS | 0.13 mg/ml | 3 |

Benchmark Oligonucleotide Assembly Protocol

Benchmark data set out in this example was generated using PHUSION™ DNA polymerase and either no error correction or error correction mediated using specified thermostable enzymes. Unless stated otherwise, “Benchmark” data was generated using PHUSION™ DNA polymerase with no error correction. Benchmarking was done because oligonucleotides with different sequences contained different numbers of errors before error correction is performed. To correct for this variable, Benchmark data, unless stated herein otherwise, was generated using the same oligonucleotides used to generate comparative data.

Assembly PCR

For 1 Reaction:

| Vol. | Reagent |
|---|---|
| 0.245 μl | 5x PHUSION ™ buffer HF, detergent-free |
| 0.025 μl | dNTPs (10 mM each) |
| 0.020 μl | PHUSION ™ DNA Polymerase |
| 0.440 μl | H₂O |
| 0.500 μl | 0.15 μM Oligonucleotide-Mix |
| 1.230 μl | Total Volume |

A master mix was produced containing all the components except the Oligonucleotide-Mix. 730 nl of the master mix was transferred to individual wells of a 384 well-plate using a Labcyte ECHO® 555 Acoustic Liquid Handler. 500 nl of Oligonucleotide-Mix was then added to the same wells also using a Labcyte ECHO® 555 Acoustic Liquid Handler.

Cycler Protocol:

| | | | | | |
|---|---|---|---|---|---|
| 98° | C. | 4 | min | | |
| 98° | C. | 30 | sec | | |
| 54° | C.* | 30 | sec | {close oversize bracket} | 30 x |
| 65° | C. | 1 | min | | |
| 65° | C. | 4 | min | | |

| | | |
|---|---|---|
| 4° | C. | °° |

*Touch Down −0.8° C./Cycle

Amplification

For 1 Reaction:

| | |
|---|---|
| Vol. | Reagent |

| | | |
|---|---|---|
| 1.23 | ul | Assembly reaction |
| 1.753 | ul | 5x PHUSION ™ buffer HF, detergent-free |
| 0.175 | ul | dNTPs (10 mM each) |
| 0.140 | ul | PHUSION ™ DNA Polymerase |
| 0.088 | ul | 100 μM fwd primer |
| 0.088 | ul | 100 μM fwd primer |
| 6.526 | ul | H₂O |
| 10.0 | ul | Total Volume |

A master mix was prepared containing all the components except the assembly reaction product. 8.8 μl of this master mix was then transferred with a multistep pipettor to individual wells of a 384 well-plate containing the assembly reaction product.

Cycler Protocol:

| | | | | | |
|---|---|---|---|---|---|
| 98° | C. | 4 | min | | |
| 98° | C. | 30 | sec | | |
| 58° | C. | 30 | sec | {close oversize bracket} | 30 x |
| 65° | C. | 1 | min | | |
| 65° | C. | 4 | min | | |

| | | |
|---|---|---|
| 4° | C. | °° |

TsEC Oligonucleotide Assembly Protocol using PHUSION™ DNA Polymerase

Assembly

The methods used were identical to Benchmark Protocol set out earlier in this example but the reaction mixture contained 0.020 μl TkoEndoMS (130 ng/μl) and 0.420 μl of H₂O.

Amplification

The methods used were identical to the benchmark protocol set out above but the reaction mixture contained 0.140 μl TkoEndoMS (130 ng/μl) and 6.386 μl of H₂O.

Oligonucleotide Assembly Protocol using PLATINUM™ SUPERFI™ II DNA Polymerase (TsMME Optional)

For 1 Reaction without TsMME:

| Vol. | Reagent |
|---|---|
| 0.245 μl | 5x SUPERFI ™ II buffer |
| 0.025 μl | dNTPs (10 mM each) |
| 0.025 μl | SUPERFI ™ II DNA Polymerase |
| 0.435 μl | H₂O |
| 0.500 μl | 0.15 μM Oligonucleotide-Mix |
| 1.230 μl | Total Volume |

For 1 Reaction with TsMME:

| | |
|---|---|
| Vol. | Reagent |

| | | |
|---|---|---|
| 0.245 | μl | 5x SUPERFITM II buffer |
| 0.025 | μl | dNTPs (10 mM each) |
| 0.025 | μl | SUPER FITM II DNA Polymerase |
| x | μl | TsMME |
| 0.435-x | μl | H2O |
| 0.500 | μl | 0.15 uM Oligonucleotide-Mix |
| 1.230 | μl | Total Volume |

| | Amount per | Amount per |
|---|---|---|
| Enzyme | Assembly Reaction | Amplification Reaction |
| DmuEndoMS | 5.7 ng in x μl | 40 ng in y μl |
| MjaEndoMS | 4.6 ng in x μl | 32 ng in y μl |
| MkaNucS | 0.30 ng in x μl | 2.1 ng in y μl |
| PabNucS | 0.88 ng in x μl | 6.2 ng in y μl |
| PfuEndoMS | 0.61 ng in x μl | 4.3 ng in y μl |
| PhoNucS | 7.6 ng in x μl | 53 ng in y μl |
| PisEndoMS | 12.8 ng in x μl | 90 ng in y μl |
| SacEndoMS | 14.4 ng in x μl | 100 ng in y μl |
| TkoEndoMS | 0.52 ng in x μl | 3.6 ng in y μl |

A master mix was produced containing all the components except the Oligonucleotide-Mix. 730 nl of the master mix was transferred to individual wells of a 384 well-plate using a Labcyte ECHO® 555 Acoustic Liquid Handler. 500 nl of Oligonucleotide-Mix was then added to the same wells also using a Labcyte ECHO® 555 Acoustic Liquid Handler.

Cycler Protocol A

| | | | | | |
|---|---|---|---|---|---|
| 98° | C. | 30 | sec | | |
| 98° | C. | 5 | sec | | |
| 60° | C. | 15 | sec | {close oversize bracket} | 30 x |
| 68° | C. | 15 | sec | | |
| 68° | C. | 4 | min | | |

| | | |
|---|---|---|
| 4° | C. | °° |

Cycler Protocol B

| | | | | | |
|---|---|---|---|---|---|
| 98° | C. | 30 | sec | | |
| 98° | C. | 5 | sec | | |
| 60° | C. | 30 | sec | {close oversize bracket} | 25 x |
| 72° | C. | 30 | sec | | |
| 72° | C. | 2 | min | | |

| | | |
|---|---|---|
| 4° | C. | °° |

Cycler Protocol C

| | | | | | |
|---|---|---|---|---|---|
| 98° | C. | 30 | sec | | |
| 98° | C. | 5 | sec | | |
| | | | | {close oversize parenthesis} | 25x |
| 60° | C. | 48 | sec | | |
| 68° | C. | 2 | min | | |

| | | |
|---|---|---|
| 4° | C. | °° |

Cycler Protocol D

| | | | | | |
|---|---|---|---|---|---|
| 98° | C. | 30 | sec | | |
| 98° | C. | 5 | sec | | |
| 60° | C. | 30 | sec | {close oversize bracket} | 25 x |
| 80° | C. | 30 | sec | | |
| 72° | C. | 2 | min | | |

| | | |
|---|---|---|
| 4° | C. | °° |

Cycler Protocol E

| | | | | | |
|---|---|---|---|---|---|
| 98° | C. | 30 | sec | | |
| 98° | C. | 5 | sec | | |
| 60° | C. | 30 | sec | {close oversize bracket} | 25 x |
| 80° | C. | 30 | sec | | |
| 98° | C. | 5 | sec | | |
| 25° | C. | 1 | sec | {close oversize bracket} | 10 x |
| 80° | C. | 75 | sec | | |
| 72° | C. | 2 | min | | |

| | | |
|---|---|---|
| 4° | C. | °° |

Amplification

For 1 Reaction Without TsMME:

| | |
|---|---|
| Vol. | Reagent |

| | | |
|---|---|---|
| 1.23 | μl | Assembly Reaction Products |
| 1.753 | μl | 5x SUPERFI ™ II buffer |
| 0.175 | μl | dNTPs (10 mM each) |
| 0.175 | μl | SUPERFI ™ II DNA Polymerase |
| 0.088 | μl | 100 μM fwd primer |
| 0.088 | μl | 100 μM fwd primer |
| 6.491 | μl | H₂O |
| 10.0 | μl | Total Volume |

For 1 Reaction with TsMME:

| | |
|---|---|
| Vol. | Reagent |

| | | |
|---|---|---|
| 1.23 | μl | Assembly Reaction Products |
| 1.753 | μl | 5x SUPERFI ™ II buffer |
| 0.175 | μl | dNTPs (10 mM each) |
| 0.175 | μl | SUPERFI ™ II DNA Polymerase |
| y | μl | TsMME |
| 0.088 | μl | 100 μM fwd primer |
| 0.088 | μl | 100 μM fwd primer |
| 6.631- y | μl | H₂O |
| 10.0 | μl | Total Volume |

A master mix was prepared containing all the components except the assembly reaction product. 8.8 μl of this master mix was then transferred with a multistep pipettor to a well of a 384 well-plate containing the assembly reaction product.

Cycler Protocol:

| | | | | | |
|---|---|---|---|---|---|
| 98° | C. | 30 | sec | | |
| 98° | C. | 5 | sec | | |
| 60° | C. | 15 | sec | {close oversize bracket} | 30 x |
| 68° | C. | 15 | sec | | |
| 68° | C. | 4 | min | | |

| | | |
|---|---|---|
| 4° | C. | °° |

Results:

Assembly of 20 individual fragments using the “Benchmark Oligonucleotides Assembly Protocol” and PHUSION™ DNA polymerase (PHUSION™) was used to establish a “benchmark”/reference number of errors. The same 20 individual fragments were also assembled using the “Oligonucleotide Assembly Protocol” and PLATINUM™ SUPERFI™ II DNA Polymerase (“SUPERF^{I}™ II”) but with error correction using PhoNucS or SacEndoMS and Cycler Protocol C. The resulting data is shown below in Tables 5 and 6.

TABLE 5
Comparison of (1) Benchmark with (2) SUPERFI ™ II DNA
Polymerase with TsMME Error Correction. For (1), no error
correction was performed; for (2), TsMME was used in both
Assembly and Amplification.
The number of fragments for which an improvement in
the given range was achieved are shown

| PhoNucS | | | | | SacEndoMS | | | | |
|---|---|---|---|---|---|---|---|---|---|
| % Imp | ER | Del | Ins | Subs | % Imp | ER | Del | Ins | Subs |
| 0% | | 1 | | | 0% | | 5 | 4 | |
| 25% | | 1 | | | 25% | 1 | 7 | 9 | |
| 50% | | | | | 50% | 2 | 5 | 6 | |
| 75% | | 4 | | | 75% | 9 | 2 | 1 | |
| 100% | 2 | 3 | | 1 | 100% | 4 | 1 | | 4 |
| 125% | 2 | 2 | 2 | | 125% | 4 | | | 2 |
| 150% | 1 | 2 | 1 | 2 | 150% | | | | 3 |
| 175% | 3 | 2 | 1 | 1 | 175% | | | | 4 |
| 200% | 3 | | 5 | 1 | 200% | | | | 2 |
| 225% | 3 | 2 | | 4 | 225% | | | | 3 |
| 250% | 2 | 2 | 3 | 3 | 250% | | | | 1 |
| 275% | 3 | | 1 | 3 | 275% | | | | |
| 300% | | | 1 | 1 | 300% | | | | |
| 325% | 1 | | 1 | 1 | 325% | | | | |
| 350% | | | 1 | 2 | 350% | | | | |
| 375% | | | | | 375% | | | | |
| 400% | | | | | 400% | | | | |
| 425% | | 1 | | | 425% | | | | |
| 450% | | | 2 | | 450% | | | | |
| 475% | | | | | 475% | | | | 1 |
| 500% | | | 1 | | | | | | |
| 525% | | | | | | | | | |
| 550% | | | | | | | | | |
| 575% | | | | | | | | | |
| 600% | | | | | | | | | |
| 625% | | | | 1 | | | | | |
| 650% | | | | | | | | | |
| 675% | | | | | | | | | |
| 700% | | | 1 | | | | | | |
| Avg. for all | 217% | 155% | 273% | 272% | Avg. for all | 96% | 45% | 40% | 193% |
| fragments | | | | | fragments | | | | |

Key:
% Imp = % Improvement compared to benchmark,
ER = overall Error Rate,
Del = Deletion rate,
Ins = Insertion rate,
Subs = Substitution rate

The data set out in Table 5 indicates that processing with SUPERF^{I}™ 11 and PhoNucS results in a greater average improvement in overall error rate than processing with SUPERFI™ 11 and SacEndoMS. While SacEndoMS primarily corrects substitutions and has a smaller effect on deletions and insertions, PhoNucS was found to have significant error correction activity towards deletions and insertions, in addition to its higher activity towards substitutions. The data also indicates that sequence errors in some nucleic acid fragments are more readily correctable than in other fragments. For example, processing with SUPERFI™ II and PhoNucS results in 100% improvement in overall error rate for 2 fragments, and 275% improvement for 3 fragments, and processing with SUPERFI™ II and SacEndoMS results in 25% improvement in overall error rate for 1 fragment, and 100% improvement for 4 fragments. It is believed that this variability is partially due to sequence differences in the nucleic acid fragments. Nucleotide sequence difference can result in alteration in the prevalence of different error types in the nucleic acid fragments and, as discussed elsewhere herein, error correction enzymes differ in their ability to recognize and interact (e.g., bind to and/or cut) different error types.

TABLE 6
Comparison of (1) Benchmark with (2) SUPERFI ™ II DNA Polymerase
with TsMME Error Correction. For (1), no error correction was performed;
for (2), TsMME was used in both Assembly and Amplification.
The table shows the ratio of substitution types
present in the DNA fragments from Table 5

| | | | % of | % of |
|---|---|---|---|---|
| | | % of | SUPERFI ™ | SUPERFI ™ |
| Substitution | Mismatch | Bench- | II & | II & |
| Type | formed | mark | PhoNucS | SacEndoMS |
| TS A > G + T > C | A/C, G/T | 8.1% | 0.4% | 0.5% |
| TS G > A + C > T | A/C, G/T | 32.3% | 0.6% | 0.6% |
| TV A > C + T > G | A/G, C/T | 10.5% | 28.2% | 27.6% |
| TV A > T + T > A | A/A, T/T | 7.7% | 0.1% | 0.6% |
| TV G > C + C > G | C/C, G/G | 9.3% | 0.3% | 0.2% |
| TV G > T + C > A | A/G, C/T | 32.2% | 70.4% | 70.4% |

The “Benchmark” in this table was generated using the Benchmark Protocol with no error correction. “TS” refers to transitions, “TV” refers to transversions, “Mismatch formed” refers to the mismatch formed if a DNA strand containing the given substitution type anneals with a wild-type strand

Data set out in Table 6 shows that nucleic acid molecules assembled and amplified using PLATINUM™ SUPERFI™ II DNA Polymerase with error correction mediated by PhoNucS and SacEndoMS enzymes are almost completely devoid of 4 of the 6 substitution types, while the benchmark samples contain significant amounts of all 6 substitution types. Upon hybridization with a wild-type molecule, substitutions that are removed by the enzymes form mismatches for which their homolog TkoEndoMS has significant cleavage activity (Ishino et al., Nucl. Acids Res. 44:2977-2989 (2016)).

Data set out in Table 6 also suggest that the PhoNucS and the SacEndoMS enzymes do not exhibit high levels of cleavage activity for (1) A>C and T>G and (2) G>T and C>A transversions. Upon hybridization with a wild-type molecule, these transversions form mismatches for which their homolog TkoEndoMS has low cleavage activity (Ishino et al., Nucl. Acids Res. 44:2977-2989 (2016)).

TABLE 7
SUPERFI ™ II vs PHUSION ™ (Benchmark)
DNA Polymerases: Error Rate Comparison

| | | | | Improvement |
|---|---|---|---|---|
| Cycler | Fragments | | SUPERFI ™ II | vs. |
| Protocol | Assembled | Benchmark | (Error Rate Avg.) | Benchmark |
| A | 746 | 1 in 252 ± 31 | 1 in 311 ± 35 | 23% ± 9% |
| C | 90 | 1 in 249 ± 56 | 1 in 319 ± 106 | 28% ± 14% |

Note:
No error correction was performed

Table 7 shows a comparison of error rate data of nucleic acid fragments assembly and amplification by SUPERFI™ I vs PHUSION™ DNA polymerases. Two different thermocycler protocols were used (protocols A and C). As can be seen from the data, in the two runs set out in Table 7, nucleic acid fragments assembly and amplification by SUPERFI™ II was found to result in lower error rates when compared to PHUSION™ DNA polymerase. The data also shows that the error rate improvements seen in Table 5 are likely in small part due to the use of SuPERFI™ II. This suggests that the majority of the error rate improvements seen in Table 5 are due to the use of the TsMMEs.

TABLE 8
PHUSION ™ DNA Polymerase Error Rate Comparison with TsEC

| | | | | Improvement |
|---|---|---|---|---|
| Cycler | Fragments | | heSC | vs. |
| Protocol | Assembled | Benchmark | (error rate avg.) | Benchmark |
| Bench- | 640 | 1 in 294 ± 58 | 1 in 508 ± 127 | 73% ± 28% |
| mark | | | | |

Experimental Parameters: TsMME was TkoEndoMS

As seen in Table 8, use of the Benchmark Oligonucleotide Assembly Protoco with PHUSION™ DNA polymerase and TkoEndoMS for error correction resulted in a substantial decrease in the number of sequence errors ingenerated product nucleic acid molecules.

TABLE 9
Comparison of (1) Benchmark with (2) PHUSION ™
DNA Polymerase with TsMME Error Correction. For (1), no
error correction was performed; for (2), TsMME
(TkoEndoMS) was used in both Assembly and Amplification.
The table shows the ratio of substitution types present in 60
DNA fragments processed in one experiment. Experimental
parameters were as in Table 8, the results from this
experiment are included in the data shown in Table 8

| Substitution Type | Mismatch formed | % of Benchmark | % of heSC |
|---|---|---|---|
| TS A > G + T > C | A/C, G/T | 5.1% | 1.0% |
| TS G > A + C > T | A/C, G/T | 29.8% | 2.0% |
| TV A > C + T > G | A/G, C/T | 12.4% | 21.5% |
| TV A > T + T > A | A/A, T/T | 7.7% | 0.8% |
| TV G > C + C > G | C/C, G/G | 10.2% | 0.5% |
| TV G > T + C > A | A/G, C/T | 34.8% | 74.2% |

The “Benchmark” in this table was generated using the Benchmark Protocol with no error correction. “TS” refers to transitions, “TV” refers to transversions, “Mismatch formed” refers to the mismatch formed if a DNA strand containing the given substitution type anneals with a wild-type strand

Data set out in Table 9 shows that nucleic acid molecules assembled and amplified using PHUSION™ DNA Polymerase with error correction mediated by the TkoEndoMS enzyme have greatly reduced ratios for 4 of the 6 substitution types compared to the benchmark samples, which contain significant amounts of all 6 substitution types. Upon hybridization with a wild-type molecule, substitutions that are removed by TkoEndoMS form mismatches for which the enzyme has significant cleavage activity (Ishino et al., Nucl. Acids Res. 44:2977-2989 (2016)).

Data set out in Table 9 also suggest that the TkoEndoMS enzyme does not exhibit high levels of cleavage activity for (1) A>C and T>G and (2) G>T and C>A transversions. Upon hybridization with a wild-type molecule, these transversions form mismatches for which TkoEndoMS has low cleavage activity (Ishino et al., Nucl. Acids Res. 44:2977-2989 (2016)).

TABLE 10
SUPERFI ™ II Error Rate with Thermostable Error Correction (TsEC) compared
to PHUSION ™ DNA polymerase without TsEC (Benchmark)

| | Cycler | Fragments | Benchmark | TsEC | Improvement |
|---|---|---|---|---|---|
| TsMME | Protocol | Assembled | (error rate avg.) | (error rate avg.) | vs. Benchmark |
| PabNucS | A | 173 | 1 in 254 ± 33 | 1 in 490 ± 137 | 93% ± 47% |
| PabNucS | B | 113 | 1 in 294 ± 38 | 1 in 403 ± 17 | 37% ± 18% |
| PabNucS | C | 98 | 1 in 214 ± 1 | 1 in 351 ± 12 | 64% ± 6% |
| PabNucS | D | 30 | 1 in 331 ± 64 | 1 in 460 ± 248 | 39% ± 64% |
| PabNucS | E | 69 | 1 in 295 ± 53 | 1 in 497 ± 31 | 69% ± 20% |
| PhoNucS | A | 115 | 1 in 248 ± 22 | 1 in 629 ± 129 | 153% ± 59% |
| PhoNucS | B | 103 | 1 in 285 ± 40 | 1 in 599 ± 49 | 110% ± 24% |
| PhoNucS | C | 91 | 1 in 222 ± 7 | 1 in 571 ± 102 | 157% ± 38% |
| PhoNucS | D | 33 | 1 in 303 ± 45 | 1 in 697 ± 271 | 130% ± 84% |
| PhoNucS | E | 79 | 1 in 278 ± 54 | 1 in 687 ± 69 | 147% ± 23% |
| PisEndoMS | A | 200 | 1 in 244 ± 45 | 1 in 413 ± 63 | 69% ± 12% |
| PisEndoMS | B | 145 | 1 in 275 ± 22 | 1 in 464 ± 32 | 69% ± 9% |
| PisEndoMS | C | 52 | 1 in 196 ± 45 | 1 in 341 ± 127 | 74% ± 43% |
| PisEndoMS | D | 76 | 1 in 279 ± 17 | 1 in 495 ± 20 | 77% ± 4% |
| PisEndoMS | E | 87 | 1 in 271 ± 33 | 1 in 510 ± 54 | 88% ± 3% |
| SacEndoMS | A | 227 | 1 in 241 ± 35 | 1 in 442 ± 111 | 83% ± 21% |
| SacEndoMS | B | 155 | 1 in 281 ± 33 | 1 in 449 ± 45 | 60% ± 5% |
| SacEndoMS | C | 52 | 1 in 162 ± 68 | 1 in 263 ± 127 | 63% ± 52% |
| SacEndoMS | D | 78 | 1 in 278 ± 27 | 1 in 463 ± 33 | 67% ± 4% |
| SacEndoMS | E | 81 | 1 in 284 ± 49 | 1 in 485 ± 49 | 71% ± 13% |
| TkoEndoMS | A | 657 | 1 in 269 ± 60 | 1 in 479 ± 115 | 78% ± 26% |
| TkoEndoMS | B | 165 | 1 in 272 ± 23 | 1 in 408 ± 34 | 50% ± 13% |
| TkoEndoMS | C | 41 | 1 in 205 ± 71 | 1 in 327 ± 140 | 59% ± 43% |
| TkoEndoMS | D | 83 | 1 in 283 ± 22 | 1 in 418 ± 71 | 48% ± 14% |
| TkoEndoMS | E | 87 | 1 in 262 ± 25 | 1 in 423 ± 30 | 61% ± 4% |
| DmuEndoMS | A | 23 | 1 in 287 ± 59 | 1 in 368 ± 110 | 28% ± 24% |
| MjaEndoMS | A | 26 | 1 in 314 ± 48 | 1 in 387 ± 97 | 23% ± 23% |
| MkaNucS | A | 197 | 1 in 233 ± 41 | 1 in 327 ± 62 | 41% ± 6% |
| MkaNucS | C | 103 | 1 in 198 ± 1 | 1 in 249 ± 2 | 26% ± 2% |
| PfuEndoMS | A | 28 | 1 in 306 ± 60 | 1 in 509 ± 168 | 66% ± 57% |

Note:
Standard deviations were calculated across the experiments for a specific condition

A number of effects are seen in Table 10, one is that the use of different thermostable error correction enzymes results in different error rates in product nucleic acid molecules after assembly and amplification. Also, the number of errors present in nucleic acid molecules after assembly and amplification varies to some extent with the cycler protocol used. Thus, two factors that may be varied to yield assembled and amplified nucleic acid molecules with low error rates are (1) the error correction enzyme (or error correction enzymes) used and (2) the manner by which nucleic acid molecule sub-components are assembled and amplified (e.g., thermocycler protocol, buffer and buffer components used/present, etc.).

TABLE 11
SUPERFI ™ II Error Rate with Thermostable Error Correction (TsEC) compared
to PHUSION ™ DNA polymerase without TsEC (Benchmark)

| heSC | Cycler | Fragments | | heSC | Improvement |
|---|---|---|---|---|---|
| Enzyme | Protocol | Assembled | Benchmark | (error rate avg.) | vs. Benchmark |
| PhoNucS | A | 28 | 1 in 1092 ± 216 | 1 in 2089 ± 583 | 93% ± 44% |
| TkoEndoMS | A | 28 | 1 in 1092 ± 216 | 1 in 2340 ± 605 | 116% ± 50% |

Data in Table 11 also show that efficient reduction of error rates can be achieved independently of the initial error rate. For assembly and amplification using SUPERFI™ II polymerase and PhoNucS, 2.1 to 2.6-fold error reduction was achieved when the benchmark error rate was between 1 in 222 and 1 in 303 (Table 10), and 1.9-fold error reduction was achieved when the benchmark error rate was 1 in 1092. For assembly and amplification using SUPERFI™ II polymerase and TkoEndoMS, 1.5 to 1.8-fold error reduction was achieved when the benchmark error rate was between 1 in 205 and 1 in 283 (Table 10), and 2.1-fold error reduction was achieved when the benchmark error rate was 1 in 1092.

While specific aspects of subject matter set out herein have been shown and described herein, it will be obvious to those skilled in the art that such aspects are provided by way of example only. Numerous variations, changes, and substitutions will now occur to those skilled in the art without departing subject matter set out herein. It should be understood that various alternatives to the aspects of subject matter set out herein described herein may be employed in practicing subject matter set out herein. It is intended that the following claims define the scope of subject matter set out herein and that methods and structures within the scope of these claims and their equivalents be covered thereby.

### Nucleotide and Amino Acid Sequences

TABLE 12

| Thermostable Mismatch Repair Endonuclease Amino Acid Sequences | SEQ ID |
|---|---|
| Thermococcus kodakarensis (TkoEndoMS) | 13 |
| MSKDKVTVIT SPSTEELVSL VNSALLEEAM LTIFARCKVH YDGRAKSELG 50 | |
| SGDRVIIVKP DGSFLIHQSK KREPVNWQPP GSRVRLELRE NPVLVSIRRK 100 | |
| PRETLEVELE EVYMVSVFRA EDYEELALTG SEAEMAELIF ENPEVIEPGF 150 | |
| KPLFREKAIG TGIVDVLGRD SDGNIVVLEL KRRRAELHAV RQLKSYVEIL 200 | |
| REEYGDKVRG ILVAPSLTSG AKRLLEKEGL EFRKLEPPKR DSKKKGRQKT 250 | |
| LF 252 | |
| Amino Acid Variants: S47A, A47S, N76A, A76N | |
| Pyrococcus furiosus (PfuEndoMS) | 14 |
| MEMTKAIVKE NPRIEEIKEL LEVAESREGL LTIFARCTVY YEGRAKSELG 50 | |
| EGDRIIIIKP DGSFLIHQKK KREPVNFQPP GSKVKMEGNS LISIRRNPKE 100 | |
| TLKVDIIEAY AAVLFMAEDY EELTLIGSEA EMAELIFQNP NVIEEGFKPM 150 | |
| FREKPIKHGI VDVLGVDREG NIVVLELKRR RADLHAVSQL KRYVDALKEE 200 | |
| HGNKVRGILV APSLTEGAKK LLEKLGLEFR KLEPPKKGKK KSSKQKTLDF 250 | |
| LNDTVRITGA SPPEAIQ 267 | |
| Amino Acid Variants: F77Y | |
| Thermococcus kodakarensis (TkoEndoMS with N-Terminal Signal | 1 |
| Peptide and C-Terminal Histidine purification Tag) | |
| METDTLLLWV LLLWVPGSTG SKDKVTVITS PSTEELVSLV NSALLEEAML 50 | |
| TIFARCKVHY DGRAKSELGS GDRVIIVKPD GSFLIHQSKK REPVNWQPPG 100 | |
| SRVRLELREN PVLVSIRRKP RETLEVELEE VYMVSVFRAE DYEELALTGS 150 | |
| EAEMAELIFE NPEVIEPGFK PLFREKAIGT GIVDVLGRDS DGNIVVLELK 200 | |
| RRRAELHAVR QLKSYVEILR EEYGDKVRGI LVAPSLTSGA KRLLEKEGLE 250 | |
| FRKLEPPKRD SKKKGRQKTL FHHHHHH 276 | |

NOTE:
Amino acids shown in this table with bold, underlining may be substituted with
indicated other amino acids

TABLE 13

| Thermostable Mismatch Binding Proteins Amino Acid Sequences | SEQ ID |
|---|---|
| Thermus aquaticus | 15 |
| MEGMLKGEGP GPLPPLLQQY VELRDQYPDY LLLFQVGDFY ECFGEDAERL 50 | |
| ARALGLVLTH KTSKDFTTPM AGIPLRAFEA YAERLLKMGF RLAVADQVEP 100 | |
| AEEAEGLVRR EVTQLLTPGT LLQESLLPRE ANYLAAIATG DGWGLAFLDV 150 | |
| STGEFKGTVL KSKSALYDEL FRHRPAEVLL APELLENGAF LDEFRKRFPV 200 | |
| MLSEAPFEPE GEGPLALRRA RGALLAYAQR TQGGALSLQP FRFYDPGAFM 250 | |
| RLPEATLRAL EVFEPLRGQD TLFSVLDETR TAPGRRLLQS WLRHPLLDRG 300 | |
| PLEARLDRVE GFVREGALRE GVRRLLYRLA DLERLATRLE LGRASPKDLG 350 | |
| ALRRSLQILP ELRALLGEEV GLPDLSPLKE ELEAALVEDP PLKVSEGGLI 400 | |
| REGYDPDLDA LRAAHREGVA YFLELEERER ERTGIPTLKV GYNAVFGYYL 450 | |
| EVTRPYYERV PKEYRPVQTL KDRQRYTLPE MKEKEREVYR LEALIRRREE 500 | |
| EVFLEVRERA KRQAEALREA ARILAELDVY AALAEVAVRY GYVRPRFGDR 550 | |
| LQIRAGRHPV VERRTEFVPN DLEMAHELVL ITGPNMAGKS TFLRQTALIA 600 | |
| LLAQVGSFVP AEEAHLPLFD GIYTRIGASD DLAGGKSTFM VEMEEVALIL 650 | |
| KEATENSLVL LDEVGRGTSS LDGVAIATAV AEALHERRAY TLFATHYFEL 700 | |
| TALGLPRLKN LHVAAREEAG GLVFYHQVLP GPASKSYGVE VAAMAGLPKE 750 | |
| VVARARALLQ AMAARREGAL DAVLERLLAL DPDRLTPLEA LRLLQELKAL 800 | |
| ALGAPLDTMKG 810 | |
| Meiothermus taiwanensis WR-220 | 16 |
| MGSMTLKGQG PGPLPPLLEQ YVELRDAYPD YLLLFQVGDF YEAFGEDAER 50 | |
| LSRALNLTLT HKTAKDFTTP MAGIPVRSVD VHLEKLLKLG FRVAVADQVE 100 | |
| LAEEADKLVR REVTQLLTPG TILRENLLKP EANYLAAIST GDGYGLALLD 150 | |
| VSTGEFRGSV LYSKSALYDE LFRFRPAEVL LAPELYHNPT FLQEFQRRFP 200 | |
| VMLSEEGFQD GVGKAALHKQ FDPLPAGLEH PALQRSAGAV LAYALRVQEN 250 | |
| GLPQVRSFVR YDPGAFMQLS EATLRTLEIF EPSFVGDRSE ERTLLGVLGL 300 | |
| TRTAPGRRLL RAWLRHPLVE EAPLQARLDA VEALVKDGVL RAEVRKVLYR 350 | |
| MHDLERLAAR LLAGRASPRD LAALQRSLAL LPELAGLLAG VGPLLSVSER 400 | |
| LPDLSQVAEQ IAAALVEDPP LKITDGGLIR EGFDPELDEL RQRAEEGRAW 450 | |
| IARLESEARE KTGIPNLKVG YNAVFGYYLE VTRPHYALVP KDWRALQTLK 500 | |
| DRMRFSTPEL KEQERRILQA ETEAVKREYA VFLELRERVA QAADEVRQAA 550 | |
| QVLAELDVYA ALAEAAVEYG YSRPRFSRDG TLQIVAGRHP VVERNNPFIP 600 | |
| NDLTMSPAAR LLILTGPNMA GKSTYLRQTA LIALLAQVGS FVPAESATLP 650 | |
| LFDRIYTRIG ASDDIAGGRS TFMVEMDELA GILQGATPRS LVLLDEIGRG 700 | |
| TSTYDGLALA WAACEYLHDQ VRAYTLFATH YFELTALPLR MAAARNAHVA 750 | |
| AKEEAGGLVF YHQVLPGPAS QSYGLEVARL AGLPQAVLQR ARSVLDSLEA 800 | |
| SQKGLSKEIL EELLQLDLAR TSPLEALLFL RRLQDQLRGL APQEAEAERL 850 | |
| SQV 853 | |
| Meiothermus silvanus | 17 |
| MILKGQGSGP LPPLLQQYVE LRDAYPDYLL LFQVGDFYEA FGEDAERLSR 50 | |
| ALGITLTHKT SKDFTTPMAG IPIRSADSHL ERLLKMGFRV GLAEQTEPVE 100 | |
| AAEGLVRREV TQLLTPGTLT RENLLRPDAN YLAAIATGEG YGVVFLEVST 150 | |
| GEFRGVVLYS KSALYDELFR NRPAEVLLAP ELYANEAFRE EFQRRFPLMV 200 | |
| SSGSFDPQGA RSALVQQFGM LPSGLDHPAL ERAAGAVLAY ARTTQAGALP 250 | |
| QVRGFARYDP SAYMQLSETT LRTLEVYDPS PVGSYLPVGE ERTLMGVLGL 300 | |
| TRTAPGRRLL KAWLRQPLLD EGPIQARLDA VEALVRDSVL REAVRRLLYR 350 | |
| IHDLERLAAR LAAGRSNARD LAALARSLGL LPELQGQLLA CEPLRVLAER 400 | |
| LPLLAEVVER ISAALVEEPP LKITEGGLIK DGFDATLDAH RERAEAGRSW 450 | |
| IAGLEAAERS RTGIPSLKVG YNQVMGYYLE VTRPYYAQVP SDWRIVATLK 500 | |
| DRQRYTRPDL REKEREILLA EEAGRKREYE VFQELREELS GQAERVREAA 550 | |
| LVLAELDVYA TLAEVAARHG YTRPRFSPDR LFIRAGRHPV VERHLEGRFI 600 | |
| ANDLEMGPEA RLLILTGPNM SGKSTYLRQT ALIALLGQIG SFVPAEEAVL 650 | |
| PIFDRIYTRI GAADDIAGGR STFMVEMEEL AQILQGATAR SLVLLDEIGR 700 | |
| GTSTYDGLSL AWAASEYLHD RIKALTLFAT HYFELTALPE TLPAARNYHV 750 | |
| AAREEVGGLV FYHQVLPGPA SKSYGLEVAR LAGLPPEVLG RAGQLLAGLE 800 | |
| ARRDDWSQAL AEELLALDLT RLTPLEALLK LQQLRERLYP VMVSEAAD 848 | |
| Methanothrix thermoacetophila | 18 |
| MGPRLRERLL SYFESEEMAL RAIADEDMND LRAAIGERHA IATVRAARGL 50 | |
| RYGVSPESFL ATDEAERIYR TILNRIAEHA NTAYAIMRIS TLFPSGSPEL 100 | |
| IKEMRSVSLR AMDLRRRLGD VRDLLRRIKP LRRRGAQRIK GRAIAARSPE 150 | |
| ELAMARSMGF DRLLDIHLAE SPGELRDIAS GYDHVIVLSD PGVPLPGVEI 200 | |
| AEQLDIWYIA PEAVLSFYTE NRDALEAAME LAAVLEERGI EHFEDLSKLR 250 | |
| NALRRLFDDE DQSDISRIDD LLKRLPAAVD SALAQANTEL RKRIETSSVT 300 | |
| LGGPDLLRAL GRGDMIRDVF ETQMHGIFKS VISEARARVA ADLNLKGEAI 350 | |
| WLEEIIPEEI KYPLEINHRA LRQLEIELRR RREAEGLRRK REIAGSLANM 400 | |
| DGLTANLIKK LIQLDFLYAI GDFAISCGLT MPELIHDPGI GFRDGRHLFI 450 | |
| QNPEPVSYSL GACGIREYTE KAAILSGVNS GGKTSLLELM AQIAILAHMG 500 | |
| LPVPASECRI SIFDELYFFA KSSGTLSAGA FESTMRKLSA LATEKRKLVL 550 | |
| ADELEAITEP GASARIIASI LDMVQENGSV ALFVSHLADE IRRFSRTAVR 600 | |
| VDGIEAEGLD ERNNLILSRS PRYNHLARST PELILDRLVR TTEGKERTFY 650 | |
| EALLTRFRNT QSRSEKITGN CARLQIDS 678 | |

TABLE 14

| DNA Polymerase Amino Acid Sequences | SEQ ID |
|---|---|
| DNA Polymerase 1: Pyrococcus furiosus | 19 |
| MILDVDYITE EGKPVIRLFK KENGKFKIEH DRTFRPYIYA LLRDDSKIEE 50 | |
| VKKITGERHG KIVRIVDVEK VEKKELGKPI TVWKLYLEHP QDVPTIREKV 100 | |
| REHPAVVDIF EYDIPFAKRY LIDKGLIPME GEEELKILAF DIETLYHEGE 150 | |
| EFGKGPIIMI SYADENEAKV ITWKNIDLPY VEVVSSEREM IKRFLRIIRE 200 | |
| KDPDIIVTYN GDSFDFPYLA KRAEKLGIKL TIGRDGSEPK MQRIGDMTAV 250 | |
| EVKGRIHFDL YHVITRTINL PTYTLEAVYE AIFGKPKEKV YADEIAKAWE 300 | |
| SGENLERVAK YSMEDAKATY ELGKEFLPME IQLSRLVGQP LWDVSRSSTG 350 | |
| NLVEWFLLRK AYERNEVAPN KPSEEEYQRR LRESYTGGFV KEPEKGLWEN 400 | |
| IVYLDFRALY PSIIITHNVS PDTLNLEGCK NYDIAPQVGH KFCKDIPGFI 450 | |
| PSLLGHLLEE RQKIKTKMKE TQDPIEKILL DYRQKAIKLL ANSFYGYYGY 500 | |
| AKARWYCKEC AESVTAWGRK YIELVWKELE EKFGFKVLYI DTDGLYATIP 550 | |
| GGESEEIKKK ALEFVKYINS KLPGLLELEY EGFYKRGFFV TKKRYAVIDE 600 | |
| EGKVITRGLE IVRRDWSEIA KETQARVLET ILKHGDVEEA VRIVKEVIQK 650 | |
| LANYEIPPEK LAIYEQITRP LHEYKAIGPH VAVAKKLAAK GVKIKPGMVI 700 | |
| GYIVLRGDGP ISNRAILAEE YDPKKHKYDA EYYIENQVLP AVLRILEGFG 750 | |
| YRKEDLRYQK TRQVGLTSWL NIKKS 765 | |
| Amino Acid Variants: A408S and/or R762Q and/or R762X, where X is | |
| selected from Q, N, H, S, T, Y, C, M, W, A, I, L, F, V, P, and G | |
| and, more specifically, where X is selected from Q and N. | |
| DNA Polymerase 2: Pyrococcus furiosus | 20 |
| MILDADYITE EGKPVIRLFK KENGEFKIEH DRIFRPYIYA LLKDDSKIEE 50 | |
| VKKITAERHG KIVRIVDAEK VEKKFLGRPI TVWRLYFEHP QDVPTIREKI 100 | |
| REHSAVVDIF EYDIPFAKRY LIDKGLIPME GDEELKLLAF DIETLYHEGE 150 | |
| EFGKGPIIMI SYADEEEAKV ITWKKIDLPY VEVVSSEREM IKRFLKIIRE 200 | |
| KDPDIIITYN GDSFDLPYLA KRAEKLGIKL TIGRDGSEPK MQRIGDMTAV 250 | |
| EVKGRIHFDL YHVIRRTINL PTYTLEAVYE AIFGKPKEKV YADEIAKAWE 300 | |
| TGEGLERVAK YSMEDAKATY ELGKEFFPME AQLSRLVGQP LWDVSRSSTG 350 | |
| NLVEWFLLRK AYERNELAPN KPDEREYERR LRESYAGGFV KEPEKGLWEN 400 | |
| IVSLDFRSLY PSIIITHNVS PDTLNREGCR NYDVAPEVGH KFCKDFPGFI 450 | |
| PSLLKRLLDE RQKIKTKMKA SQDPIEKIML DYRQRAIKIL ANSYYGYYGY 500 | |
| AKARWYCKEC AESVTAWGRE YIEFVWKELE EKFGFKVLYI DIDGLYATIP 550 | |
| GGKSEEIKKK ALEFVDYINA KLPGLLELEY EGFYKRGFFV TKKKYALIDE 600 | |
| EGKIITRGLE IVRRDWSEIA KETQARVLEA ILKHGNVEEA VRIVKEVTQK 650 | |
| LSKYEIPPEK LAIYEQITRP LHEYKAIGPH VAVAKRLAAK GVKIKPGMVI 700 | |
| GYIVLRGDGP ISNRAILAEE YDPRKHKYDA EYYIENQVLP AVLRILEGFG 750 | |
| YRKEDLRWQK TQQTGL 766 | |
| Amino Acid Variants: A408S and/or R762Q and/or R762X, where X is | |
| selected from Q, N, H, S, T, Y, C, M, W, A, I, L, F, V, P, and | |
| G; in some aspects, X is selected from Q and N. | |
| DNA Polymerase 3: Pyrococcus DNA polymerase sequence with exonuclease | 21 |
| domain | |
| MILDADYITE EGKPVIRLFK KENGEFKIEH DRTFRPYIYA LLKDDSKIEE 50 | |
| VKKITAERHG KIVRIVDAEK VEKKFLGRPI TVWRLYFEHP QDVPTIREKI 100 | |
| REHSAVVDIF EYDIPFAKRY LIDKGLIPME GDEELKLLAF DIETLYHEGE 150 | |
| EFGKGPIIMI SYADEEEAKV ITWKKIDLPY VEVVSSEREM IKRFLKIIRE 200 | |
| KDPDIIITYN GDSFDLPYLA KRAEKLGIKL TIGRDGSEPK MQRIGDMTAV 250 | |
| EVKGRIHFDL YHVIRRTINL PTYTLEAVYE AIFGKPKEKV YADEIAKAWE 300 | |
| TGEGLERVAK YSMEDAKATY ELGKEFFPME AQLSRLVGQP LWDVSRSSTG 350 | |
| NLVEWFLLRK AYERNELAPN KPDEREYERR LRESYAGGFV KEPEKGLWEN 400 | |
| IVSLDFRSLY PSIIITHNVS PDTLNREGCR NYDVAPEVGH KFCKDFPGFI 450 | |
| PSLLKRLLDE RQKIKTKMKA SQDPIEKIML DYRQRAIKIL ANSYYGYYGY 500 | |
| AKARWYCKEC AESVTAWGRE YIEFVWKELE EKFGFKVLYI DTDGLYATIP 550 | |
| GGKSEEIKKK ALEFVDYINA KLPGLLELEY EGFYKRGFFV TKKKYALIDE 600 | |
| EGKIITRGLE IVRRDWSEIA KETQARVLEA ILKHGNVEEA VRIVKEVTQK 650 | |
| LSKYEIPPEK LAIYEQITRP LHEYKAIGPH VAVAKRLAAK GVKIKPGMVI 700 | |
| GYIVLRGDGP ISNRAILAEE YDPRKHKYDA EYYIENQVLP AVLRILEGFG 750 | |
| YRKEDLRWQK TXQTGLTSWL NIKKSGTGGG GATVKFKYKG EEKEVDISKI 800 | |
| KKVWRVGKMI SFTYDEGGGK TGRGAVSEKD APKELLQMLE KQKK 844 | |
| Amino Acid Variants: X is selected from Q, N, H, S, T, Y, C, M, | |
| W, A, I, L, F, V, P, and G; in some specific instances, X is | |
| selected from Q and N. | |
| DNA Polymerase 4: Pyrococcus DNA polymerase sequence with exonuclease | 22 |
| domain | |
| MILDADYITE EGKPVIRLFK KENGEFKIEH DRTFRPYIYA LLKDDSKIEE 50 | |
| VKKITAERHG KIVRIVDAEK VEKKFLGRPI TVWRLYFEHP QDVPTIREKI 100 | |
| REHSAVVDIF EYDIPFAKRY LIDKGLIPME GDEELKLLAF DIETLYHEGE 150 | |
| EFGKGPIIMI SYADEEEAKV ITWKKIDLPY VEVVSSEREM IKRFLKIIRE 200 | |
| KDPDIIITYN GDSFDLPYLA KRAEKLGIKL TIGRDGSEPK MQRIGDMTAV 250 | |
| EVKGRIHFDL YHVIRRTINL PTYTLEAVYE AIFGKPKEKV YADEIAKAWE 300 | |
| TGEGLERVAK YSMEDAKATY ELGKEFFPME AQLSRLVGQP LWDVSRSSTG 350 | |
| NLVEWFLLRK AYERNELAPN KPDEREYERR LRESYAGGFV KEPEKGLWEN 400 | |
| IVSLDFRSLY PSIIITHNVS PDTLNREGCR NYDVAPEVGH KFCKDFPGFI 450 | |
| PSLLKRLLDE RQKIKTKMKA SQDPIEKIML DYRQRAIKIL ANSYYGYYGY 500 | |
| AKARWYCKEC AESVTAWGRE YIEFVWKELE EKFGFKVLYI DTDGLYATIP 550 | |
| GGKSEEIKKK ALEFVDYINA KLPGLLELEY EGFYKRGFFV TKKKYALIDE 600 | |
| EGKIITRGLE IVRRDWSEIA KETQARVLEA ILKHGNVEEA VRIVKEVTQK 650 | |
| LSKYEIPPEK LAIYEQITRP LHEYKAIGPH VAVAKRLAAK GVKIKPGMVI 700 | |
| GYIVLRGDGP ISNRAILAEE YDPRKHKYDA EYYIENQVLP AVLRILEGFG 750 | |
| YRKEDLRWQK TQQTGLTSWL NIKKSGTGGG GATVKFKYKG EEKEVDISKI 800 | |
| KKVWRVGKMI SFTYDEGGGK TGRGAVSEKD APKELLQMLE KQKK 844 | |
| DNA Polymerase 5: Pyrococcus DNA polymerase sequence, K762X | 23 |
| MILDADYITE EGKPVIRLFK KENGEFKIEH DRTFRPYIYA LLKDDSKIEE 50 | |
| VKKITAERHG KIVRIVDAEK VEKKFLGRPI TVWRLYFEHP QDVPTIREKI 100 | |
| REHSAVVDIF EYDIPFAKRY LIDKGLIPME GDEELKLLAF DIETLYHEGE 150 | |
| EFGKGPIIMI SYADEEEAKV ITWKKIDLPY VEVVSSEREM IKRFLKIIRE 200 | |
| KDPDIIITYN GDSFDLPYLA KRAEKLGIKL TIGRDGSEPK MQRIGDMTAV 250 | |
| EVKGRIHFDL YHVIRRTINL PTYTLEAVYE AIFGKPKEKV YADEIAKAWE 300 | |
| TGEGLERVAK YSMEDAKATY ELGKEFFPME AQLSRLVGQP LWDVSRSSTG 350 | |
| NLVEWFLLRK AYERNELAPN KPDEREYERR LRESYAGGFV KEPEKGLWEN 400 | |
| IVSLDFRALY PSIIITHNVS PDTLNREGCR NYDVAPEVGH KFCKDFPGFI 450 | |
| PSLLKRLLDE RQKIKTKMKA SQDPIEKIML DYRQRAIKIL ANSYYGYYGY 500 | |
| AKARWYCKEC AESVTAWGRE YIEFVWKELE EKFGFKVLYI DIDGLYATIP 550 | |
| GGKSEEIKKK ALEFVDYINA KLPGLLELEY EGFYKRGFFV TKKKYALIDE 600 | |
| EGKIITRGLE IVRRDWSEIA KETQARVLEA ILKHGNVEEA VRIVKEVTQK 650 | |
| LSKYEIPPEK LAIYEQITRP LHEYKAIGPH VAVAKRLAAK GVKIKPGMVI 700 | |
| GYIVLRGDGP ISNRAILAEE YDPRKHKYDA EYYIENQVLP AVLRILEGFG 750 | |
| YRKEDLRWQK TXQTGL 766 | |
| Amino Acid Variants: X is selected from Q, N, H, S, T, Y, C, M, | |
| W, A, I, L, F, V, P, and G; in some instances, X is selected | |
| from Q and N. | |
| DNA Polymerase 6: Pyrococcus DNA polymerase sequence, A408S K762X | 24 |
| MILDADYITE EGKPVIRLFK KENGEFKIEH DRTFRPYIYA LLKDDSKIEE 50 | |
| VKKITAERHG KIVRIVDAEK VEKKFLGRPI TVWRLYFEHP QDVPTIREKI 100 | |
| REHSAVVDIF EYDIPFAKRY LIDKGLIPME GDEELKLLAF DIETLYHEGE 150 | |
| EFGKGPIIMI SYADEEEAKV ITWKKIDLPY VEVVSSEREM IKRFLKIIRE 200 | |
| KDPDIIITYN GDSFDLPYLA KRAEKLGIKL TIGRDGSEPK MQRIGDMTAV 250 | |
| EVKGRIHFDL YHVIRRTINL PTYTLEAVYE AIFGKPKEKV YADEIAKAWE 300 | |
| TGEGLERVAK YSMEDAKATY ELGKEFFPME AQLSRLVGQP LWDVSRSSTG 350 | |
| NLVEWFLLRK AYERNELAPN KPDEREYERR LRESYAGGFV KEPEKGLWEN 400 | |
| IVSLDFRSLY PSIIITHNVS PDTLNREGCR NYDVAPEVGH KFCKDFPGFI 450 | |
| PSLLKRLLDE RQKIKTKMKA SQDPIEKIML DYRQRAIKIL ANSYYGYYGY 500 | |
| AKARWYCKEC AESVTAWGRE YIEFVWKELE EKFGFKVLYI DTDGLYATIP 550 | |
| GGKSEEIKKK ALEFVDYINA KLPGLLELEY EGFYKRGFFV TKKKYALIDE 600 | |
| EGKIITRGLE IVRRDWSEIA KETQARVLEA ILKHGNVEEA VRIVKEVTQK 650 | |
| LSKYEIPPEK LAIYEQITRP LHEYKAIGPH VAVAKRLAAK GVKIKPGMVI 700 | |
| GYIVLRGDGP ISNRAILAEE YDPRKHKYDA EYYIENQVLP AVLRILEGFG 750 | |
| YRKEDLRWQK TXQTGL 766 | |
| Amino Acid Variants: X is selected from Q, N, H, S, T, Y, C, M, | |
| W, A, I, L, F, V, P, and G; in some instances, X is selected | |
| from Q and N. | |
| DNA Polymerase 7: Pyrococcus | |
| MILDADYITE EGKPVIRLFK KENGEFKIEH DRTFRPYIYA LLKDDSKIEE 50 | 25 |
| VKKITAERHG KIVRIVDAEK VEKKFLGRPI TVWRLYFEHP QDVPTIREKI 100 | |
| REHSAVVDIF EYDIPFAKRY LIDKGLIPME GDEELKLLAF DIETLYHEGE 150 | |
| EFGKGPIIMI SYADEEEAKV ITWKKIDLPY VEVVSSEREM IKRFLKIIRE 200 | |
| KDPDIIITYN GDSFDLPYLA KRAEKLGIKL TIGRDGSEPK MQRIGDMTAV 250 | |
| EVKGRIHFDL YHVIRRTINL PTYTLEAVYE AIFGKPKEKV YADEIAKAWE 300 | |
| TGEGLERVAK YSMEDAKATY ELGKEFFPME AQLSRLVGQP LWDVSRSSTG 350 | |
| NLVEWFLLRK AYERNELAPN KPDEREYERR LRESYAGGFV KEPEKGLWEN 400 | |
| IVSLDFRALY PSIIITHNVS PDTLNREGCR NYDVAPEVGH KFCKDFPGFI 450 | |
| PSLLKRLLDE RQKIKTKMKA SQDPIEKIML DYRQRAIKIL ANSYYGYYGY 500 | |
| AKARWYCKEC AESVTAWGRE YIEFVWKELE EKFGFKVLYI DTDGLYATIP 550 | |
| GGKSEEIKKK ALEFVDYINA KLPGLLELEY EGFYKRGFFV TKKKYALIDE 600 | |
| EGKIITRGLE IVRRDWSEIA KETQARVLEA ILKHGNVEEA VRIVKEVTQK 650 | |
| LSKYEIPPEK LAIYEQITRP LHEYKAIGPH VAVAKRLAAK GVKIKPGMVI 700 | |
| GYIVLRGDGP ISNRAILAEE YDPRKHKYDA EYYIENQVLP AVLRILEGFG 750 | |
| YRKEDLRWQK TQQTGL 766 | |

NOTE:
Amino acids shown in this table with bold, underlining may be substituted with indicated other
amino acids

TABLE 15
Exemplary Mismatch Recognition Proteins

| Gene/Protein Organism | Amino Acid Sequence | |
|---|---|---|
| Mismatch Activity* | | SEQ ID |
| TaqMutS | MGGMLKGEGPGPLPPLLQQYVELRDQYPDYLLLFQVGDFYECFGEDAER | 26 |
| Thermus aquaticus | LARALGLVLTHKTSKDFTTPMAGIPLRAFEAYAERLLKMGFRLAVADQVE | |
| Binding | PAEEAEGLVRREVTQLLTPGTLLQESLLPREANYLAAIATGDGWGLAFLDV | |
| | STGEFKGTVLKSKSALYDELFRHRPAEVLLAPELLENGAFLDEFRKRFPVML | |
| | SEAPFEPEGEGPLALRRARGALLAYAQRTQGGALSLQPFRFYDPGAFMRL | |
| | PEATLRALEVFEPLRGQDTLFSVLDETRTAPGRRLLQSWLRHPLLDRGPLE | |
| | ARLDRVEGFVREGALREGVRRLLYRLADLERLATRLELGRASPKDLGALRR | |
| | SLQILPELRALLGEEVGLPDLSPLKEELEAALVEDPPLKVSEGGLIREGYDPD | |
| | LDALRAAHREGVAYFLELEERERERTGIPTLKVGYNAVFGYYLEVTRPYYER | |
| | VPKEYRPVQTLKDRQRYTLPEMKEKEREVYRLEALIRRREEEVFLEVRERAK | |
| | RQAEALREAARILAELDVYAALAEVAVRYGYVRPRFGDRLQIRAGRHPVV | |
| | ERRTEFVPNDLEMAHELVLVTGPNMAGKSTFLRQTALIALLAQVGSFVPA | |
| | EEAHLPLFDGIYTRIGASDDLAGGKSTFMVEMEEVALILKEATENSLVLLDE | |
| | VGRGTSSLDGVAIATAVAEALHERRAYTLFATHYFELTALGLPRLKNLHVA | |
| | AREEAGGLVFYHQVLPGPASKSYGVEVAAMAGLPKEVVARARALLQAM | |
| | AARREGALDAVLERLLALDPDRLTPLEALRLLQELKALALGAPLDTMKG | |
| Tth MutS | MGGYGGVKMEGMLKGEGPGPLPPLLQQYVELRDRYPDYLLLFQVGDFY | 27 |
| Thermus thermophilus | ECFGEDAERLARALGLVLTHKTSKDFTTPMAGIPIRAFDAYAERLLKMGFR | |
| Binding | LAVADQVEPAEEAEGLVRREVTQLLTPGTLTQEALLPREANYLAAIATGD | |
| | GWGLAFLDVSTGEFKGTLLKSKSALYDELFRHRPAEVLLAPELRENEAFVA | |
| | EFRKRFPVMLSEAPFEPQGEGPLALRRAQGALLAYARATQGGALSVRPFR | |
| | LYDPGAFVRLPEASLKALEVFEPLRGQDTLFGVLDETRTAPGRRLLQAWLR | |
| | HPLLERGPLEARLDRVERFVREGALREGVRRLLFRLADLERLATRLELSRAS | |
| | PRDLAALRRSLEILPELKGLLGEEVGLPDLSGLLEELRAALVEDPPLKVSEGG | |
| | LIREGYDPDLDALRRAHAEGVAYFLDLEAREKERTGIPTLKVGYNAVFGYY | |
| | LEVTRPYYEKVPQEYRPVQTLKDRQRYTLPEMKERERELYRLEALIKRREEE | |
| | VFLALRERARKEAEALREAARILAELDVYAALAEVAVRHGYTRPRFGERLRI | |
| | RAGRHPVVERRTAFVPNDLEMAHELVLVTGPNMAGKSTFLRQTALIALL | |
| | AQIGSFVPAEEAELPLFDGIYTRIGASDDLAGGKSTFMVEMEEVALVLKEA | |
| | TERSLVLLDEVGRGTSSLDGVAIATALAEALHERRCYTLFATHYFELTALAL | |
| | PRLKNLHVAAKEEEGGLVFYHQVLPGPASKSYGVEVAEMAGLPKEVVER | |
| | ARALLSAMAARREGALEEVLERLLALDPDRLTPLEALRFLHELKALALGLPL | |
| | GSMKG | |
| AaeMutS1 | MEKSEKELTPMLSQYHYFKNQYPDCLLLFRLGDFYELFYEDAYIGSKELGLV | 28 |
| Aquifex aeolicus | LTSRPAGKGKERIPMCGVPYHSANSYIAKLVNKGYKVAICEQVEDPSKAK | |
| Binding | GIVKREVVRVITPGTFFERDTGGLASLYKKGNHYYVGYLNLAVGEFLGAKV | |
| | KIEELLDLLSKLNIKEILVKKGEKLPEELEKVLKVYVSELEEEFFEEGSEEILKDF | |
| | GVLSLQAFGFEEDTYSLPLGAVYKYAKTTQKGYTPLIPRPKPYRDEGFVRLD | |
| | IKAIKGLEILESLEGRKDISLFKVIDRTLTGMGRRRLKFRLLSPFRSREKIERIQ | |
| | EGVQELKENREALLKIRQILEGMADLERLVSKISSNMATPRELVYLKNSLKK | |
| | VEELRLLLLELKAPIFKEILQNFEDTKKIINDIEKTLVEDPPLHVKEGGLIREGV | |
| | NAYLDELRFIRDNAETYLREYEKKLRQETGIQSLKIGYNKVMGYYIEVTKPN | |
| | LKYVPSYFRRRQTLSNSERFTTEELQRLEEKILSAQTRINDLEYELYKELRERV | |
| | VKELDKVGNNASAVAEVDFIQSLAQIAYEKDWAKPQIHEGYELIIEEGRHP | |
| | VIEEFVENYVPNDTKLDRDSFIHVITGPNMAGKSSYIRQVGVLTLLSHIGSFI | |
| | PARRAKIPVVDALFTRIGSGDVLALGVSTFMNEMLEVSNILNNATEKSLVI | |
| | LDEVGRGTSTYDGIAISKAIVKYISEKLKAKTLLATHFLEITELEGKIEGVKNY | |
| | HMEVEKTPEGIRFLYILKEGKAEGSFGIEVAKLAGLPEEVVEEARKILRELEE | |
| | KENKKEDIVPLLEETFKKSEEAQRLEEYEEIIKKIEEIDIGNTTPLQALLILAELK | |
| | KKCSFSKKESGA | |
| AaeMutS2 | MREKDLIKLEFDKVKEVLASYAHSPATKEKIQNLKPYTNKEKVKEEIELSKAF | 29 |
| Aquifex aeolicus | FDIAENVRLFEFEDIRELLKKAKLQGAILGVEDILKILNVINLTKEIRRVLSSHV | |
| Binding | QRLEPLRKVYKKLYTFSPLENLIIGSIDPRGFVKDEASEELLRVRKSIRAVEEEI | |
| | KKRLDNLINRPDSAKFLSDRIVTIRNGRYVIPVKTSHVKKIFGIVHGTSSSGY | |
| | TTYVEPQFVIHLNNKLTELKQKEEEEVRKVLQRITEYIGDYAKELLESFEACV | |
| | EVDFQQCKYRFSKLVEGSFPDFGEWVELYEARHPVLVLVKEDVVPVGILLK | |
| | EKKGLILTGPNTGGKTVALKTLGLSVLMFQSAIPVPASPNSKLPLFEKVFTD | |
| | IGDEQSIEQNLSTFSAHVKNMAEFLPKSDENTLVLIDELGAGTDPIEGSAL | |
| | GIGILEYLKKKKAWVFVTTHHTPIKLYSTNSDYYTPASVLFDRETLKPLYKIA | |
| | YNTVGESMAFYIAQKYGIPSEVIEIAKRHVGEFGEQYIKAMEKLSDYVKKY | |
| | EEEFRKLEELRKELQKEKEEVEKLRKEYEEAKRKGWKEAYKEAREYLRKLVQ | |
| | ESEEIFKKAKEKKEIKEFVSKKREEIENLAPQKPQKLEVGDLVEFMGKKGKV | |
| | LEVKGNKALVLVDHLRMWLDTRELQKVGKAEPQKETKVTVQTPVMEKR | |
| | DTLNLIGKDVETAVRELEKFIEEAYSAGYKVVKVIHGIGSGKLKSAVREALSK | |
| | NEKVKFFRDAYPKEGGSGVTVVYLEYGEET | |
| AprMutS | MEELTPMMRQYYRIKERYKDALLFFRVGDFYELFDEDAKIASQELGIVLTS | 30 |
| Archaeoglobus profundus | RDKKHPMAGVPHHAVFPYIKRLIEKGYKVAICEQVEDPSKAKGLVRREVV | |
| Binding | RVITPGTLIEEELLTKENNYLMSIYKGRIYGIALIDVSTGEFLTTALESFDEVIA | |
| | EVLKFSPAECIVPEGFEELEELKKHVNVVHTLSQDEYSFKESLEILKECVQDF | |
| | ERLELEEECVRACGSALRYVKESLLIKTMKIRLQKYVSRDYMILDSTTLKNLE | |
| | VFRNLIDGSRRGTLIDVLDKTATAMGSRLLKRWLQRPLLNVDEIEKRLEAV | |
| | EELFEKSFLRQSLREVLREVYDLERIVSRIEYRKANARDLVALKNSLKAVEKI | |
| | KSFTFNSRRLKEIVEGLKALRDVVELIENAIVDNPPINIKDGGIIRDGYSREL | |
| | DELRRIKVDHENFIKNIEERERKATGIDKLKVGYNTVIGYYIEVPKSKLRFVP | |
| | KHYKRKQTLVNAERFTIPELEDIEEKVLACDEKIKALEYELFNEVREEVAKRV | |
| | DEIRECAFKIAELDVLSTFAEVAVLYNYTKPKVNDGYDIIIRDGRHPTVELTT | |
| | KFIPNDVNLTRDSRILIITGPNMAGKSTYLRMTALITIMAQIGCFVPASYAA | |
| | IGVVDRIFTRIGTVDDITRGYSSFMVEIDEVGKILKNATKRSLILLDEVGKST | |
| | GTKDGLSLAWAIIEYLHKIGAKTLFATHYHELSELESTLEGVKNYHFRIIEGE | |
| | TIEFDRKIKRGACTESYGIKIAEMVLPKEVIDRAYEIYRSLNIVNDDLMKEIA | |
| | KIDVNNLTPVQALVELDRIVRLCRSMKD | |
| CsaMutS1 | MQELTPMMQQYMEIKQRVKDCILFFRLGDFYEMFFDDAIIASKELEIALT | 31 |
| Caldicellulosiruptor | ARDCGNNEKAPMCGVPYHSAHSYIAKLIEKGYKVAICEQVEDPKLAKGVV | |
| saccharolyticus | KREITRIITPGTFIDENFSKANNFICCVARVESDFALTFVDISTGEMYACLIEN | |
| Binding | DIQKMINEISKYAPSEILISHLDNELYEVIRENYNSFVQRIEFIEIDRCYDLIDK | |
| | QMQITNINDKVALSVGNLLNYLVDTQKISFNYIKKFEFYRVQNYLQIDLSTK | |
| | RNLELTESIIARSKKNSLFGILDQAKTSMGSRLIKKWLERPLIDVVEINRRLD | |
| | AVEELYNNFPLLMQIEGLLEGIYDIERLSSKFAYKSINAKDLLSLKKSIEVLPR | |
| | LKELLGEFKSPLLKELYNELDTLEDVYSLIDSSINEDAPVGLKEGGIIKDGFND | |
| | HVDRLRNISKNSKELLIQYEEKERNLTGIKNLKIGYNKVFGYYIEVTKSNYSL | |
| | VPERYIRKQTLANAERYVTEELKKLEDEIINAEQKLVELEYELFCQIRDKIESQ | |
| | IERIQKTASCIAIIDALCSFAHIAIDNRYTKPIVYLGDRIYIKNGRHPVVEKMI | |
| | GYSNFVPNDTELDNDQNRVLIITGPNMAGKSTYMRQVALIVIMAQMGC | |
| | FVPAEEAQIGIVDKIFSRIGASDDISSGQSTFMVEMSEVANILKNATPKSLII | |
| | FDEVGRGTSTYDGLSIAWAVLEFVADKSKIGAKTLFATHYHELTELEEKISG | |
| | VKNYRVDVKEEGKNIIFLRKIVRGGCDSSYGIHVARLAGIPEEVLQRAEQIL | |
| | KKLEEADINRKEAKRLRKEIKREFTEQIEFFSYKKDEIIEKIENLDILNITPIQAL | |
| | NILSELKHEIIKAKERQLL | |
| CsaMutS2 | MNQKTLKALEYDKIVEILKNMAKSTPAKEYFENLIPSTNLADIENELNKVDE | 32 |
| Caldicellulosiruptor | GYRYVLKYGNPPTLEFENILPSLKKSKLGATLNPHEILQIGKVLKLSYEMRSY | |
| saccharolyticus | LSYTQDFSFLESMKKRLVNLKEVISRIDQTFLTADEILDTASPRLKEIRDRIRK | |
| Binding | LESRIRDELNSMIRDPKIQRFLQEPIITIRGEKLLLPVKAEFRNEVKGIVHDQ | |
| | SATGATLFVEPFVCVEISNQIRILKSQEKEEIERILQEISSLIASYCDEIETSFYA | |
| | LVELDIVFTKAIWAKEMNASKPVINTSGIINLKKARHPLIQKDKVVPIDIHL | |
| | GKDFDVLIITGPNTGGKTVTLKTVGLFCLLCQSGIFIPADEDSQLCIFQKIFA | |
| | DIGDDQSIVQSLSTFSAHMKNIIEITKNADDKTLVLLDEIGAGTDPEEGAAL | |
| | AKAILKYLSEKGSKVIATTHYGELKIFAQQEDRFENASCEFDVKTLKPTYRLL | |
| | IGIPGRSNALVISSNLGLDKGIVEMARGYLSQKTIDLDRIINEMEQKRKEAE | |
| | ENLELARKLKLEAQALKAAYEEEKKRFETERERIRKKAINEAKEIVERAQYEI | |
| | ENLFKDLRKLAENLKEKEVLKELEEKKREYERLIQSISQQEKQEAESKTKKTL | |
| | QNIRLGQKVYVRSFDAVGFVESLPDSKGNLTVQIGIMKLNVNISDIEEVEE | |
| | GEKKVYQTTSKNVKLREKSVDLSIDVRGKTSDDAILDVDKYLDDAYTSGLR | |
| | QVTIIHGKGTGVLRQAIRNFLKRHPLVKSFRDGTYGEGEQGVTIVELRD | |
| Dth MutS1 | MENMTPLYRQYKSIKDQFSDAILLFRLGDFYEAFEEDAKIISQELDIVLTSKE | 33 |
| Dictyoglomus | IGKGRRIPMAGVPYHALDSYLSKLVQKKYKVAICEQVEDPALAKGLVKREV | |
| thermophilum | VRVITPGTLVEDTLLEDKNNNFLSSIYALNKEYISLATIDVSTGEFFATEWRG | |
| Binding | KEAEEIIYSELVRLKPKEIILPFSLKDLFSELLTDLKREVDPKITLLDDNYFQFSD | |
| | YAIKYSDDKEKYPLAERSVNGALNYIKEVMFTIPTHIERVEIYQPQQYLILDS | |
| | TAIKHLELLETVREGQRRGSLIWVLDKTLTSMGARLLKKWILQPLLNVNAI | |
| | KKRQGAIKEFLEKEPWRREIEDILKEMPDLERINSRINYNTATPKELIYLRQA | |
| | LSFLPLLRKSLEKAESDRLKELKENLPDLEPLYEELDRALVESPPSHIKDGGYI | |
| | KDGYDPNLDELRRLLRESKDWLINLENRERERTGIKSLKIGYNQVFGYYIEV | |
| | TKANLNLVPPDYIRKQTLVNAERFITPELKEWENKILHAEDNIKKIEEELFQ | |
| | NLRKKVIEHSRDITTFAQIIGEIDVYISLAKAAREYNYVCPQVTNDYDVIIRE | |
| | GRHPVIERMLPPGTFVPNDAYLNREKFIDLITGPNMAGKSTYIRQIALIIILA | |
| | QMGSFIPAKEAKIGVVDRIFTRIGAWDDISSGESTFLVEMKEVGNILSHAT | |
| | ERSLIILDEVGRGTSTYDGISIAWAIVEYIHNKIKAKTLFATHYHELTELEKEL | |
| | RHLKNLSVAVQEKGKEIIFLHKIVDKPADKSYGIYVAQLADLPREVIERAEKI | |
| | LLELEKGREIKKKEVIQLPLFSEITDSKLEKLRNEILSLNTNELTPIQALLKIHE | |
| | WKELIK | |
| Dth MutS2 | MEKALKTLEWEKIIDEIEKKAETEGGKIRIRSLRPITDYQIIERWHLLNDEAF | 34 |
| Dictyoglomus | KTVSSFGYPSFSGIKNLEIYIDKAEKGGIVYPDEFEEIVRTIEIWSKLRDYQEK | |
| thermophilum | VRKIAPNLWRNTLHNLHDLYIQIRRCIQDGEVVDSASPELKQIRQKKERLN | |
| Binding | QKIKETLENIIQKEWRSYLQDQIITIRHGRYVVPIRQEFRGKIQGIVHDQSTS | |
| | GLTVYVEPQVIVELNNQIALLESEEKREIERILTRLTSILLSYKEEILENLRTSFE | |
| | LDFVYAKIKWAEKHKAITPILEKEKPLIILREARHPFLGEKAVPISLEVGRTEN | |
| | TLVITGPNTGGKTVTLKTIGLFVLLNQAGIPVPAKEGTVLGIFNQVFADIGD | |
| | EQSIEQNLSTFSSHMTNIISFIDYLERTGDKRVLILIDELGAGTDPQEGAALA | |
| | VALLEYFHEKGTINVIATHFPQLKVIASKYPGMENASMEFDEISLKPLYKVV | |
| | MGIPGKSNAILISKRLGLPRKILDRSLSLLSEDEIKLEEVIGELQRDRRRYEEEI | |
| | EKINKLKRDLQEEKRKIQEEKEMLEKEKAQLKAKYKEELFRDISKVEGKIREII | |
| | RKLQEESLTMKDAQSLQEELRNLRKELTIEEKREPENLTYIPHIGDRVLLRST | |
| | KKEGYVIDVDNEKKTALVQVGLLKINVPWAELAPSLKEEISVPSYVKVERV | |
| | NQEDVPREISIRMMTVDEGLEEVKKYLEKAFLAGLKRVRIVHGKGTGKLR | |
| | NAVHEYLSKVPYVKEYYLAPPNEGGEGATIVILDSPV | |
| MthMutS | MMNSRDSVGPILTEVGGIGERLARKIIDEFGGEDELLRAVKNLEIDRLVAIE | 35 |
| Methanothermobacter | GISQRRAIEIANKLLGNPLPSFLKTEKAFQIYRDILELMMSYAHTDYARNRI | |
| thermautotrophicus | MLLHPTTDTASMREHIRMVMEAKKMVEELPVEKLRGLLSNIDRPRDPPI | |
| Binding | NFNPSKAILVETREDYDLLIDMGLNRYHPVILNPDPGELDEYELIIYVYSEG | |
| | MLELEDTFSTIMVTADSDKHEIVPECVLNYFRKNLELFRNALEIKRILGMET | |
| | CLHEVVEIMDELQAAGGEEVDIDEVVNSVKAWADMKLRDLIKDIDLSGD | |
| | EVLTLLNQGMTAKLERIFDDVLSEARSMIKKRSGVEFDPFIKSYPLKIDQRE | |
| | VERVKRLEAASRNLREFEVKVDAATRLGELRGAVEGEIRDIMEFDYRLALG | |
| | LFAHEYELTEPEFGDEISLRGALHLNLVGSRKPQRVDYRIGDPDNVVLLTG | |
| | ANSGGKTTLLETLAQVTIMAQMGLPVCALEACVRVVDEVYFISKKRSLDA | |
| | GAFESFIRTFTPVTTSENSKLILLDELEAITELEAAVKIIATFIEFIGESSSLAVIV | |
| | THMAREIMKYVDVRVDGIEARGLDDDYNLIVDRTPRINYLARSTPELIIRM | |
| | IYEKSSDDERIVYGRILEKFQKTDKAEEQ | |
| PfuMutS | MKLRGDAREVYKRLLSRLESMIKLGEARTFLKKFEPTSDREEIIKRQNYLKE | 36 |
| Pyrococcus furiosus | GLKNVRDDLEEYLLSIRPIRFRREFFHDRILLVSDEEVEEAEKLDLCPVTSDP | |
| Binding | SEIEDYPLILSTIGYGIEVEVKPSHIAPELYIIPLWENRDVLEALSKVFPGGAA | |
| | DKILISLKEIEEIFKKMEILENLDEIIVEKEKELNRKIEEKLERFKLTLSGRDLVEF | |
| | MKALRAGNLEYLFHKFSALNDEIIEEINKAEKEISDVLGISVEIFPRDFPVEV | |
| | PPEQIEALKRELEREFKIEFYLKSRETVEKILPHLQKLKEEIQKAYELYFLLVVK | |
| | KFTRDFVFPEIVEEGIGFIEGRNLFIENPQPVSYFVGKSYGNFPGVEEANIVI | |
| | LTGANSGGKTSLLELISQIVILAHMGFPVPAKKAWFTVLDEIFFFKRKRSVY | |
| | GAGAFETSLKGLVRAIKGKGKKLILIDEFESITEPGAAAKILAELLKIAYEKGF | |
| | FVVIVSHLGEDLKREIPFARVDGIEAKGLDENLNLIVDRQPKFGVIGRSTPE | |
| | LIVERLARKGRGEEKMIMNRILKKFRK | |
| PyfMutS1 | MARVIVVDALAREHGKRMVTVDVIGVGPRAVAGLIEKLGVRASLYPLEAV | 37 |
| Pyrolobus fumarii | LEEPSILAEYDAMFVSAMSSDKPAAQRLAKLWKRFSNGPSVIGGPVSIEIG | |
| Binding | EALRLGYDYAVIGEAEVVLPNALKPMLKRDNDVLLHIKGVAFTRKGRIVFT | |
| | GKPPYASRETLNYIHYTDIRNYDAYWAARVYVEVVRGCSNWRRPRGRLP | |
| | DGRVCIHCEICTSAPLRARIKCPVSIQPGCGYCSVPVLYGPARSREVDTIVE | |
| | EVKRLIDLGVTRVVLSAPDFLDYKRDVLVEPEPLTDPCNPPANINAIRELLE | |
| | ALFDKIPEFAMREAYLMIENVKACLVDERVAKVLGEYLRGTPVHIGVETGS | |
| | DNHMWMLGRPITTTDAKRAVSLLRSAGLEPYVYFIYGLPWENSKVVHETI | |
| | MLMEELWKLGATKVTAYRFRPLPGTAFQDYEPPVPGSESYAILAKARELN | |
| | RAEKGRWLGRVVRGIVAGWHPAKKMLVVYPLPHGPVTLVRGPRGLIGW | |
| | LVKVEITGVISDRMLKGRIVSRIRRVARRSDGGLRVRSGASLSR | |
| PyfMutS2 | MVLFAAGNVGKLSDIPGVGPRIAEKLVEYFGDEEEALQAILSCRISLISEAIG | 38 |
| Pyrolobus fumarii | RAAATRLVHGLYRALFGAWPRDVAANDDAWKLFQAAKGVILKKVSSPA | |
| Binding | GRDVLACFLPMPSTGVKEAKRRLTLVKSLQEAIRELPRESIEALRSALSKLE | |
| | WPKPVKISRIKRTLIIIGDNVAYEKAKRTLSNLVDIVLVNDADEVDRVAAER | |
| | GEALVYDPDGVYAGPLPRVLELSVAEVVPEAIVEYFRVNWRVIEAVVDAL | |
| | EALGRWAPDILKIVGLDFDPTRVLNRLREALVFKGGEVAPDIDPEYQRLYT | |
| | ALTNLDRVVDEIELWVNNEVQTRLEKLEVRLSAAQFLRLFRVLREGGVEG | |
| | VDLPEEIYEVFEEVAEEAEKRVVEKLHLTPDEAEAMRGLVPRTPLFPIELNR | |
| | DKVEELRRVLRAKLSVKRFEVMQKLASKLVGVKAKLERLVDALALLDVLLA | |
| | QVELVGDGVAGVPEVSSEYLGVGFVDAIEASLVGKPNVQRISYVVGSTPY | |
| | KPDDTKGERVVLLTGANSGGKTTLLKTICETALLAQSGLVVLARQAWIGAF | |
| | DYVHFFSKPSGMVDAGALEATLRILAAIVAGGSGRRLVLVDELEAATEAG | |
| | AAARIMSVVVEKLCSRDNIVAVIVTHMAREILSNVKKVEGCIRVDGIEARG | |
| | LDENYNLIVDRNPRYYYLARSTPELVVRRLLYRAKGREEREFYETLLRVLSGA | |
| TbaMutS | MRLNSEARAIYKSIREEIKKRIQLKESLAYLDKFEPTNDKKEILRRQTYFKENF | 39 |
| Thermococcus barophilus | PRIVPELKGILAKIKPIRFKKDFLHDRLLVVDESELEKAQALGVCEVSTEPLE | |
| Binding | GYDLILSTVGIGIDVELSISEIAPELYIMPLWENRETLKALAQIANLLGEKSVA | |
| | ESILKKLGELEEIMKRRELIENLDELIAEEERRLNKKIAEKLERFSLTLTGKELL | |
| | EFLKELREGDYGAIFKHFSEIESEILEEINEAEKRLSERLNFTVELFSRENLYPI | |
| | EVLPESIEALHQELERELKIELYLKSREILDEIKHLIPKLKEEINKAYELEFLRAV | |
| | KEFTEGFIFPELIEGGITFINGRHLFIKNPQPVSYVVGKAPIEFNGVNGERVV | |
| | ILTGANSGGKTSLLELTSQIVILTHMGFPVNAEKAWVEVLDELFFFKRKRSI | |
| | YGAGAFETALKSFVRALTSDGRKLILIDEFEAITEPGAAVKIIAELLKIAYEKG | |
| | FYVIIVSHLGEDLKKELPFARVDGIEAQGLDENLNLIVDRQPKFGVLGKSTP | |
| | ELIVEKLYRTKRGKEKKIFERVWRSFL | |
| TmaMutS1 | MKVTPLMEQYLRIKEQYKDSILLFRLGDFYEAFFEDAKIVSKVLNIVLTRRQ | 40 |
| Thermotoga maritima | DAPMAGIPYHALNTYLKKLVEAGYKVAICDQMEEPSKSKKLIRREVTRVVT | |
| Binding | PGSIVEDEFLSETNNYMAVVSEEKGRYCTVFCDVSTGEVLVHESSDEQETL | |
| | DLLKNYSISQIICPEHLKSSLKERFPGVYTETISEWYFSDLEEVEKAYNLKDIH | |
| | HFELSPLALKALAALIKYVKYTMIAEDLNLKPPLLISQRDYMILDSATVENLS | |
| | LIPGDRGKNLFDVLNNTETPMGARLLKKWILHPLVDRKQIEERLKAVERLV | |
| | NDRVSLEEMRNLLSNVRDVERIVSRVEYNRSVPRDLVALRETLEIIPKLNEV | |
| | LSTFGVFKKLAFPEGLVDLLRKAIEDDPVGSPGEGKVIKRGFSSELDEYRDL | |
| | LEHAEERLKEFEEKERERTGIQKLRVGYNQVFGYYIEVTKANLDKIPDDYER | |
| | KQTLVNSERFITPELKEFETKIMAAKERIEELEKELFKSVCEEVKKHKEVLLEI | |
| | SEDLAKIDALSTLAYDAIMYNYTKPVFSEDRLEIKGGRHPVVERFTQNFVE | |
| | NDIYMDNEKRFVVITGPNMSGKSTFIRQVGLISLMAQIGSFVPAQKAILPV | |
| | FDRIFTRMGARDDLAGGRSTFLVEMNEMALILLKSTNKSLVLLDEVGRGT | |
| | STQDGVSIAWAISEELIKRGCKVLFATHFTELTELEKHFPQVQNKTILVKEE | |
| | GKNVIFTHKVVDGVADRSYGIEVAKIAGIPDRVINRAYEILERNFKNNTKK | |
| | NGKSNRFSQQIPLFPV | |
| TmaMutS2 | MDYLESLDFPKVVEIVKKYALSDLGRKHLDTLKPTVNPWDELELVEELLNY | 41 |
| Thermotoga maritima | FNRWGEPPIKGLNDISQEVEKVKSGSPLEPWELLRVSVFLEGCDILKKEFEK | |
| Binding | REYSRLKETFSRLSSFREFVEEVNRCIEQDGEISDRASPRLREIRTEKKRLSSE | |
| | IKRKADDFVRTHSQILQEQMYVYRDGRYLFPVKASMKNAVRGIVHHLSSS | |
| | GATVFLEPDEFVELNNRVRLLEEEERLEISRILRQLTNILLSRLNDLERNVELI | |
| | ARFDSLYARVKFAREFNGTVVKPSSRIRLVNARHPLIPKERVVPINLELPPN | |
| | KRGFIITGPNMGGKTVTVKTVGLFTALMMSGFPLPCDEGTELKVFPKIMA | |
| | DIGEEQSIEQSLSTFSSHMKKIVEIVKNADSDSLVILDELGSGTDPVEGAAL | |
| | AIAIIEDLLEKGATIFVTTHLTPVKVFAMNHPLLLNASMEFDPETLSPTYRVL | |
| | VGVPGGSHAFQIAEKLGLDKRIIENARSRLSREEMELEGLIRSLHEKISLLEE | |
| | EKRKLQKEREEYMKLREKYEEDYKKLRRMKIEEFDKELRELNDYIRKVKKEL | |
| | DQAIHVAKTGSVDEMREAVKTIEKEKKDLEQKRIEEATEEEIKPGDHVKM | |
| | EGGTSVGKVVEVKSGTALVDFGFLRLKVPVSKLRKTKKEEKKETSTFSYKPS | |
| | SFRTEIDIRGMTVEEAEPVVKKFIDDLMMNGISKGYIIHGKGTGKLASGV | |
| | WEILRKDKRVVSFRFGTPSEGGTGVTVVEVKV | |
| Tth MutS2 | MRDVLEVLEFPRVRALLAERAKTPLGRELALALAPLPREEAEKRHELTGEA | 42 |
| Thermus thermophilus | LSYPYALPEAGTLREAYGRALAGARLSGPELLKAAKALEEAMALKEELLPLK | |
| Binding | NALSQVAEGIGDHTPFLERVRKALDEEGAVKDEASPRLAQIRRELRPLRQ | |
| | QILDRLYALMDRHREAFQDRFVTLRRERYCVPVRAGMAQKVPGILLDESE | |
| | SGATLFIEPFSVVKLNNRLQALRLKEEEEVNRILRDLSERLAKDEGVPKTLEA | |
| | LGLLDLVQAQAALARDLGLSRPAFGERYELYRAFHPLIPDAVRNSFALDEK | |
| | NRILLISGPNMGGKTALLKTLGLAVLMAQSGLFVAAEKALLAWPDRVYAD | |
| | IGDEQSLQENLSTFAGHLRRLREMLEEATSHSLVLIDELGSGTDPEEGAALS | |
| | QAILEALLERGVKGMVTTHLSPLKAFAQGREGIQNASMRFDLEALRPTYE | |
| | LVLGVPGRSYALAIARRLALPEEVLKRAEALLPEGGRLEALLERLEAERLALE | |
| | AERERLRRELSQVERLRKALAEREARFEEERAERLKALEEEVRAELLKVEAE | |
| | LKALKEKARTEGKRDALRELMALRERYAKKAPPPPPPPGLAPGVLVEVPSL | |
| | GKRGRVVELRGEEVLVQVGPLKMSLKPQEVKPLPEAEPGKPLLAKPRREV | |
| | KEVDLRGLTVAEALLEVDQALEEARALGLSTLRLLHGKGTGALRQAIREAL | |
| | RRDKRVESFADAPPGEGGHGVTVVALRP | |
| PfuEndoMS | MEMTKAIVKENPRIEEIKELLEVAESREGLLTIFARCTVYYEGRAKSELGEG | 4 |
| Pyrococcus furiosus | DRIIIIKPDGSFLIHQKKKREPVNWQPPGSKVKMEGNSLISIRRNPKETLKV | |
| Cleavage | DIIEAYAAVLFMAEDYEELTLTGSEAEMAELIFQNPNVIEEGFKPMFREKPI | |
| | KHGIVDVLGVDREGNIVVLELKRRRADLHAVSQLKRYVDALKEEHGNKVR | |
| | GILVAPSLTEGAKKLLEKLGLEFRKLEPPKKGKKKSSKQKTLDFLNDTVRITG | |
| | ASPPEAIQ | |
| TkoEndoMS | MSKDKVTVITSPSTEELVSLVNSALLEEAMLTIFARCKVHYDGRAKSELGS | 3 |
| Thermococcus | GDRVIIVKPDGSFLIHQSKKREPVNWQPPGSRVRLELRENPVLVSIRRKPR | |
| kodakarensis | ETLEVELEEVYMVSVFRAEDYEELALTGSEAEMAELIFENPEVIEPGFKPLF | |
| Cleavage | REKAIGTGIVDVLGRDSDGNIVVLELKRRRAELHAVRQLKSYVEILREEYGD | |
| | KVRGILVAPSLTSGAKRLLEKEGLEFRKLEPPKRDSKKKGRQKTLF | |
| Ape Hjres | MDMPRRGVGYERELAKILWERGWAVIRGPASGGGSRSRVQPDLVAVR | 43 |
| Aeropyrum pernix | GGVVLVFEIKKARGETVYLDPGQVLGLLEWARRAGGDAWIALRLVGKG | |
| Cleavage | WRFHRADSLEHTRRGGFKISRPGGGLKLRDLLTLYGGGVRRIDSYLEG | |
| ApeDUF2122 | MPVEVIPVLHNVSSVQRVVDMARLSYSLGLDTLVVTKAYGGAAQSGVPE | 44 |
| Aeropyrum pernix | AMRLALKLGKSLVVLPELRDAVNLLSPTHVLAVTPSRAERLVGPGGLEGLE | |
| Cleavage | GRVLVVFSGGEPELDPSEAAGAIRVYIEGVEGKVGPIAEAALILYFLLRGGG | |
| | DGRG | |
| Ape EndoMS | MSQEASVDGRFGVASSPTIEEAAGLLEKLLDGSSMVVVAGVCSSEYEGRG | 45 |
| Aeropyrum pernix | ASVSTEGDKLLIVKPGGAVILHGPRGFRPLNWQPSTSHTEVATADGLLTLK | |
| Cleavage | FYRRTPREVLKIACGSIWYIAWVRFPEEGAFWMYMTEDDLRKAVALHPR | |
| | ELLGEDIRFFAEEKRTPSGKADLYGVDERGNIVIVEVKRVRADESAVRQLE | |
| | GYVRDYPTQAKVRGILVAPDISDAARRLLESRGLEFRRVDLKKAYSLLKPGR | |
| | GRSVLDFL | |
| ApeRecB | MPIEDGGVEVGEIDIVAEKGGSRYSVEVKAGMADINAVRQAYVNSLISG | 46 |
| Aeropyrum pernix | MKPMIVARGADEAARKLAEKLGVELIVLPDVVVSSTDDLKEIVEEAVDQAI | |
| Cleavage | LSLLEPLSHCENLEPEDLEVLEAIAKAKSFREAASKLGTAVEELAARVDRLKR | |
| | AGVLPRSSYKRMRAAAIIVLAGCRLLAGNRG | |
| AprHjres | MLLKILFEEWKQKYEKEIKRDAIEKSKAVITGKVTEHLIPFFPGFKYNPKDVR | 47 |
| Archaeoglobus | FIGSPVDLIVFDGLDEGDLRKIVFVEVKYGKSALSKRERLIRDAVTQGRVE | |
| profundus | WEVLRLET | |
| Cleavage | | |
| CmaHjres | MPLGRRGVNYERELANWLWSLGFAVIRAPASGGGVRRRFAPDLVAMFK | 48 |
| Caldivirga | GRVIILEVKYRGKPTPVSIRCDKVNRLIEFAGRAGGEAYVVVKYAREPWRI | |
| maquilingensis | MPIKPCNGDAAITYTTSEIKEASRLEDLVRSIININLINLL | |
| Cleavage | | |
| CsayqgF | MRILCLDIGSRRIGVAISDPLKIAAQPFCVLDLQKEDLYSCLDKIFNEHQIEKI | 49 |
| Caldicellulosiruptor | VIGYPVSKFHPDKATEKLQKIDEICSELERRYKVGIIKWDERFSTKAVERILR | |
| saccharolyticus | EENVSWQKRKKVVDKLAAVYILQGYLDFINSSRRLGLAPINSTPEKVEEILK | |
| Cleavage | TLIPVEEWIYVNHAMVDHGKSICRPIKPKCELCPLNELCPKIGV | |
| DkaHjres | MSNRSRGFSHERDLVRRLWDHGFAVIRAPASGSRARHVKYPDIVAIYHG | 50 |
| Desulfurococcus | KIVAMEVKTIKEERTIYVRREQVEKLQEFSRRAGATPFIAVKFVGSGEWFLI | |
| kamchatkensis | PLEKLAGSEGGTFKIPVETVKNSLRLKALISMIKGDKSILDYTMR | |
| Cleavage | | |
| DkaRecB | MSISSSRKWRSSELIALEYLEKQGFRIEETRKKIKIEGVEIGEVDAIAISPGGE | 51 |
| Desulfurococcus | KYAVEIKAGRIDVAGIRQAYVNALLLGLKPLIVAKGYADDSAMMLARELD | |
| kamchatkensis | VKVVELGDQYLVDSEELEIIVESAIYGLLRKILGVVTSKEPIPPQDYTVIEALA | |
| Cleavage | GSRDIKEFADSLKTTAENAMRQVRRIQSKGILPEDTKSYYELKMYAQIILLR | |
| | ERIRGLERLLASNCREKTQPSYP | |
| DmuEndoMS | MAPVNEPLILEKPDPAALPDLIRRAIGERNTVIVIGECSVEYEGRSTSRLGK | 6 |
| Desulfurococcus | GDRVLMIKQDGSVLVHRPTGYSPVNWQPDTSVIEAWINERGELSILAVRS | |
| mucosus | KPREVLNISFSRVDSVIIARLRDDAEFTMYLDEAEMKRVLVGNPELIEPGFR | |
| Cleavage | VVEDEKRLGSGQADIYGVDKEGRPVIVELKRITASRDAVLQLYGYVKAYEA | |
| | TYGKRPRGILIAPSFSPSAIETLLRLQLEYRQVDLRELYRLAVEKGLRRSLLEY | |
| | GRRPGERERGTGEKTRHE | |
| DtuHjres | MRVLAIDWGEKYIGLAISDPLRIIAQGLDVWEIKDEEDFVNRLKKLIKEYNV | 52 |
| Dictyoglomus turgidum | SEIVLGYPISLRGHENEKTKKIEYVAERIKTVVNLPIKFVDERFTTMEAERVL | |
| Cleavage | LEGDIKRRDRKLLKNKQAAVIILQKYLDSLSLDTKI | |
| DtuRuvC | MVVIGFDPGTAITGYGILNKGEDGISIIEYGALTTPSGWGIGRRLNYLFDQ | 53 |
| Dictyoglomus turgidum | VSSLLDLYNPDVVVMENIFFNKNIKTAINIGQAQGVIILAAEQHQKEISILTP | |
| Cleavage | LEVKLSVVGYGRATKNQIQYMVKEILKLKDIPKPDDVADALALCISYIYKQE | |
| | GC | |
| HbuHjres | MGRAQIRSKGFNAERELARKLWSRGFAVVRAPASGSKAKHVFYPDLVA | 54 |
| Hyperthermus butylicus | MYRGKIFVFEVKYRTTSETIYIEKEKILKLVDFAERAGGKAYIAIKLLGKGWL | |
| Cleavage | VVPVDNLAETAGGRYKIDLNSMKVLTLDEFVNRIQNESLTRYVARHV | |
| IagHhH | MSMKKTCVWNREDIVNIFEQIKIYYRDSIKIDDFVALKLVAEGAEPFEVLV | 55 |
| Ignisphaera aggregans | GIILSQNTSDRNAYRALLRLKNVLDDVITPDRILSIDPSVIINAINVAGLANR | |
| Cleavage | RLQSLLELSRHIKENPKFFNDLKNLSVDDARKALLSIYGIGYKTADVFLLMIY | |
| | KKPTFPIDTHIMRVLKRLGIVHEDMGYEDIRKFILGVVEHNPEELLSLHISLI | |
| | AHGRMICKARNPRCSECPINTKCCRIGVQ | |
| IagHjres | MAGNSIKGRRRGFHAERELVQKLWKMGFAVIRGPASGAKIKSGIYPDVV | 56 |
| Ignisphaera aggregans | AIKDGKIFVFEVKERKDIASIYVDKRQVEKIKEFAIRAGGEALIAVKIASVKS | |
| Cleavage | WKVINIDSLVDFNGSKFKIPKDAIESAEDLFNYLTKKITKTLDNYVIR | |
| IagEndoMS | MICRDVATSNCEDIKSTLESAKKRNVILIVGRTTIEYIGRAASESILADRLVIIK | 57 |
| Ignisphaera aggregans | PDGSLLIHEATKVEPFNWQPPRSIINFECINDKLRLKSIRLNPHEEVLVDFD | |
| Cleavage | YIDFIKICNISTTKLRIIGRESDVVSMIMNNVALIDKDSSIIGIDIPTPYGKIDIL | |
| | LKRSDGTFIVVEVKNEKAGVPAVIQLKRYVEFYKSKGYNVIGILIANDITDD | |
| | AYSILMKEGFKFVNLSSIITKPRDMGKLDKFLKT | |
| IhoHjres | MRSRVSFERELAGKLYQMGWAVMRAPASGAAAKRYLYPDLVALKKGRA | 58 |
| Ignicoccus hospitalis | VAIEVKTTKDKKYIYLERRQYEILKEWEERGGADAWVAVKVLYSGWSFYP | |
| Cleavage | LSSLKEAGKSFRLDLEGGLSLESFDALYSVKYTLTEFAKGIVEV | |
| Mja EndoMS | MMRLEKVFYLTNPTTKDLENFIDMYVFKYILILLARCKVFYEGRAKSQLEEG | 7 |
| Methanocaldococcus | DRVIIIKPDGAFLIHKDKKREPVNWQPSGSSIIWEVEDNFFILKSIRRKPKEE | |
| jannaschii | LKVVISEVYHACAFNCEDYEEINLRGSESEMAEMIFRNPDLIEEGFKPISRE | |
| Cleavage | YQIPTGIVDILGKDKENKWVILELKRRRADLQAVSQLKRYVEYFKNKYGED | |
| | KVRGILVSPSLTTGAEKLLKEENLEFKRLNPPKGSKRDLKHNIKTKKTTVLDE | |
| | WL | |
| MkaDUF2122 | MIPVTVVYHNVHSPRKVEEMARTVAGFGAKRFVISRALGSAAQEGVPKA | 59 |
| Methanopyrus kandleri | QRICLEAGVELLFFQDLEEALKALSPDVTYMAEDAEHGGAKPLDFDAVVE | |
| Cleavage | EIEQEREVCFVFGSARPGLTKQELELGDEAVYVGTERNVGEVGAVAILLHE | |
| | LRKRLNQ | |
| Mka NucS | MTKVLVEPDPEEVKQLSEALGRQPVILAGICEAEYRGRAESVAGPALRIA | 8 |
| Methanopyrus kandleri | MCKPDGTFILHNAMEKREPTNWNPAPSRQSIEVRDGCVVLRSRRLDVPE | |
| Cleavage | EVVVYFHKVLLACSLPKEGAKSEDSVFSLFRSEEDMKRVIREDPSVIEPGFR | |
| | PVGEEVECGAGVADVVGYDEEGRFVVLELKRTRAGVSAASQLRRYVEAF | |
| | REERGEEVRGILVAPSVTDRCRRLLEKYGLEWKKLEPVPLRDDGGKKQCTL | |
| | TEFLAGEGD | |
| MkaRecB | MLRRGKSAEEIAASILRKEGFEVVARNYRVELEDELVAEIDIVAEKDGERYA | 60 |
| Methanopyrus kandleri | VEVKAGTVGVDAVRQAYVAAKLTGYRPLVMGRRVHPSAEALADHLGVE | |
| Cleavage | VREFSEFVEVEPVDELAAVVSDLLQDKMLVLLSAASNVSWEDLYAALQG | |
| | DLRRVTELVRVRDRSQAEELIEALAFLKLMASETVGAVKGVEGDEFLLELD | |
| | PRVEVPDGTTLVVLNRWGSPAFVLAEPVSVGTHRARMRSWEGKVETGE | |
| | RVVYLGEVEG | |
| Mth NucS | MNCMKCKVSENPSIKEAYRLIEDGIRKRALVVILACCSASYEGRARSRLDA | 61 |
| Methanothermobacter | GERLIVIKPDGTFMVHQDRKVDPVNWQPPKSRSRAYIKRGSLYLESIRRD | |
| thermautotrophicus | PEERLEVEIHEAHLVSYYLARDVHDLMVAGHENDMGDMIIMHPHLIEKG | |
| Cleavage | FRPVAREYAVTSGFIDILGKDENGSLMIIELKSRKAGVSAVKQLKRYVDEFR | |
| | EDRVGVRGVLVAPSITHDAMEMLEEEGLEFREIEPPRELRSNRGVTLDNFL | |
| NeqHjres | MKKISSNVEREIQSLIWEKGFACLRIAGSGSSRYPAADLIAKIKDLYIIEVKYT | 62 |
| Nanoarchaeum equitans | HKDYVYIKDEQLEQIELLCKKFEAKPLIVIKFSRKGIYCTTNLRNKYTINDGDL | |
| Cleavage | VNFKEWLSAAPGFFTNQ | |
| PabCOG3372 | MLPKELLDAKRSRGKIQLNFANEEHLRLAKAVIIAFKSSLGQRYSELQEKLR | 63 |
| Pyrococcus abyssi | HLETASNYKKVRGFAKIIERECEFQVATSLDPLSVRRFLFERGYVTSELERIK | |
| Cleavage | VLSEAAQEFNTSIEEIERAVFADREEERVLVKIPEISEEELIKRYNLSLLQTLAF | |
| | NAVRLTFRVSSNHKRILRAIKRLGLMYEIQGDKIEITGPATLLKLTRKYGTSI | |
| | AKVIPEIIRAKEWWIRLELVEGKRLYIFELSSEDDVELPELEKIEEYSSSLEREF | |
| | SAKIKRILGVEVIYEPGIIKVGESAYIPDFLIRKGDKEVYVEIVGFWTKDYLRR | |
| | KLEKVTKLNIPLLLIVNDELFAEKAMRIKGKDVILMKKGKIPYKQVIMKLKE | |
| | MLTKS | |
| PabNucS | MRKVIIKENPSEEEIKELLDLAEKHGGVVTIFARCKVHYEGRAKSELGEGDR | 9 |
| Pyrococcus abyssi | IIIIKPDGSFLIHQNKKREPVNWQPPGSKVTFKENSIISIRRRPYERLEVEIIEP | |
| Cleavage | YSLVVFLAEDYEELALTGSEAEMANLIFENPRVIEEGFKPIYREKPIRHGIVD | |
| | VMGVDKDGNIVVLELKRRKADLHAVSQLKRYVDSLKEEYGENVRGILVAP | |
| | SLTEGAKKLLEKEGLEFRKLEPPKKGNEKRSKQKTLDFFTP | |
| PaeHjres | MSVKRKGSAKERELANFFWERGCAVLRGCSSGGGVRKRYVPDVVAICKG | 64 |
| Pyrobaculum aerophilum | KVLVFEVKYRSKYTPIKIEREKLEKLAVFAKRAGGEAYLLVKYGRNPWKVLE | |
| Cleavage | IRDKIDREEYEKAVELRAFLEALFSQTIDRYF | |
| PaeCOG3372 | MIPIDYLRASRKGREVRPRYLNDDKIASEVINMAKSAKTLGEFRNAVELISS | 65 |
| Pyrobaculum aerophilum | DKKLVRGLAHVLEQLIEIEKIDSKLVTRTRLEVFKAASALGYPLTEEERERVF | |
| Cleavage | QTVAARLKMGVNEVKALFLKAYEENRLIVKAPDIQPKQLVEMYNLALIQA | |
| | LLFKSLYVKALLPNAPALLKGLIRAVKGLGLMYIVEINGGQLEFRFDGPVSA | |
| | LRQTERYGTRLAKLVPYITSAEKWEIEAQVKLGERIYVFKESGRTAPPLPKT | |
| | PPHAEQFDSLVEQEFYKQVSKICHVEREPEALVVDGRVYIPDFKIGDLYVEI | |
| | VGFWTPDYLKRKYEKITKVGKPLLVLVSEELAMATWKQLMPNVVVFKDR | |
| | PRLSDVFKYIKPYCVNHR | |
| PaeNucS | MQQAVGMYVEAPTPDEAARLINSGRKLGIVIAVGVCEVFYTGRAAASLG | 66 |
| Pyrobaculum aerophilum | PTRRLVILKRDGTLLVHEAEKAQPKIWNPPGSSTAAYVEGGKLVVKSIRSR | |
| Cleavage | PFETVRVIFDKVDFIAAFDVGATELTLVGSERDVVGALVKNPSIIEEGLEVV | |
| | GVEVPTDVGHLDILARDKNGRYVIIEVKRDLATHEAVFQLARYVELYRRKG | |
| | YDVRGILVAGDITASAYDYLKRYGLNFVKINPRELMVLTNKNPLNAK | |
| PaeRecB | MGARFERFVLDLLPAIGLIPKATRFKVYREGVEVGEVDIVAVDEKGETYAIE | 67 |
| Pyrobaculum aerophilum | VKAGRVDISGIRQAYTNAKILGARPMVIARGYADEGARQLARELGVEVILL | |
| Cleavage | PDYFFLSIDDLYVAFTNALARFVTAVVSVYTNLSENEIEAIRECKDMQCIAK | |
| | KSIARLYSKGSPKRLKTWRLYY | |
| PhoCOG3372 | MLPRELLDARRSRGRIYLNFASDEHLKLAKAVLIAFKSSIGQSYAELQEKLR | 68 |
| Pyrococcus horikoshii | HLETASNYKKVRGFAKIIERECKFSMATNLDPWEVRKFLFERGYVTSEFDRI | |
| Cleavage | KVLREAAEHFGASIEEIERAIFADREEEKILEEVPEINPEELIKRYNLSLLQTL | |
| | MFEAVRLTFKVSSNYKEILRGVKKLGLMYEVIDEGIEVTGPASLLKLTRKYG | |
| | TSIAKLIPGIVKAEEWWMRAEVVEDKRIYIFELSSEESVMLPKIMERIEYSSS | |
| | LEREFASKIKRILGVDVIYEPEIIKVGNYAYIPDFLIKKGNKKVYVEIVGFWTR | |
| | DYLKRKLEKISKAKIQMLLIVNDELFAEKASRFSGKEVLLMKKGKIPYKDVI | |
| | MKIKEMLKS | |
| PhoNucS | MKKVITRENPTVEEVKELLDIAEKHGGVVTIFARCRVYYEGRAKSELGEGD | 10 |
| Pyrococcus horikoshii | RIVIIKPDGSFLIHQNKKREPVNWQPPGSKVSMRENSIISIRRKPHERLEVE | |
| Cleavage | LMEVYAVTVFLAEDYEELALTGSEAEMAKLIFENPNVIEEGFKPMFREKQI | |
| | KHGIVDIMGLDKDGNIVVLELKRRKADLHAVSQLKRYVDSLKEEYGEKVR | |
| | GILVAPSLTEGAKKLLEKEGLEFRKLEPPKNNDNKREVKQKTLDFFTPC | |
| PisEndoMS | MFVQNPSIDEAVRIINSGRKRGVVVVVGICEVVYSGRAAATLKPGRRLVIV | 11 |
| Pyrobaculum | KRDGTLLVHEAEKAQPKIWNPPGSSTAAYVEGGRLVIKSVRSRPFESVRVY | |
| islandicum | FSSLDFVAAFDVETSELELVGSEKDVVEALVKAPWLIEEGLEVVGVEVPTD | |
| Cleavage | VGHIDILARDREGRHVVVEVKRDVATHDAVFQLARYVELYRKRGERARGI | |
| | LVASDITAAALEYLRRYGLEFVKVNPRELMASINKKVG | |
| PyfHhH | MPEGGTTRDIIESVLEKPSVFKSREKLDPDYVPEKLPHREQQLKQLAMYFR | 69 |
| Pyrolobus fumarii | GIITDPGSTSHRALIVGPIGVGKTASARRFTMDFAEIARKRGLTLRMVYINC | |
| Cleavage | HAVRTLYSVVTSIASQLEIPMPTRGYSAREVFERVLEKLEERDEHAIIILDEF | |
| | DYFIETSGNDAVYFLVRVYDEYPMFKRRMHYIFIMRDLKHLNMLNPATV | |
| | NYLLKNVVTFQPYTVAQLYDILQYRAELSFYPGTVGDEVIRYIAELTGIDGH | |
| | GEGNARQALQILTLAGEYADNEGSDRITIEHVRKAHALVNPHAVRIQDIIL | |
| | EGSLTLHQLLLLLAIIRVLRSKETEPYAKMGEVEQEYRAVCEEFGEKPRGHT | |
| | QVYEYIRDLKLKGVIDAKPSGKGVRGRTTLIGLSAAPLDILEKTVIDAIRKFK | |
| | AGDVP | |
| PyfHjres | MERVTTVYYYTPQQEVYVPDTSVLIEGIVSKLIREGRIRGKIVIHRAVIAELE | 70 |
| Pyrolobus fumarii | HQANLGKTIGFAGLREIREIRRLAEEGLIDFEIGGNRPSPSEIAHAKKGAIDA | |
| Cleavage | LIRDYALEIGATLITGDRIQALVAEAMGIPVIYIPPREGERLTIERLFDEQTM | |
| | SVHLKEGVVPRAKKGKPGSWKLVDLDDRPMTRDELEEIAREIVEAAKRRK | |
| | DGFVEIDRAGSTIVQLGDYRVVITRPPLSEAWEITIVRPVARLSLEDYNLPP | |
| | KLLRRLEERAEGILIAGAPGAGKTTFAQALAEYYARKGKIVKTIESPRDMRL | |
| | PPEITQYSKNYASIGELHDILLLSRPDYTVFDELRTDEDFKLYIDLRLAGIGMI | |
| | GVVHATTPIDAIQRFLQRVDIGLLPSIIDTVIFIDKGQIEAVYELRMTVKLPT | |
| | GLREAELARPVVEVVDFLTGELVYEIYTFGEQRVVVPVKKVKMGALEERV | |
| | KKLVERLIPGADVEISDEGVVIVNIPRIAAKTVMKKIRKLKKLEEKYGVHIRV | |
| | NLVG | |
| SacHjres1 | MSNKTKGSTLERYLVSRLRDKGFAVIRAPASGSNRKDHVPDVIAMKSGVII | 71 |
| Sulfolobus | LIEMKSRKNGNKIYIQKEQAEGIKEFAKKSGGELFLGAKIAKDLKFLRFDELR | |
| acidocaldarius | RTEAGNYVADLETIKSGMDFDELVRYVEGKISKTLDSFM | |
| Cleavage | | |
| SacHjres2 | MNRDIGKSAERELVSILRSEGFNAVRIPTSNSSPNPLPDIFATKGNILLAIEC | 72 |
| Sulfolobus | KSTWEHKVKVKDRQVIKLFEFLSMFTMEGRALIAVKFKEIHKWKVMEIREI | |
| acidocaldarius | KEVEVTVDNSIFLENYISQFLENYIPQAGRELSKL | |
| Cleavage | | |
| SacEndoMS | MFKVLLEPDLQEALIFLNESVNALLTIYSECEILYSGRAKSRASLSPRLTIIKPD | 12 |
| Sulfolobus | GSVIIHGPTKREPVNWQPPGSRIEYSIESGVLTVNAERKRPKERLSILHHRV | |
| acidocaldarius | YYITSSEVKPGEFFLVGREKDEVDFIINNPDVIEGGFKPIHREYRTPYGTVDL | |
| Cleavage | IGKDKEGNLVVLEFKRAKASLQAVSQLYRYIMYFKEIGENARGILVAPGISE | |
| | NALNLLKRLELEYVNISDKLGDSTISRPINYVPNLQRD | |
| SheHjres | MPNLNRRRGFAHERDLLLKLWRRGFAVIRAPASGAKARRFAVPDIVAIKN | 73 |
| Staphylothermus | NRVLAFEVKTAEKKKTIYIPKHQVEKLLVFIRRAGGYGFIAVKIVGESGWRFI | |
| hellenicus | EVDKLEKTASGNYKVSPDMLVKSFKLGDLVSFVQGNKRIDEYI | |
| Cleavage | | |
| She EndoMS | MKYSKNELLMVKLIKTYLNPTLSEALEIISKAVSNKELTIIVGECSVDYQGRS | 74 |
| Staphylothermus | ESRLTPGERVIIIKQDGAFLVHRPTGYSPVNWQPTTSIIETRLDRDKLIIMA | |
| hellenicus | VRRKPRETIWVYLMRIYAIITGKLIDNGEFIMYMDEHEIRDILYEHPELIEDG | |
| Cleavage | LKIMEKEKKIGEGYADLFGVDARKTPVIIEIKRVTATREAILQLYNYVQTYQ | |
| | RQTGVKPRGILVAPTITSSAIESAYKLGLEWKEINLQKIWKYKKDRNRKHG | |
| | TLFDFFKNEK | |
| SmaHjres | MPNLNRRRGFAHERDLLLKLWRRGFAVIRAPASGAKARRFAVPDIVAIKN | 75 |
| Staphylothermus | NIVLAFEVKTAEKKKTIYIPKHQVKKLLVFVKRAGGYGFIAIKIIGESGWRFIE | |
| marinus | VNKLEKTASGNFKVSPNMLVKSFKLGDLVSFVQGNKKIDEYI | |
| Cleavage | | |
| SsoCOG3372 | MLTSDLARFKIENQRIIPLFATDSDIDVAKEVIDMFKIGAKVGDILEDLKYLS | 76 |
| Sulfolobus solfataricus | KIYDYKLVKGLGKIYLRYCIVESATKIDYIELRRQLFSRGPVLEENDKERVLKE | |
| Cleavage | VGDLFHVDPIKAMYEDLDVEKKIVELPKFSPEDLLKIYNLSLLQTIIFNAYKV | |
| | TVSVSDGWKEIARRIKMLGLMYLAYENPLRIEIFGPLSLVKMTEKYGRNLA | |
| | ALVPFLVSKNKWTIIADIVLGKNKRRTYRLELSSGYSKLFKYINDEEMEKRF | |
| | DSSIEEKFYEEFRRVIRDWNIVREPEPIVIEKRLYFPDFVLSKGNIKVYVEIM | |
| | GFWTKEYVNSKIEKLRNFKYPILVLLNEELSYENYIPDTLNVIKFKKKIDIGKL | |
| | YSALRGFQANVNEDIDLSDVNDDIILIKELSAKYNVNEKIVRSKLMQRPDYI | |
| | VLKNYAIKKTFIEELKKEDFSNTQLSSLVNKYGNYIVDIIDYLGYKIVWKNIS | |
| | DAIVEKIKEV | |
| SsoDUF2122 | MKELYFGLHNLTSNQRLLDFSKLAFNVKYVKYLVLTKVGGTAAQSGVPEV | 77 |
| Sulfolobus solfataricus | NKLAFKYNKPILVLPELKDAIELLKPEITLLVSQNAEKQIDFNDMIKHDKILIV | |
| Cleavage | FSGLEGGGFNKIEQSLGEYVRILEDTQDLGAVSLASFFLCKYLQLVEGKV | |
| SsoNucS | MYSVLLNPSNTEIYSFLTDRIYRELIVIFATCKVNYKGRAESVASESPRLIILKP | 78 |
| Sulfolobus solfataricus | DGTVIIHESVKREPLNWQPPGTKIEIMNDYPLKIVAQRNRPKEVIEIDLKEV | |
| Cleavage | FYITSAEVKEGDFTIKGREIDIVNTIIQNPSMIEEGFVPLTREYNTPYGKIDLI | |
| | GLDKKGNFVIIEVKRSKAQLNAISQLYRYYLHMREIKGDKIRGILVAPDLTIH | |
| | ARELLQKLGLEFIRYDIKNYSS | |
| TacCOG3372 | MFPAELMVVRKAQDGTIRPLMIAPDHVDIAEKVIEIYRSSAGRTRKEISKDI | 79 |
| Thermoplasma | VTIEYGIKNPKIVRGLALIMDRMSTFRNRSRVDSKQLRGFLFQMGPAVSP | |
| acidophilum | EERQEKIALAAEHFSVSPQDIEEAIYGDMDSENVLDQCPSITPEHLNRRYN | |
| Cleavage | LEQLTTLMYRSKFIEVSGINNWYRFISLIKRQGLVFEAQGNPLMSVRIDGP | |
| | NSVFNNMERYGSSMARVVERLTAFPGWKLHAEVEIKDRVYSVDLDSSISY | |
| | YLPETDIEEEEAIPDPVVIGTRVFFPTRIINVHGQDVYVDIVYHMSPEAIKRR | |
| | DEMIRSSGIKWITAVVGECKKFQGVLCFRNRVDWDAIIAKASEEYPATTD | |
| | ALREEIDRLYPNTEAILDLLDSRSIPLSHLEKIGYRIKWNGILPEIVRST | |
| TagHjres | MSNRRRGFSHERDLAQKLWNHGFAVIRSPASGSKAKNILYPDIVAIYHGR | 80 |
| Thermosphaera | VLAIEAKTVRKERTIYLKEQQVEKLIEFSRRAGGEAFVAVKIVGTGEWRFVS | |
| aggregans | LNSLRETGGLKITKAHLCNSLKLEDVISIVKGVRKLDEFEKTE | |
| Cleavage | | |
| TagEndoMS | MATYRILENPDLESALKTIKEGLMRNHLLIIIGECYIDYEGRSASKLGLGERIV | 81 |
| Thermosphaera | IIKQDGAVLVHRPKGYSPVNWQPSTTTIEVWLRSGEGLSLLAVRNRPREYL | |
| aggregans | RILFTKIFTIIEGVFHDSAEFVMYLSESEIRDIIFENPDLLEPGFKPLEKEKRIG | |
| Cleavage | QGAVDIYGLDSNNNHVLVEIKRVMGDREAVLQLYNYVENYKNGVENNV | |
| | RGILLAPSFTPGALELLAKLKLEFKEIDLRKLRLLSQSSKKKPNSTLFEYMNEK | |
| | GRD | |
| Tba EndoMS | MKVEAKVEPSHEEIIEILDKALSVEAIITLFAYCRVFYEGRAKSELGPGDRVIII | 82 |
| Thermococcus | KPDGSFLIHQKNKREPVNWQPPGSVVSIVLEDGRIMLRSVRRKPKETLEV | |
| barophilis | ELIKTYLVSYFQAEDYEELTLTGSEAEMADLIFENPSLIEEGFKPLFKEKPIKH | |
| Cleavage | GIVDVLGKDKHGNLVVLELKRRRADLHAVSQLKRYVDSLREEHKNVRGIL | |
| | VAPSLTAGAKKLLEKEGLEFKKLNPPKREKRKKGKQKTLDFLSP | |
| TmaHjres | MGSLRILGVDPGYGIVGIGIIEVSGNRISHVFHGTIETPKNLPAEKRLKRIYE | 83 |
| Thermotoga maritima | EFLKVLERFSPDECAMEKLFFVKNVTTAIGVGEARGVLFLALAEKNIPVFEY | |
| Cleavage | APNEVKVSLSGYGRASKKQIQENVKRFLNLSEIPRPDDAADALAIAWCHA | |
| | LQSRARRVTHEKD | |
| TmaMrr | MIYIILSLLSFSLLVLFLQWGKKRRKQDLRNLLEAGLKNPYRFEEFAREYLKE | 84 |
| Thermotoga maritima | HGFRSVRTTRKSKDFGADIVAKRRGSTVVFQVKRRNSTVEKEVVKELVAA | |
| Cleavage | AYIYGATEVGIFTNGELSTGLKKELEELKRSGGFIKRVHVVKNINPEEL | |
| TpeHjres | MSREVVSRRQKAFNVERRLVKMLSSNRENYVFRVPVSGVGENFPDVFLV | 85 |
| Thermofilum pendens | NNVEDRVVAFEVKTTVNSKVKVKAHQVSKLFRFLEAFKKYKTREAVIAVW | |
| Cleavage | FSSEGKWVFRRVDGLFASDIVITSEDESDWKP | |
| Tpe EndoMS | MTVVEELRAAVSSKKLVVLVARCTVTYEGRASSKLEEGDRVILIKEDGSVIV | 86 |
| Thermofilum pendens | HRPVGYEPVNYQPPGSVVSVEGDDDGFRIIVTRTKRRERLLIEVHSVKHFF | |
| Cleavage | SAKLEDSARFTMWGDEEDLRRAILADPSKMLGENLKPVGAEVSLGSAGF | |
| | ADAVFVDSEGNLVVVEVKREKASVEAVYQLKRYVERIKRETGRKVRGILAA | |
| | PGFTVSAIRALKAEGLEYRQVSMKDAREVLSRLTAFW | |
| TteHjres | MGVKFETFVLDLLPSLGLTPLAHRYKVIKDGVELGEIDVLAEDSQKNLYAV | 87 |
| Thermoproteus tenax | EIKAGKVDVSGIRQAYINAKLVGAKPLIISRGYADESAKRLADELEVAVITLP | |
| Cleavage | DYVFLSIDELVNAMSLAFARSLAYLVAALMEMDDRVAEALSECPDYQCFC | |
| | SKVENCDQVLQRLSRRMPANYETYRNLAIIRKALRDHCRPP | |
| TthMutM | MPELPEVETTRRRLRPLVLGQTLRQVVHRDPARYRNTALAEGRRILEVDR | 88 |
| Thermus thermophilus | RGKFLLFALEGGVELVAHLGMTGGFRLEPTPHTRAALVLEGRTLYFHDPR | |
| Cleavage | RFGRLFGVRRGDYREIPLLLRLGPEPLSEAFAFPGFFRGLKESARPLKALLLD | |
| | QRLAAGVGNIYADEALFRARLSPFRPARSLTEEEARRLYRALREVLAEAVEL | |
| | GGSTLSDQSYRQPDGLPGGFQTRHAVYGREGLPCPACGRPVERRVVAG | |
| | RGTHFCPTCQGEGP | |
| Tth MutY | MEAWRKALLAWYRENARPLPWRGEKDPYRVLVSEVLLQQTRVEQALPY | 89 |
| Thermus thermophilus | YRRFLERFPTLKALAAASLEEVLRVWQGAGYYRRAEHLHRLARSVEELPPS | |
| Cleavage | FAELRGLPGLGPYTAAAVASIAFGERVAAVDGNVRRVLSRLFARESPKEKE | |
| | LFALAQGLLPEGVDPGVWNQALMELGATVCLPKRPRCGACPLGAFCRG | |
| | KEAPGRYPAPRKRRAKEERLVALVLLGRKGVHLERLEGRFQGLYGVPLFPP | |
| | EELPGREAAFGVRSRPLGEVRHALTHRRLRVEVRGALWEGEGEDPWKRP | |
| | LPKLMEKVLRKALPLLAHAGVVPLPDA | |
| TthRuvC | MVVAGIDPGITHLGLGVVAVEGKGALKARLLHGEVVKTSPQEPAKERVG | 90 |
| Thermus thermophilus | RIHARVLEVLHRFRPEAVAVEEQFFYRQNELAYKVGWALGAVLVAAFEA | |
| Cleavage | GVPVYAYGPMQVKQALAGHGHAAKEEVALMVRGILGLKEAPRPSHLAD | |
| | ALAIALTHAFYARMGTAKPL | |
| TuzEndoMS | MLRILEPEPREAAAFINTNRKRGLIAVFCICGGTYRGRAAADLPTGPYLILIK | 91 |
| Thermoproteus | PDGSLLVHGSEKATPLVWNPPGSSNMAVVEGGTLVLKSLRTRPSESVVLK | |
| uzoniensis | IERVFEIVLFDAGSSSVRLRGTEKDIVDMLVKNPDIIEPGLKVVGVEVPTEA | |
| Cleavage | GHIDILALDKNGEYIVVEVKRDVADHEAVFQLRRYVEAVAKARGRARGIL | |
| | VAADITSSAFHYLREYNLGFVKIRPRELAEKLLNKTEFEDTSIDVEK | |
| TvoCOG3372 | MFPLELLVAKKTENGSIRPIVIPPDSKWPAENVIDTFRSSIGLKRRELYERIK | 92 |
| Thermoplasma | DIEYQAKNPKTVRSMALLLERISKFRDSFQVPSSEVRRFLFSLGPAVRPEER | |
| volcanium | HTLVEMAAKHFSVGKQEIEDALYGDIEPEQRLISVPDITPDWLVRNYNLE | |
| Cleavage | QIETLIYKAKSMVVKKLSNWLPFIDAIKELGLMFEALNEEGLTVIIDGPNSIF | |
| | GGMDRYGSALAQAFSLLAGSSSWSISATVSIGGKDYSVQLDQSLNYYLPE | |
| | KREGMLDKTYPEPINIDGRLYFPSSVMDIDGKKVYVDIVRNDDINRILKRD | |
| | SKIRASGYNWITVYIGPSKNWSNYPLRFRSKIDWSLVQAYARKTLPKETKA | |
| | DYVMAALSKLYPDIDAIIDFLDSNGLPLSIVEKYGYKVSWNGIVPSIEKS | |

*The term “Binding” refers to mismatch binding activity (mismatch binding proteins). The term “Cleavage”
refers to mismatch cleavage activity (mismatch endonucleases).

### INCORPORATION BY REFERENCE

All publications, patents, and patent applications mentioned in this specification are herein incorporated by reference to the same extent as if each individual publication, patent, or patent application was specifically and individually indicated to be incorporated by reference. This includes following patent documents U.S. Patent Publication Nos. 2003/0152984; 2006/0115850; 2006/0127920; 2007/0231805; 2007/0292954; 2009/0275086; 2010/0062495; 2010/0216648; 2010/0291633; 2011/0124049; 2012/0053087; and 2017/253909. U.S. Pat. Nos. 5,580,759; 5,624,827; 5,869,644; 6,110,668; 6,495,318; 6,521,427; 7,704,690; 7,833,759; 7,838,210; 8,224,578; 10,626,383; and 10,196,618. PCT Publications WO 2005/095605; WO 2010/040531; WO 2011/102802; WO 2013/049227; WO 2016/094512; and WO 2020/001783.

Exemplary Subject Matter of the Invention is represented by the following clauses:

Clause 1. A method for generating an error corrected population of nucleic acid molecules, the method comprising:
(a) assembling oligonucleotides with regions of terminal sequence complementarity by primary assembly PCR to form a population of assembled nucleic acid molecules, and
(b) amplifying the population of assembled nucleic acid molecules formed in step (a) by primary amplification to form a population of amplified assembled nucleic acid molecules, and wherein steps (a) and/or (b) are performed in the presence of one or more thermostable mismatch recognition proteins.

Clause 2. The method of clause 1, wherein at least one of the one or more thermostable mismatch recognition proteins is a thermostable mismatch binding protein.

Clause 3. The method of clause 2, wherein the thermostable mismatch binding protein is selected from a mismatch binding protein having an amino acid sequence set out in Table 13 or Table 15.

Clause 4. The method of clause 1, wherein at least one of the one or more thermostable mismatch recognition proteins is a thermostable mismatch endonuclease.

Clause 5. The method of clauses 1 or 4, wherein the thermostable mismatch endonuclease is selected from an endonuclease having an amino acid sequence set out in Table 12 or Table 15.

Clause 6. The method of clauses 4 or 5, wherein the thermostable mismatch endonuclease is TkoEndoMS.

Clause 7. The method of any of clauses 1 to 6, wherein a high-fidelity DNA polymerase is used in steps (a) and/or (b).

Clause 8. The method of clause 7, wherein the high-fidelity DNA polymerase is a component of an error reducing polymerase reagent.

Clause 9. The method of clauses 7 or 8, wherein the high-fidelity DNA polymerase is a polymerase have an amino acid sequence selected from the group consisting of: (1) DNA Polymerase 1, (2) DNA Polymerase 2, (3) DNA Polymerase 3, (4) DNA Polymerase 4, (5) DNA Polymerase 5, (6) DNA Polymerase 6, (7) DNA Polymerase 7 set out in Table 14.

Clause 10. The method of clauses 8 or 9, wherein the error reducing polymerase reagent comprises one or more amine compounds.

Clause 11. The method of clause 10, wherein the one or more amine compounds are selected from the group consisting of:
(a) dimethylamine hydrochloride
(b) diisopropylamine hydrochloride,
(c) ethyl(methyl)amine hydrochloride, and
(d) trimethylamine hydrochloride.

Clause 12. The method of any of clauses 1 to 11, wherein at least one of the one or more thermostable mismatch recognition proteins is present in step (a).

Clause 13. The method of any of clauses 1 to 12, wherein at least one of the one or more thermostable mismatch recognition proteins is present in step (b).

Clause 14. The method of any of clauses 1 to 13, wherein one or more error correction steps are performed after primary amplification.

Clause 15. The method of any of clauses 1 to 14, wherein post-primary amplification of the population of amplified assembled nucleic acid molecules is performed after step (b).

Clause 16. The method of any of clauses 1 to 15, wherein the population of amplified assembled nucleic acid molecules are contacted with one or more mismatch recognition proteins prior to the post-primary amplification.

Clause 17. The method of clause 16, wherein at least one of the one or more mismatch recognition proteins is a mismatch endonuclease.

Clause 18. The method of clause 17, wherein the mismatch endonuclease is a non-thermostable mismatch endonuclease.

Clause 19. The method of clause 18, wherein the non-thermostable mismatch endonuclease is selected from the group consisting of:
(a) T7 endonuclease I,
(b) CEL II nuclease,
(c) CEL I nuclease, and
(d) T4 endonuclease VII.

Clause 20. The method of any of clauses 1 to 19, wherein the population of amplified assembled nucleic acid molecules comprises a subfragment of a larger nucleic acid molecule and are combined with another nucleic acid molecule that is also a subfragment of the larger nucleic acid molecule, to form a nucleic acid molecule pool.

Clause 21. The method of clause 20, wherein the nucleic acid molecules of the nucleic acid molecule pool are assembled by secondary assembly PCR to form the larger nucleic acid molecule.

Clause 22. The method of clause 21, wherein the subfragments are contacted with the one or more mismatch recognition proteins prior to or during assembly by secondary assembly PCR.

Clause 23. The method of any of clauses 20 to 22, wherein the larger nucleic acid molecule is heat denatured, then renatured, followed by contacting with the one or more mismatch recognition proteins.

Clause 24. The method of clause 23, wherein the at least one of the one or more mismatch recognition proteins is a mismatch binding protein.

Clause 25. The method of clause 24, wherein the mismatch binding protein is bound to a solid support.

Clause 26. The method of any of clauses 1 to 25, wherein the population of amplified assembled nucleic acid molecules are sequenced.

Clause 27. The method of any of clauses 1 to 26, wherein the population of amplified assembled nucleic acid molecules contains fewer than two errors per 1,000 base pairs.

Clause 28. A composition comprising a thermostable mismatch recognition protein, a DNA polymerase, and one or more amine compound.

Clause 29. The composition of clause 28, wherein the DNA polymerase is a high-fidelity DNA polymerase.

Clause 30. The composition of clause 29, wherein the high-fidelity DNA polymerase is a component of an error reducing polymerase reagent.

Clause 31. The composition of clauses 29 or 30, wherein the high-fidelity DNA polymerase comprises an amino acid sequence set out in Table 14.

Clause 32. The composition of clause 28, wherein the one or more amine compound is selected from the group consisting of:
(a) dimethylamine hydrochloride,
(b) diisopropylamine hydrochloride,
(c) ethyl(methyl)amine hydrochloride, and
(d) trimethylamine hydrochloride.

Clause 33. The composition of any of clauses 28 to 32, further comprising two or more nucleic acid molecules.

Clause 34. The composition of clause 33, wherein the two or more nucleic acid molecules are subfragments of a larger nucleic acid molecule.

Clause 35. The composition of any of clauses 33 to 34, wherein the two or more nucleic acid molecules are single-stranded.

Clause 36. The composition of clause 35, wherein the two or more single-stranded nucleic acid molecules are less than 100 nucleotides in length.

Clause 37. The composition of clause 35, wherein the two or more single-stranded nucleic acid molecules are from about 35 to about 90 nucleotides in length.

Clause 38. The composition of clause 35, wherein the two or more single-stranded nucleic acid molecules are from about 30 to about 65 nucleotides in length.

Clause 39. The composition of any of clauses 28 to 38, wherein the thermostable mismatch recognition protein is a mismatch endonuclease.

Clause 40. The composition of clause 39, wherein the thermostable mismatch endonuclease is selected from an endonuclease having an amino acid sequence set out in Table 12 or Table 15.

Clause 41. The composition of clause 40, wherein the thermostable mismatch endonuclease is TkoEndoMS.

Clause 42. The composition of any of clauses 28 to 38, wherein the thermostable mismatch recognition protein is a mismatch binding protein.

Clause 43. The composition of clause 42, wherein the thermostable mismatch binding protein is selected from a mismatch binding protein having an amino acid sequence set out in Table 13 or Table 15.

Clause 44. The composition of any of clauses 33 to 34, wherein at least one of the two or more nucleic acid molecules are single-stranded and at least one of the two or more nucleic acid molecules are double-stranded.

Clause 45. A method of generating a nucleic acid molecule with a predetermined sequence, the method comprising:
(a) providing a plurality of single-stranded oligonucleotides with complementary overlapping regions, each of the single-stranded oligonucleotides comprising a sequence region of the target nucleic acid molecule, wherein the plurality of single-stranded oligonucleotides comprises:
   (i) a plurality of internal oligonucleotides having overlapping sequence regions with two other oligonucleotides in the plurality, and
   (ii) two terminal oligonucleotides designed to be positioned at the 5′ and 3′ terminal ends of the full-length nucleic acid molecule and having an overlapping sequence region with one of the internal oligonucleotides in the plurality,
(b) assembling the plurality of oligonucleotides by primary assembly PCR to obtain assembled double-stranded nucleic acid assembly products,
(c) combining at least a portion of the assembly products obtained in step (b) with a pair of primers, wherein the primers are designed to bind to the 5′ and 3′ terminal ends of the assembly products and performing a PCR amplification reaction to produce amplified assembly products,

wherein step (b) and/or step (c) is conducted in the presence of one or more thermostable mismatch recognition protein.

Clause 46. The method of clause 45, further comprising (d) conducting one or more error correction steps, wherein an error correction step comprises:
(iii) denaturing and reannealing the amplified assembly products of step (c) to generate one or more mismatch containing double-stranded nucleic acids, and
(iv) treating the mismatch containing double-stranded nucleic acids with one or more mismatch recognition protein, and
(v) optionally, conducting an amplification reaction.

Clause 47. The method of clause 46, wherein the mismatch recognition protein used in step (d) is a mismatch endonuclease or a mismatch binding protein.

Clause 48. The method of clause 47, wherein the mismatch endonuclease is T7 endonuclease I.

Clause 49. The method of clause 47, wherein the mismatch binding protein is MutS.

Clause 50. The method of clauses 45 or 46, wherein the thermostable mismatch recognition protein is as thermostable mismatch endonuclease.

Clause 51. The method of clause 50, wherein the thermostable mismatch endonuclease is derived from hyperthermophilic Archaea, optionally wherein the hyperthermophilic archaeon is Pyrococcus furiosus or Pyrococcus abyssi.

Clause 52. The method of any of clauses 45 or 46, wherein the thermostable mismatch recognition protein is selected from the group of proteins having an amino acid sequence set out in Table 12, 13, or 15, and variants thereof having at least 95% sequence identity thereto.

Clause 53. The method of any of clauses 49 to 52, wherein the thermostable mismatch recognition protein is obtained by in vitro transcription/translation.

Clause 54. The method of any one of clauses 45 to 53 wherein one or more of steps (b), (c) and (d) (iii) is conducted in the presence of a high fidelity DNA polymerase, optionally wherein the polymerase is selected from the group consisting of PHUSION™ DNA polymerase, PLATINUM™ SUPERFI™ II DNA polymerase, Q5 DNA Polymerase, and PRIMESTAR GXL DNA Polymerase.

Clause 55. The method of any one of clauses 45 to 53 wherein one or more of steps (b), (c) and (d) (iii) is conducted in the presence of a high fidelity DNA polymerase, optionally wherein the polymerase is a polymerase having an amino acid sequence selected from the group consisting of: (1) DNA Polymerase 1, (2) DNA Polymerase 2, (3) DNA Polymerase 3, (4) DNA Polymerase 4, (5) DNA Polymerase 5, (6) DNA Polymerase 6, (7) DNA Polymerase 7 set out in Table 14.

Clause 56. The method of any one of clauses 45 to 53, wherein two or more amplified assembly products are pooled prior to conducting the one or more error correction steps.

Clause 57. The method of any one of clauses 46 to 53, further comprising treating the amplified assembly products with an exonuclease prior to the one or more error correction steps, optionally wherein the exonuclease is Exonuclease I.

1. A method for generating an error corrected population of nucleic acid molecules, the method comprising:
(a) assembling oligonucleotides with regions of terminal sequence complementarity by primary assembly PCR to form a population of assembled nucleic acid molecules, and
(b) amplifying the population of assembled nucleic acid molecules formed in step (a) by primary amplification to form a population of amplified assembled nucleic acid molecules, and
wherein steps (a) and/or (b) are performed in the presence of one or more thermostable mismatch recognition proteins.

2. The method of claim 1, wherein at least one of the one or more thermostable mismatch recognition proteins is a thermostable mismatch binding protein.

3. (canceled)

4. The method of claim 1, wherein at least one of the one or more thermostable mismatch recognition proteins is a thermostable mismatch endonuclease.

5. (canceled)

6. The method of claim 4, wherein the thermostable mismatch endonuclease is TkoEndoMS.

7. The method of claim 1, wherein a high-fidelity DNA polymerase is used in steps (a) and/or (b).

8. The method of claim 7, wherein the high-fidelity DNA polymerase is a component of an error reducing polymerase reagent.

9. (canceled)

10. The method of claim 8, wherein the error reducing polymerase reagent comprises one or more amine compounds.

11. (canceled)

12. The method of claim 1, wherein at least one of the one or more thermostable mismatch recognition proteins is present in step (a) or in step (b).

13. (canceled)

14. The method of claim 1, wherein one or more error correction steps are performed after primary amplification.

15. The method of claim 1, wherein post-primary amplification of the population of amplified assembled nucleic acid molecules is performed after step (b).

16.-19. (canceled)

20. The method of claim 1, wherein the population of amplified assembled nucleic acid molecules comprises a subfragment of a larger nucleic acid molecule and are combined with another nucleic acid molecule that is also a subfragment of the larger nucleic acid molecule, to form a nucleic acid molecule pool.

21.-27. (canceled)

28. A composition comprising a thermostable mismatch recognition protein, a DNA polymerase, and one or more amine compound.

29. The composition of claim 28, wherein the DNA polymerase is a high-fidelity DNA polymerase.

30.-32. (canceled)

33. The composition of claim 28, further comprising two or more nucleic acid molecules.

34. The composition of claim 33, wherein the two or more nucleic acid molecules are subfragments of a larger nucleic acid molecule.

35.-44. (canceled)

45. A method of generating a nucleic acid molecule with a predetermined sequence, the method comprising:
(a) providing a plurality of single-stranded oligonucleotides with complementary overlapping regions, each of the single-stranded oligonucleotides comprising a sequence region of the target nucleic acid molecule, wherein the plurality of single-stranded oligonucleotides comprises:
(i) a plurality of internal oligonucleotides having overlapping sequence regions with two other oligonucleotides in the plurality, and
(ii) two terminal oligonucleotides designed to be positioned at the 5′ and 3′ terminal ends of the full-length nucleic acid molecule and having an overlapping sequence region with one of the internal oligonucleotides in the plurality,
(b) assembling the plurality of oligonucleotides by primary assembly PCR to obtain assembled double-stranded nucleic acid assembly products,
(c) combining at least a portion of the assembly products obtained in step (b) with a pair of primers, wherein the primers are designed to bind to the 5′ and 3′ terminal ends of the assembly products and performing a PCR amplification reaction to produce amplified assembly products,
wherein step (b) and/or step (c) is conducted in the presence of one or more thermostable mismatch recognition protein.

46. The method of claim 45, further comprising (d) conducting one or more error correction steps, wherein an error correction step comprises:
(iii) denaturing and reannealing the amplified assembly products of step (c) to generate one or more mismatch containing double-stranded nucleic acids, and
(iv) treating the mismatch containing double-stranded nucleic acids with one or more mismatch recognition protein, and
(v) optionally, conducting an amplification reaction.

47. The method of claim 46, wherein the mismatch recognition protein used in step (d) is a mismatch endonuclease or a mismatch binding protein.

48.-49. (canceled)

50. The method of claim 45, wherein the thermostable mismatch recognition protein is as thermostable mismatch endonuclease.

51.-55. (canceled)

56. The method of claim 45, wherein two or more amplified assembly products are pooled prior to conducting the one or more error correction steps.

57. (canceled)
